(19) United States
(12) Patent Application Publication
(10) Pub. No.: US 2007/0259855 A1
(43) Pub. Date: Nov. 8, 2007

(54) THIAZOLYL-DIHYDRO-INDAZOLE

(76) Inventors: Udo Maier, Senden (DE); Matthias Grauert, Biberach (DE); Matthias Hoffmann, Mittelbiberach (DE); Christoph Hoenke, Ingelheim (DE); Anne T. Joergensen, Biberach (DE); Alexander Pautsch, Ulm (DE); Trixi Brandl, Basel (CH); Steffen Breitfelder, Assmannshardt (DE); Stefan Scheuerer, Warthausen (DE); Klaus Erb, Mittelbiberach (DE); Michael Pieper, Biberach (DE); Ingo Pragst, Muenchen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/690,360

(22) Filed: Mar. 23, 2007

(30) Foreign Application Priority Data
Apr. 6, 2006 (EP) 06112299

(51) Int. Cl. A61K 31/429; A61K 31/428; A61K 31/44; A61K 31/517; A61K 31/549; A61K 31/55; A61K 31/551; A61P 11/00; A61P 17/00; A61P 19/00; A61P 29/00; A61P 31/00; A61P 37/00; C07D 417/06; C07D 513/04

(52) U.S. Cl. 514/215; 514/217.05; 514/218; 514/256; 514/266.1; 514/338; 514/366; 514/367; 544/333; 546/270.1; 548/151

(57) ABSTRACT

Disclosed are compounds of general formula (I), 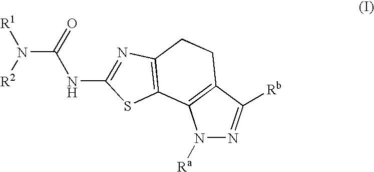
wherein the groups R¹, R², R^{a} and R^{b} have the meanings given in the claims and specification, the tautomers, racemates, enantiomers, diastereomers and the mixtures thereof, and optionally the pharmacologically acceptable acid addition salts, solvates and hydrates thereof, and processes for preparing these thiazolyl-dihydro-indazoles and the use thereof as pharmaceutical compositions.

This application claims priority benefit to EP 06112299, filed Apr. 6, 2006, the entirety of which is incorporated herein.

The present invention relates to new thiazolyl-dihydro-indazoles of general formula (I) 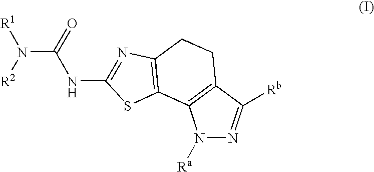
wherein the groups R¹, R², R^{a} and R^{b} have the meanings given in the claims and specification, the tautomers, racemates, enantiomers, diastereomers and the mixtures thereof, and optionally the pharmacologically acceptable acid addition salts, solvates and hydrates thereof, and processes for preparing these thiazolyl-dihydro-indazoles and the use thereof as pharmaceutical compositions.

### BACKGROUND TO THE INVENTION

Phosphatidylinositol-3-kinases (PI3-kinases) are a subfamily of the lipid kinases which catalyse the transfer of a phosphate group to the 3′-position of the inositol ring of phosphoinositides.

They have a role in numerous cell processes such as e.g. cell growth and differentiation processes, the control of cytoskeletal changes and the regulation of intracellular transport processes (Vanhaesebroeck et al., Annu Rev Biochem. 2001; 70:535-602).

PI3-kinases may play a part in numerous tumours, such as e.g. breast cancer, ovarian or pancreatic carcinoma, in tumour types such as carcinomas of the colon, breast or lungs, but particularly in autoimmune diseases such as Crohn's disease or rheumatoid arthritis, for example, or in the cardiovascular system, e.g. in the development of cardiac hypertrophy (Oudit et al., Circulation. Oct. 28, 2003 ;108(17):2147-52). P13-kinase modulators may represent a possible method of anti-inflammatory therapy with comparatively minor side effects (Ward and Finan, Curr Opin Pharmacol. August 2003;3(4):426-34).

PI3-kinase inhibitors for treating inflammatory diseases are known in the literature. Thus, WO 03/072557 discloses 5-phenylthiazole derivatives, WO 04/029055 discloses annelated azolpyrimidines and WO 04/007491 discloses azolidinone-vinyl linked benzene derivatives. Moreover, the two specifications WO 04/052373 and WO 04/056820 disclose benzoxazine and benzoxazin-3-one derivatives.

The aim of the present invention is to provide new compounds which by virtue of their pharmaceutical activity as PI3-kinase modulators may be used therapeutically for the treatment of inflammatory or allergic diseases. Examples of these include inflammatory and allergic respiratory complaints, inflammatory and allergic skin complaints, inflammatory eye diseases, diseases of the nasal mucosa, inflammatory or allergic illnesses which involve autoimmune reactions or kidney inflammation.

### DETAILED DESCRIPTION OF THE INVENTION

Surprisingly it has been found that the above problem is solved by means of compounds of formula (I), wherein the groups R¹, R², R^{a} and R^{b} have the meanings given hereinafter.

It has particularly been found that compounds of formula (I) act as inhibitors of PI3-kinase, particularly as inhibitors of PI3-kinase gamma. Thus the compounds according to the invention may be used for example for the treatment of respiratory complaints.

The present invention therefore relates to compounds of general formula (I), 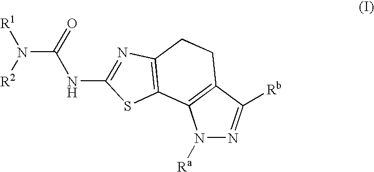
wherein
R^{a} denotes hydrogen or an optionally substituted group selected from among C₁-C₈-alkyl, C₂-C₈-alkenyl, C₂-C₈-alkynyl, C₃-C₈-cycloalkyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl and C₃-C₈-heterocycloalkyl-C₁-C₄-alkyl,
R^{b} denotes hydrogen or an optionally substituted group selected from among C₁-C₈-alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl, CONH₂, C₆-C₁₄-aryl-NH— and C₃-C₈-heterocycloalkyl-NH—;
R¹ denotes hydrogen or an optionally substituted group selected from among C₁-C₈-alkyl, C₃-C₈-cycloalkyl, C₂-C₈-alkenyl, C₂-C₈-alkynyl and C₆-C₁₄-aryl-C₁-C₅-alkyl-;
R² denotes hydrogen or an optionally substituted group selected from among C₁-C₈ alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₆-alkyl, C₉-C₁₃-spiro, C₃-C₈-heterocycloalkyl, C₃-C₈-heterocycloalkyl-C₁-C₆-alkyl- and C₆-C₁₄-aryl-C₁-C₆-alkyl-; or
R¹ and R² together form an optionally substituted five-, six- or seven-membered ring consisting of carbon atoms and optionally 1 to 2 heteroatoms, selected from among oxygen, sulphur and nitrogen or
R¹ and R² together form an optionally substituted nine- to thirteen-membered spirocyclic ring, or
R² denotes a group selected from among general formulae (1l) to (A18) 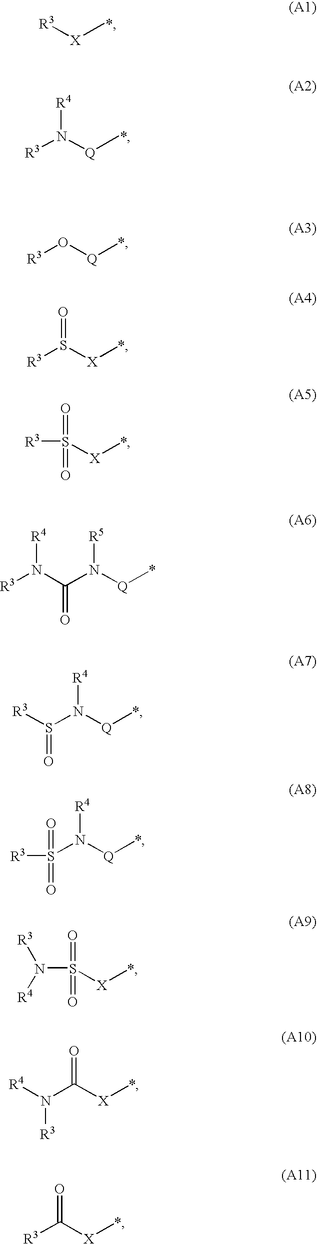 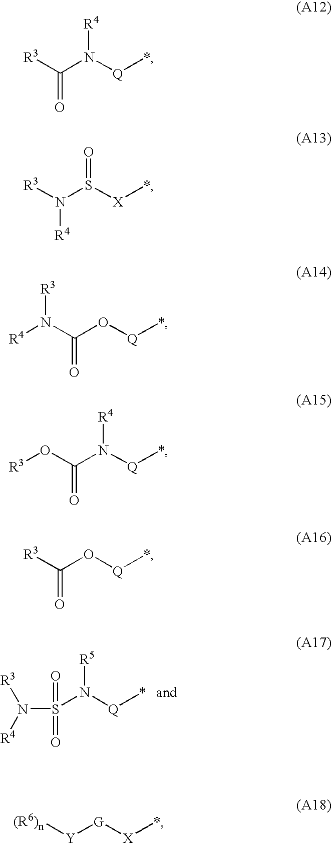
   wherein
X and Y may be linked to the same or different atoms of G, and
X denotes a bond or an optionally substituted group selected from among C₁-C₇-alkylene, C₃-C₇-alkenylene and C₃-C₇-alkynylene, or
X together with R¹, R³ or R⁴ forms a C₁-C₇-alkylene bridge;
Y denotes a bond or optionally substituted C₁-C₄-alkylene;
Q together with R¹, R³ or R⁴ forms a C₁-C₇-alkylene bridge;
Q denotes an optionally substituted group selected from among C₁-C₇-alkylene, C₃-C₇-alkenylene and C₃-C₇-alkynylene;
R³, R⁴, R⁵ which may be identical or different, denote hydrogen or an optionally substituted group selected from among C₁-C₈-alkyl, C₃-C₈-cycloalkyl, C₂-C₆-haloalkyl, C₁-C₄-alkyl-C₃-C₈-cycloalkyl, C₃-C₈-cycloalkyl-C₁-C₄-alkyl, NR⁷R⁸, NR⁷R⁸—C₁-C₄-alkyl, C₁-C₄-alkoxy, C₁-C₄-alkoxy-C₁-C₄-alkyl, C₆-C₁₄-aryl and C₅-C₁₀-heteroaryl, or in each case two of the substituents
R³, R⁴, R⁵ together form an optionally substituted five-, six- or seven-membered ring, consisting of carbon atoms and optionally 1-2 heteroatoms, selected from among oxygen, sulphur and nitrogen;
G denotes a saturated, partially saturated or unsaturated ring system consisting of 3-10 C atoms, wherein optionally up to 6 C atoms are replaced by heteroatoms selected from among nitrogen, oxygen and sulphur;
R⁶ which may be identical or different, denote hydrogen or an optionally substituted group selected from among ═O, C₁-C₈-alkyl, C₃-C₈-cycloalkyl, C₂-C₆-haloalkyl, C₆-C₁₄-aryl, C₅-C₁₀-heteroaryl and C₃-C₈-heterocycloalkyl, or
   a group selected from among NR⁷R⁸, OR⁷, —CO—C₁-C₃-alkyl-NR⁷R⁷R⁸, —O—C₁-C₃-alkyl-NR⁷R⁸, CONR⁷R⁸, NR⁷COR⁸, —CO—C₁-C₃-alkyl-NR⁷(CO)OR⁸, —O(CO)NR⁷R⁸, NR⁷(CO)NR⁸R⁹, NR⁷(CO)OR⁸, (CO)OR⁷, —O(CO)R⁷, COR⁷, (SO)R⁷, (SO₂)R⁷, (SO₂)NR⁷R⁸, NR⁷(SO₂)R⁸, NR⁷(SO₂)NR⁸R⁹, CN and halogen;
n denotes 1, 2 or 3
R⁷, R⁸, R⁹ which may be identical or different, denote hydrogen or an optionally substituted group selected from among C₁-C₈-alkyl, C₃-C₈-cycloalkyl, C₂-C₆-haloalkyl, C₁-C₄-alkyl-C₃-C₈-cycloalkyl, C₃-C₈-cycloalkyl-C₁-C₃-alkyl, C₆-C₁₄-aryl, C₁-C₄-alkyl-C₆-C₁₄-aryl, C₆-C₁₄-aryl-C₁-C₄-alkyl, C₃-C₈-heterocycloalkyl, C₁-C₅-alkyl-C₃-C₈-heterocycloalkyl, C₃-C₈-heterocycloalkyl-C₁-C₄-alkyl, C₁-C₄-alkyl(CO)— and C₁-C₄-alkyl-O(CO)—; or in each case two of the substituents
R⁷, R⁸, R⁹ together form an optionally substituted five-, six- or seven-membered ring, consisting of carbon atoms and optionally 1-2 heteroatoms, selected from among oxygen, sulphur and nitrogen;
   optionally in the form of the tautomers, the racemates, the enantiomers, the diastereomers and the mixtures thereof, as well as optionally the pharmacologically acceptable acid addition salts, solvates and hydrates thereof,
   with the proviso that the following compounds are excluded:
   a) (1-phenyl-4,5-dihydro-1H-pyrazolo[3′,4′:3,4]benzo[1,2-d]thiazol-7-yl)-hydrazinecarboxamide
   b) 1-(2-dimethylamino-ethyl)-3-(1-phenyl-4,5-dihydro-1H-pyrazolo[3′,4′:3,4]benzo[1,2-d]thiazol-7-yl)-urea
   c) 1-(2-morpholin-4-yl-ethyl)-3-(1-phenyl-4,5-dihydro-1H-pyrazolo[3′,4′:3,4]benzo[1,2-d]thiazol-7-yl)-urea
   d) 1-ethyl-3-(1-phenyl-4,5-dihydro-1H-pyrazolo[3′,4′:3,4]benzo[1,2-d]thiazol-7-yl)-urea
   e) 1-methyl-3-(1-phenyl-4,5-dihydro-1H-pyrazolo[3′,4′:3,4]benzo[1,2-d]thiazol-7-yl)-urea
   f) 1,1-dimethyl-3-(1-phenyl-4,5-dihydro-1H-pyrazolo[3′,4′:3,4]benzo[1,2-d]thiazol-7-yl)-urea
   g) morpholine-4-carboxylic acid (1-phenyl-4,5-dihydro-1H-pyrazolo[3′,4′:3,4]benzo[1,2-d]thiazol-7-yl)-amide
   h) [1-(2-chloro-phenyl)-3-isopropyl-4,5-dihydro-1H-pyrazolo[3′,4′:3,4]benzo[1,2-d]thiazol-7-yl]-urea
   i) N-(5.8-dihydro-4H-[1,3]thiazolo[4,5-g]indol-2-yl)-N′-ethylurea
   j) N-ethyl-N′-(8-methyl-5.8-dihydro-4H-[1,3]thiazolo[4,5-g]indol-2-yl)urea
   k) tert-butyl {4-[3-(1-phenyl-4,5-dihydro-1H-pyrazolo[3′,4′:3,4]benzo[1,2-d]thiazol-7-yl)-ureido]-but-2-ynyl}-carbamate
   l) 1-(4-amino-but-2-ynyl)-3-(1-phenyl-4,5-dihydro-1H-pyrazolo[3′,4′:3,4]benzo[1,2-d]thiazol-7-yl)-urea
   m) (1-phenyl-4,5-dihydro-1H-pyrazolo[3′,4′:3,4]benzo[1,2-d]thiazol-7-yl)-urea

Preferred are compounds of formula (I), wherein
X, Y, Q and G may have the meaning specified and
R^{a} denotes hydrogen or a group selected from among C₁-C₈-alkyl, C₂-C₈-alkenyl, C₂-C₈-alkynyl, C₃-C₈-cycloalkyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl and C₃-C₈-heterocycloalkyl-C₁-C₄-alkyl, which may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, C₁-C₄-alkoxy, CN, NO₂, NR¹⁰R¹¹, OR¹⁰, COR¹⁰, COOR¹⁰, CONR¹⁰R¹¹, NR¹⁰COR¹¹, NR¹⁰(CO)NR¹¹R¹², O(CO)NR¹⁰R¹¹, NR¹⁰(CO)OR¹¹, SO₂R¹⁰, SOR¹⁰, SO₂NR¹⁰R¹¹, NR¹⁰SO₂NR¹¹R¹² and NR¹⁰SO₂R¹¹;
R¹⁰, R¹¹, R¹² which may be identical or different, denote hydrogen or a group selected from among C₁-C₆-alkyl, C₂-C₆-alkenyl, C₂-C₆-alkynyl, C₃-C₈-cycloalkyl and C₁-C₆ haloalkyl; or
   in each case two of the groups
R¹⁰, R¹¹, R¹² together form a five-, six- or seven-membered ring, consisting of carbon atoms and optionally 1-2 heteroatoms, selected from among oxygen, sulphur and nitrogen;
R^{b} denotes hydrogen or a group selected from among C₁-C₈-alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl, CONH₂, C₆-C₁₄-aryl-NH, C₃-C₈-heterocycloalkyl-NH, which may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, OMe, CN, NH₂, NHMe and NMe₂;
R¹ denotes hydrogen or a group selected from among C₁-C₈-alkyl, C₃-C₈-cycloalkyl, C₂-C₈-alkenyl, C₂-C₈-alkynyl and C₆-C₁₄-aryl-C₁-C₅-alkyl, which may optionally be substituted by one or more of the groups, which may be identical or different, selected from among halogen, NH₂, OH, CN, C₁-C₆-alkyl, OMe, —NH(CO)-alkyl and —(CO)O-alkyl,
R² denotes hydrogen or a group selected from among C₁-C₈ alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₆-alkyl, C₉-C₁₃-spiro, C₃-C₈-heterocycloalkyl, C₃-C₈-heterocycloalkyl-C₁-C₆-alkyl- and C₆-C₁₄-aryl-C₁-C₆-alkyl, which may optionally be substituted by one or more of the groups, which may be identical or different, selected from among halogen, NH₂, OH, CN, C₁-C₆-alkyl, OMe, —NH(CO)-alkyl and —(CO)O-alkyl. or
R¹ and R² together form a five-, six- or seven-membered ring consisting of carbon atoms and optionally 1 to 2 heteroatoms, selected from among oxygen, sulphur and nitrogen, which may optionally be substituted by one or more of the groups, which may be identical or different, selected from among heterocycloalkyl, halogen, NH₂, OH, CN, C₁-C₆-alkyl, OMe, —NH(CO)-alkyl and —(CO)O-alkyl. or
R¹ and R² together form an optionally substituted nine- to thirteen-membered spirocyclic ring, or
R² denotes a group selected from among general formulae (A1) to (A18) 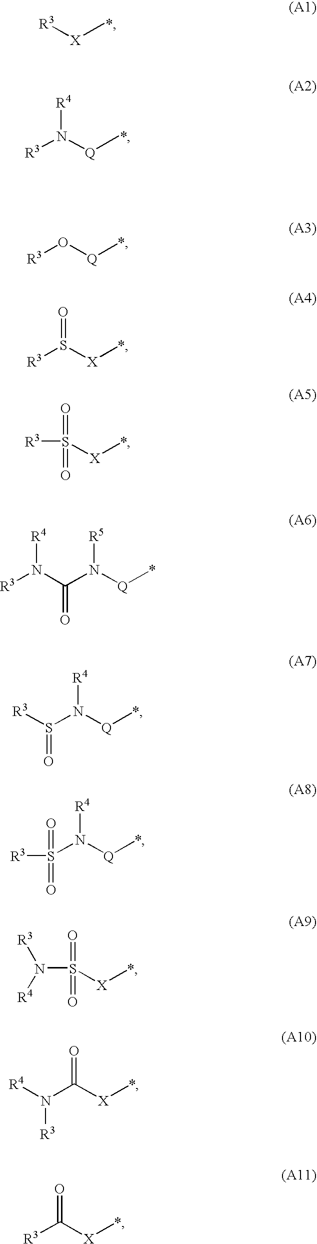 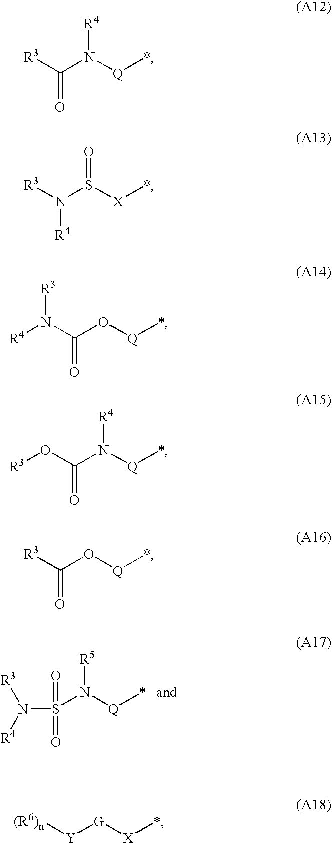
   wherein
R³, R⁴, R⁵ which may be identical or different, denote hydrogen or a group selected from among C₁-C₈-alkyl, C₃-C₈-cycloalkyl, C₂-C₆-haloalkyl, C₁-C₄-alkyl-C₃-C₈-cycloalkyl, C₃-C₈-cycloalkyl-C₁-C₄-alkyl, NR⁷R⁸, NR⁷R⁸-C₁-C₄-alkyl, C₁-C₄-alkoxy, C₁-C₄-alkoxy-C₁-C₄-alkyl, C₆-C₁₄-aryl and C₅-C₁₀-heteroaryl, which may optionally be substituted by one or more of the groups, which may be identical or different, selected from among halogen, NH₂, OH, CN, NR⁹R¹⁰, —NH(CO)—C₁-C₄-alkyl and MeO, or in each case two of the substituents
R³, R⁴, R⁵ together form a five-, six- or seven-membered ring, consisting of carbon atoms and optionally 1-2 heteroatoms, selected from among oxygen, sulphur and nitrogen; which may optionally be substituted by one or more of the groups, which may be identical or different, selected from among halogen, NH₂, OH, CN, NR⁹R¹⁰, —NH(CO)—C₁-C₄-alkyl and MeO,
R⁶ which may be identical or different, denote hydrogen or a group, selected from among, C₁-C₈-alkyl, C₃-C₈-cycloalkyl, C₂-C₆-haloalkyl, C₆-C₁₄-aryl, C₅-C₁₀-heteroaryl and C₃-C₈-heterocycloalkyl, which may optionally be substituted by one or more of the groups, which may be identical or different, selected from among, NH₂, NHMe, NMe₂, OH, OMe, CN and C₁-C₆-alkyl, —(CO)O—C₁-C₆-alkyl or
   a group selected from among ═O, NR⁷R⁸, OR⁷, —CO—C₁-C₃-alkyl-NR⁷R⁸, —O—C₁-C₃-alkyl-NR⁷R⁸, CONR⁷R⁸, NR⁷COR⁸, —CO—C₁-C₃-alkyl-NR⁷(CO)OR⁸, —O(CO)NR⁷R⁸, NR⁷(CO)NR⁸R⁹, NR⁷(CO)OR⁸, (CO)OR⁷, —O(CO)R⁷, COR⁷, (SO)R⁷, (SO₂)R⁷, (SO₂)NR⁷R⁸, NR⁷(SO₂)R⁸, NR⁷(SO₂)NR⁸R⁹, CN and halogen;
n denotes 1, 2 or 3
R⁷, R⁸, R⁹ which may be identical or different, denote hydrogen or a group selected from among C₁-C₈-alkyl, C₃-C₈-cycloalkyl, C₂-C₆-haloalkyl, C₁-C₄-alkyl-C₃-C₈-cycloalkyl, C₃-C₈-cycloalkyl-C₁-C₃-alkyl, C₆-C₁₄-aryl, C₁-C₄-alkyl-C₆-C₁₄-aryl, C₆-C₁₄-aryl-C₁-C₄-alkyl, C₃-C₈-heterocycloalkyl, C₁-C₅-alkyl-C₃-C₈-heterocycloalkyl, C₃-C₈-heterocycloalkyl-C₁-C₄-alkyl, C₁-C₄-alkyl(CO)— and C₁-C₄-alkyl-O(CO), which may optionally be substituted by one or more of the groups, which may be identical or different, selected from among halogen, NH₂, OH, CN, OMe, NHMe, NMe₂, C₁-C₆-alkyl and (CO)O C₁-C₆-alkyl, or in each case two of the substituents
R⁷, R⁸, R⁹ together form a five-, six- or seven-membered ring, consisting of carbon atoms and optionally 1-2 heteroatoms, selected from among oxygen, sulphur and nitrogen; which may optionally be substituted by one or more of the groups, which may be identical or different, selected from among halogen, NH₂, OH, CN, OMe, NHMe, NMe₂, C₁-C₆-alkyl and (CO)O C₁-C₆-alkyl.

Also preferred are compounds of formula (I), wherein
R^{a} and R¹ to R¹² may have the meaning specified and
R^{b} denotes a group selected from among C₁-C₈-alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl, CONH₂, C₆-C₁₄-aryl-NH, C₃-C₈-heterocycloalkyl-NH, which may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, OMe, CN, NH₂, NHMe and NMe₂.

Also preferred are compounds of formula (I), wherein
R¹ to R¹² may have the meaning specified and
R^{a} denotes C₆-C₁₄-aryl or a saturated ring system consisting of 5-6 C atoms, wherein optionally up to 4 C atoms are replaced by nitrogen atoms, wherein R^{a} may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, C₁-C₄-alkoxy, CN, NO₂, NR¹⁰R¹¹, OR¹⁰, COR¹⁰, COOR¹⁰, CONR¹⁰R¹¹, NR¹⁰COR¹¹, NR¹⁰(CO)NR¹¹R¹², O(CO)NR¹⁰R¹¹, NR¹⁰(CO)OR¹¹, SO₂R¹⁰, SOR¹⁰, SO₂NR¹⁰R¹¹, NR¹⁰SO₂NR¹¹R¹² and NR¹⁰SO₂R¹¹;
R^{b} denotes hydrogen or a group selected from among C₃-C₈-cycloalkyl, C₆-C₁₄-aryl, C₅-C₁₀-heteroaryl, C₆-C₁₄-aryl-NH, which may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, OMe, CN, NH₂, NHMe and NMe₂.

Also preferred are compounds of formula (I), wherein
R^{a} and R^{b} may have the meaning specified and
R¹ denotes hydrogen, C₁-C₅-alkyl or C₃-C₈-cycloalkyl,
R² denotes hydrogen, C₁-C₅-alkyl or C₃-C₈-cycloalkyl, or
R¹ and R² together form an optionally substituted five- or six-membered ring consisting of carbon atoms and optionally 1 to 2 nitrogen atoms, or
R¹ and R² together form an optionally substituted nine- to thirteen-membered spirocyclic ring, or
R¹, R² which may be identical or different, denote a group selected from among general formulae (A2), (A3), (A8), (A10), (A11) and (A12), wherein
X denotes a bond or an optionally substituted C₁-C₃-alkylene, or
X together with R¹, R³ or R⁴ forms a 5- or 6-membered heterocyclic group,
Q denotes an optionally substituted C₁-C₃-alkylene, or
Q together with R¹, R³ or R⁴ forms a C₁-C₇-alkylene bridge
R³, R⁴, R⁵ which may be identical or different, denote hydrogen or an optionally substituted group selected from among C₁-C₄-alkyl, C₁-C₄-alkoxy, C₃-C₆-cycloalkyl and C₅-C₁₀-heteroaryl, or in each case two of the substituents
R³, R⁴, R⁵ together form an optionally substituted five- or six-membered ring, consisting of carbon atoms and optionally 1-2 heteroatoms, selected from among oxygen and nitrogen.

Particularly preferred are compounds of formula (I), wherein
R^{a} and R^{b} may have the meaning specified and
R¹ denotes H or Me,
R² denotes hydrogen or a group of general formula (A18), wherein
X denotes a bond or an optionally substituted group selected from among C₁-C₇-alkylene, C₃-C₇-alkenylene and C₃-C₇-alkynylene, or
X together with R¹ forms a C₁-C₇-alkylene bridge
Y denotes a bond or methylene, ethylene;
X and Y may be linked to the same or different atoms of G, and
G denotes a saturated, partially saturated or unsaturated ring system consisting of 3-10 C atoms, wherein optionally up to 6 C atoms are replaced by heteroatoms selected from among nitrogen, oxygen and sulphur;
R⁶ which may be identical or different, denote hydrogen or an optionally substituted group selected from among ═O, C₁-C₄-alkyl, C₃-C₆-cycloalkyl, C₆-C₁₄-aryl, C₅-C₆-heterocycloalkyl and C₅-C₆-heteroaryl, or a group selected from among OR⁷, NR⁷R⁸, —O—C₁-C₃-alkyl-NR⁷R⁸, CONR⁷R⁸, CO—C₁-C₃-alkyl-NR⁷R⁸, NR⁷COR⁸, NR⁷(CO)OR⁸, —CO—C₁-C₃-alkyl-NR⁷(CO)OR⁸, NR⁷(CO)NR⁸R⁹, NR⁷(CO)OR⁸, (CO)OR⁷, COR⁷, (SO₂)R⁷ and CN,
n denotes 1 or 2
R⁷, R⁸, R⁹ which may be identical or different, denote hydrogen or an optionally substituted group selected from among C₁-C₅-alkyl, C₁-C₄-alkyl-C₆-C₁₄-aryl, C₃-C₆-heterocycloalkyl and C₁-C₅-alkyl-C₃-C₈-heterocyclo-alkyl, or in each case two of the substituents
R⁷, R⁸, R⁹ together form an optionally substituted five- or six-membered ring, consisting of carbon atoms and optionally 1-2 heteroatoms, selected from among oxygen and nitrogen.

In another aspect the invention relates to compounds of formula (I) for use as pharmaceutical compositions.

The invention further relates to the use of the compounds of formula (I) for preparing a pharmaceutical composition for the treatment of diseases in whose pathology an activity of PI3-kinases is implicated, wherein therapeutically effective doses of the compounds of formula (I) may confer a therapeutic benefit.

The invention further relates to the use of the compounds of formula (I), for preparing a pharmaceutical composition for the treatment of inflammatory and allergic diseases of the airways.

The invention further relates to the use of the compounds of formula (I), for preparing a pharmaceutical composition for the treatment of a disease, which is selected from among chronic bronchitis, bronchitis caused by bacterial or viral infections or fungi or helminths, allergic bronchitis, toxic bronchitis, chronic obstructive bronchitis (COPD), asthma (intrinsic or allergic), paediatric asthma, bronchiectases, allergic alveolitis, allergic or non-allergic rhinitis, chronic sinusitis, cystic fibrosis or mucoviscidosis, alpha1-antitrypsin deficiency, coughing, pulmonary emphysema, interstitial lung diseases, alveolitis, hyperreactive airways, nasal polyps, pulmonary oedema, pneumonitis of various causes, such as radiation-induced or caused by aspiration or infection, collagenoses such as lupus erythematodes, systemic scleroderma, sarcoidosis and Boeck's disease.

The invention further relates to the use of the compounds of formula (I), for preparing a pharmaceutical composition for the treatment of inflammatory and allergic diseases of the skin.

The invention further relates to the use of the compounds of formula (I), for preparing a pharmaceutical composition for the treatment of a disease which is selected from among psoriasis, contact dermatitis, atopical dermatitis, alopecia areata (circular hair loss), erythema exsudativum multiforme (Stevens-Johnson Syndrome), dermatitis herpetiformis, sclerodermy, vitiligo, nettle rash (urticaria), lupus erythematodes, follicular and surface pyoderma, endogenous and exogenous acne, acne rosacea and other inflammatory and allergic or proliferative skin complaints.

The invention further relates to the use of the compounds of formula (I), for preparing a pharmaceutical composition for the treatment of inflammation of the eye.

The invention further relates to the use of the compounds of formula (I), for preparing a pharmaceutical composition for the treatment a disease which is selected from among conjunctivitis of various kinds, such as e.g. caused by fungal or bacterial infections, allergic conjunctivitis, irritable conjunctivitis, conjunctivitis caused by drugs, keratitis and uveitis.

The invention further relates to the use of the compounds of formula (I), for preparing a pharmaceutical composition for the treatment of diseases of the nasal mucosa.

The invention further relates to the use of the compounds of formula (I), for preparing a pharmaceutical composition for the treatment of a disease, which is selected from among allergic rhinitis, allergic sinusitis and nasal polyps.

The invention further relates to the use of the compounds of formula (I), for preparing a pharmaceutical composition for the treatment of inflammatory or allergic conditions involving autoimmune reactions.

The invention further relates to the use of the compounds of formula (I), for preparing a pharmaceutical composition for the treatment of a disease which is selected from among Crohn's disease, ulcerative colitis, systemic lupus erythematodes, chronic hepatitis, multiple sclerosis, rheumatoid arthritis, psoriatric arthritis, osteoarthritis, rheumatoid spondylitis.

The invention further relates to the use of the compounds of formula (I), for preparing a pharmaceutical composition for the treatment of kidney inflammation. The invention further relates to the use of the compounds of formula (I), for preparing a pharmaceutical composition for the treatment of a disease which is selected from among glomerulonephritis, interstitial nephritis and idiopathic nephrotic syndrome.

Of particular importance according to the invention is a pharmaceutical formulation containing a compound of formula (I).

Preferred is an inhaled pharmaceutical formulation containing a compound of formula (I).

Also preferred is an orally administered pharmaceutical formulation containing a compound of formula (I).

Terms and Definitions Used

By alkyl groups as well as alkyl groups which are part of other groups are meant branched and unbranched alkyl groups with 1 to 10 carbon atoms, preferably 1-6, particularly preferably 1-4 carbon atoms, are meant for example: methyl, ethyl, propyl, butyl, pentyl, hexyl, heptyl, octyl, nonyl and decyl. Unless stated otherwise, the above terms propyl, butyl, pentyl, hexyl, heptyl, octyl, nonyl and decyl include all the possible isomeric forms. For example the term propyl includes the two isomeric groups n-propyl and iso-propyl, the term butyl includes n-butyl, iso-butyl, sec. butyl and tert.-butyl, the term pentyl includes isopentyl, neopentyl etc. In the above-mentioned alkyl groups, unless otherwise specified, one or more hydrogen atoms may be replaced by other groups. For example these alkyl groups may be substituted by the halogen atoms fluorine, chlorine, bromine or iodine. The substituents fluorine or chlorine are preferred. It is also possible for all the hydrogen atoms of the alkyl group to be replaced.

By alkyl bridge is meant, unless stated otherwise, branched and unbranched double-bonded alkyl groups with 4 to 7 carbon atoms, for example, n-butylene, iso-butylene, sec. butylene and tert.-butylene, pentylene, iso-pentylene, neopentylene, etc. bridges. Particularly preferred are n-butylene or n-pentylene bridges. In the above-mentioned alkyl bridges 1 to 2 C atoms may optionally be replaced by one or more heteroatoms selected from among oxygen or sulphur.

By the term “C_{1 6}-alkylene” (including those which are part of other groups) are meant branched and unbranched alkylene groups with 1 to 6 carbon atoms and by the term “C_{1 4}-alkylene” are meant branched and unbranched alkylene groups with 1 to 4 carbon atoms. Preferred are alkylene groups with 1 to 4 carbon atoms. Examples include: methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene or hexylene. Unless stated otherwise, the definitions propylene, butylene, pentylene and hexylene include all the possible isomeric forms of the groups in question with the same number of carbons. Thus, for example, propyl also includes 1-methylethylene and butylene includes 1-methylpropylene, 1,1-dimethylethylene, 1,2-dimethylethylene.

Examples of alkenyl groups (including those which are part of other groups) are branched and unbranched alkenyl groups with 2 to 10 carbon atoms, preferably 2-6 carbon atoms, particularly preferably 2-3 carbon atoms, provided that they have at least one double bond. Examples include: ethenyl, propenyl, butenyl, pentenyl etc. Unless stated otherwise, the above-mentioned terms propenyl, butenyl etc. include all the possible isomeric forms. For example the term butylene includes n-butenyl, 1-methylpropenyl, 2-methylpropenyl, 1,1-dimethylethenyl, 1,2-dimethylethenyl etc.

In the above-mentioned alkenyl groups, unless otherwise stated, optionally one or more hydrogen atoms may optionally be replaced by other groups. For example these alkyl groups may be substituted by the halogen atoms fluorine, chlorine, bromine or iodine. The substituents fluorine and chlorine are preferred. Particularly preferred is the substituent chlorine. Optionally all the hydrogen atoms of the alkenyl group may be replaced.

By the term “C₂₋₆-alkenylene” (including those which are part of other groups) are meant branched and unbranched alkenylene groups with 2 to 6 carbon atoms and by the term “C₂₋₄-alkenylene” are meant branched and unbranched alkylene groups with 2 to 4 carbon atoms. Alkenylene groups with 2 to 4 carbon atoms are preferred. Examples include: ethenylene, propenylene, 1-methylethenylene, butenylene, 1-methylpropenylene, 1,1-dimethylethenylene, 1,2-dimethylethenylene, pentenylene, 1,1-dimethylpropenylene, 2,2-dimethylpropenylene, 1,2-dimethylpropenylene, 1,3-dimethylpropenylene or hexenylene. Unless stated otherwise, the definitions propenylene, butenylene, pentenylene and hexenylene include all the possible isomeric forms of the groups in question with the same number of carbons. Thus, for example, propenyl also includes 1-methylethenylene and butenylene includes 1-methylpropenylene, 1,1-dimethylethenylene, 1,2-dimethylethenylene.

Examples of alkynyl groups (including those which are part of other groups) are branched and unbranched alkynyl groups with 2 to 10 carbon atoms, provided that they have at least one triple bond, for example ethynyl, propargyl, butynyl, pentynyl, hexynyl etc., preferably ethynyl or propynyl.

Preferred are alkynyl groups with 2 to 4 carbon atoms. Examples include: ethynyl, propynyl, butynyl, pentynyl, or hexynyl. Unless stated otherwise, the definitions propynyl, butynyl, pentynyl and hexynyl include all the possible isomeric forms of the groups in question. Thus, for example propynyl includes 1-propynyl and 2-propynyl, butynyl includes 1-, 2- and 3-butynyl, 1-methyl-1-propynyl, 1-methyl-2-propynyl etc.

In the above-mentioned alkynyl groups one or more hydrogen atoms may optionally be substituted by other groups unless stated otherwise. For example these alkyl groups may be substituted by the halogen atoms fluorine, chlorine, bromine or iodine. The substituents fluorine and chlorine are preferred. Optionally all the hydrogen atoms of the alkynyl group may be replaced.

By the term “C₂₋₆-alkynylene” (including those which are part of other groups) are meant branched and unbranched alkynylene groups with 2 to 6 carbon atoms and by the term “C₂₋₄-alkynylene” are meant branched and unbranched alkylene groups with 2 to 4 carbon atoms. Preferred are alkynylene groups with 2 to 4 carbon atoms. Examples include: ethynylene, propynylene, 1-methylethynylene, butynylene, 1-methylpropynylene, 1,1-dimethylethynylene, 1,2-dimethylethynylene, pentynylene, 1,1-dimethylpropynylene, 2,2-dimethylpropynylene, 1,2-dimethylpropynylene, 1,3-dimethylpropynylene or hexynylene. Unless stated otherwise, the definitions propynylene, butynylene, pentynylene and hexynylene include all the possible isomeric forms of the groups in question with the same number of carbons. Thus, for example propynyl also includes 1-methylethynylene and butynylene includes 1-methylpropynylene, 1,1-dimethylethynylene, 1,2-dimethylethynylene.

By cycloalkyl groups (including those which are part of other groups) are meant saturated cycloalkyl groups with 3-8 carbon atoms, for example cyclopropyl, cyclobutyl, cyclopentyl, cyclohexyl, cycloheptyl or cyclooctyl, preferably cyclopropyl, cyclopentyl or cyclohexyl, while each of the above-mentioned cycloalkyl groups may optionally carry one or more substituents or be anellated to a benzene ring. Moreover the cycloalkyl groups may form, in addition to monocyclic groups, bicyclic, bridged or spirocyclic ring systems.

By cycloalkenyl (including those which are part of other groups) are meant cyclic alkyl groups with 5 to 8, preferably 5 or 6 carbon atoms, which contain one or two double bonds. Examples include: cyclopentenyl, cyclopentadienyl, cyclohexenyl, cyclohexadienyl, cycloheptenyl, cycloheptadienyl, cyclooctenyl or cyclooctadienyl. Moreover the cycloalkenyl groups may form, in addition to monocyclic groups, bicyclic, bridged or spirocyclic ring systems.

By cycloalkynyl (including those which are part of other groups) are meant cyclic alkyl groups with 5 to 8, preferably 5 or 6 carbon atoms, which contain one or two triple bonds. Examples of these include: cyclopentynyl, cyclopentadiynyl, cyclohexynyl, cyclohexadiynyl, cycloheptynyl, cycloheptadiynyl, cyclooctynyl or cyclooctadiynyl. Moreover the cycloalkynyl groups may form, in addition to monocyclic ring systems, bicyclic, bridged or spirocyclic ring systems.

By haloalkyl (including those which are part of other groups) are meant branched and unbranched alkyl groups with 1 to 6 carbon atoms, wherein one or more hydrogen atoms are replaced by a halogen atom selected from among fluorine, chlorine or bromine, preferably fluorine and chlorine. By the term “C₁₋₄-haloalkyl” are meant correspondingly branched and unbranched alkyl groups with 1 to 4 carbon atoms, wherein one or more hydrogen atoms are replaced as described above. C_{1 4}-haloalkyl is preferred. Examples of these include: CH₂F, CHF₂, CF₃.

The term aryl denotes an aromatic ring system with 6 to 14 carbon atoms, preferably 6 or 10 carbon atoms, for example phenyl or naphthyl, preferably phenyl, which, unless otherwise described, may have one or more substituents, for example. Moreover each of the above-mentioned aryl systems may optionally be anellated to a heterocycloalkyl group or a cycloalkyl group. Examples include: 2,3-dihydro-benzo[1,4]dioxine, benzo[1,3]dioxole, 1,2,3,4-tetrahydro-naphthalene and 3,4-dihydro-1H-quinolin-2-one.

By heterocycloalkyl groups are meant, unless otherwise described in the definitions, 5-, 6- or 7-membered, saturated or unsaturated, bridged, mono- or bicyclic heterocycles wherein up to four C atoms may be replaced by one or more heteroatoms selected from among oxygen, nitrogen or sulphur, for example tetrahydrofuran, tetrahydrofuranone, γ-butyrolactone, α-pyran, ≡-pyran, dioxolane, tetrahydropyran, dioxane, dihydrothiophene, thiolane, dithiolane, pyrroline, pyrrolidine, pyrazoline, pyrazolidine, imidazoline, imidazolidine, tetrazole, piperidine, pyridazine, pyrimidine, pyrazine, piperazine, triazine, tetrazine, morpholine, thiomorpholine, diazepan, oxazine, tetrahydro-oxazinyl, isothiazole, pyrazolidine, preferably pyrazolyl, pyrrolidinyl, piperidinyl, piperazinyl or tetrahydro-oxazinyl, while the heterocycle may optionally be substituted, preferably by fluorine or methyl. The ring may be linked to the molecule through a carbon atom or if available through a nitrogen atom.

Unless otherwise mentioned, a heterocyclic ring may be provided with a keto group. Examples of these include.

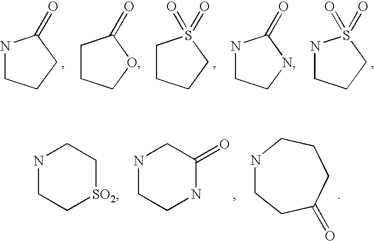

Examples of 5-10-membered bicyclic heterorings include pyrrolizine, indole, indolizine, isoindole, indazole, purine, quinoline, isoquinoline, benzimidazole, benzofuran, benzopyran, benzothiazole, benzothiazole, benzisothiazole, pyridopyrimidine, pteridine, pyrimidopyrimidine,

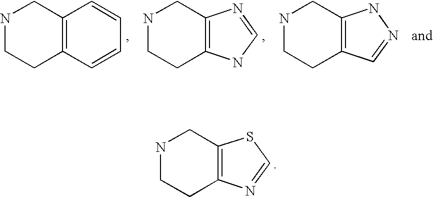

Examples of heteroaryl include 5-10-membered mono- or bicyclic heteroaryl rings in which up to three C atoms may be replaced by one or more heteroatoms selected from among oxygen, nitrogen or sulphur, while these may contain so many conjugated double bonds that an aromatic system is formed. Each of the above-mentioned heterocycles may optionally also be anellated to a benzene ring.

Preferred examples of anellated heteraryl groups are: benzimidazole, indole and pyrimidopyrimidine. Moreover each of the above-mentioned heterocycles may optionally be anellated to a heterocycloalkyl group or a cycloalkyl group.

The heteroaryl rings may, for example, unless otherwise described, carry one or more substituents, preferably halogen or methyl.

The ring may be linked to the molecule through a carbon atom or if present through a nitrogen atom. The following are examples of five- or six-membered heterocyclic aromatic groups:

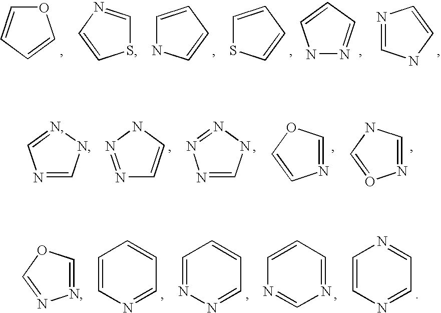

Examples of 5-10-membered bicyclic heteroaryl rings include pyrrolizine, indole, indolizine, isoindole, indazole, purine, quinoline, isoquinoline, benzimidazole, benzofuran, benzopyran, benzothiazole, benzothiazole, benzoisothiazole, pyridopyrimidine, pteridine, pyrimidopyrimidine.

By the term heterocyclic spiro rings (“spiro”) are meant 5-10 membered, spirocyclic rings which may optionally contain one, two or three heteroatoms, selected from among oxygen, sulphur and nitrogen, while the ring may be connected to the molecule via a carbon atom or, if present, via a nitrogen atom. Unless otherwise stated, a spirocyclic ring may be provided with a keto group. Examples include:

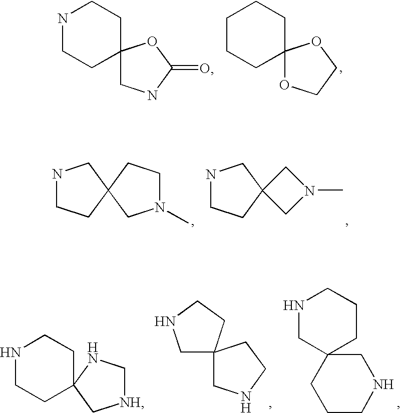

By the term “optionally substituted” is meant, unless stated otherwise, within the scope of the invention the above-mentioned group, optionally substituted by a lower-molecular group. Examples of lower-molecular groups regarded as chemically meaningful are groups consisting of 1-200 atoms. Preferably such groups have no negative effect on the pharmacological efficacy of the compounds. For example the groups may comprise:
Straight-chain or branched carbon chains, optionally interrupted by heteroatoms, optionally substituted by rings, heteroatoms or other common functional groups.
Aromatic or non-aromatic ring systems consisting of carbon atoms and optionally heteroatoms, which may in turn be substituted by functional groups.
A number of aromatic or non-aromatic ring systems consisting of carbon atoms and optionally heteroatoms which may be linked by one or more carbon chains, optionally interrupted by heteroatoms, optionally substituted by heteroatoms or other common functional groups.

“═O” denotes an oxygen atom linked by a double bond.

The term halogen generally denotes fluorine, chlorine, bromine or iodine.

The compounds according to the invention may occur in the form of the individual optical isomers, mixtures of the individual enantiomers, diastereomers or racemates, in the form of the tautomers as well as in the form of the free bases or the corresponding acid addition salts with pharmacologically acceptable acids—such as for example acid addition salts with hydrohalic acids, for example hydrochloric or hydrobromic acid, or organic acids, such as for example oxalic, fumaric, diglycolic or methanesulphonic acid.

Where a hyphen open on one side “-” is used in the structural formula of a substituent, this hyphen is to be understood as the linkage point to the remainder of the molecule. The substituent replaces the corresponding groups R², R⁶, etc. If no hyphen open on one side is used in the structural formula of a substituent, the linkage point to the remainder of the molecule is clear from the structural formula itself.

The substituent R^{a} may be hydrogen or an optionally substituted group selected from among C₁-C₈-alkyl, C₂-C₈-alkenyl, C₂-C₈-alkynyl, C₃-C₈-cycloalkyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl and C₃-C₈-heterocycloalkyl-C₁-C₄-alkyl,

wherein R^{a} may preferably be unsubstituted or substituted by a group selected from among C₁-C₆-alkyl, C₂-C₆-alkenyl, C₂-C₆-alkynyl, C₃-C₈-cycloalkyl, C₁-C₆-haloalkyl, halogen, OH, C₁-C₄-alkoxy, CN, NO₂, NR¹⁰R¹¹, OR¹⁰, COR¹⁰, COOR¹⁰, CONR¹⁰R¹¹, NR¹⁰CR¹¹, NR¹⁰(CO)NR¹¹R¹², O(CO)NR¹⁰R¹¹, NR¹⁰(CO)OR¹¹, SO₂R¹⁰, SOR¹⁰, SO₂NR¹⁰R¹¹, NR¹⁰SO₂NR¹¹R¹² and NR¹⁰SO₂R¹¹, particularly preferably SO2NH2, Me, Et, cyclopentyl, Cl and F. Preferably R^{a} denotes C₆-C₁₄-aryl or a saturated ring system consisting of 5-6 C atoms, wherein optionally up to 4 C atoms are replaced by nitrogen atoms. Particularly preferably R^{a} is phenyl or piperidine.

The substituents R¹⁰, R¹¹, R¹² which may be identical or different, may represent hydrogen or a group selected from among C₁-C₆-alkyl, C₂-C₆-alkenyl, C₂-C₆-alkynyl, C₃-C₈-cycloalkyl and C₁-C₆ haloalkyl; or

in each case two of the groups

R¹⁰, R¹¹, R¹² together form a five-, six- or seven-membered ring, consisting of carbon atoms and optionally 1-2 heteroatoms, selected from among oxygen, sulphur and nitrogen.

The substituent R^{b} may be hydrogen or an optionally substituted group selected from among C₁-C₈-alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl, CONH₂, C₆-C₁₄-aryl-NH, C₃-C₈-heterocycloalkyl-NH, represent, which may preferably be unsubstituted or substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, OMe, CN, NH₂, NHMe and NMe₂.

Preferably R^{b} denotes hydrogen or a group selected from among C₃-C₈-cycloalkyl, C₆-C₁₄-aryl, C₅-C₁₀-heteroaryl, C₆-C₁₄-aryl-NH, which may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, OMe, CN, NH₂, NHMe, NMe₂.

Particularly preferably R^{b} denotes an unsubstituted group selected from among hydrogen, pyrimidine, pyridine, phenyl and cyclopropyl.

The substituent R¹ may represent hydrogen or an optionally substituted group selected from among C₁-C₈-alkyl, C₃-C₈-cycloalkyl, C₂-C₈-alkenyl, C₂-C₈-alkynyl and C₆-C₁₄-aryl-C₁-C₅-alkyl-. Preferably R¹ denotes hydrogen, C₁-C₅-alkyl or C₃-C₈-cycloalkyl. Particularly preferably the substituent R¹ denotes hydrogen or a group selected from among methyl, ethyl, propyl, cyclopropyl and piperidine, particularly preferably R¹ denotes hydrogen or methyl.

The substituent R¹ may preferably be substituted by one or more of the groups, which may be identical or different, selected from among halogen, NH₂, OH, CN, C₁-C₆-alkyl, OMe, —NH(CO)alkyl and —(CO)O—C₁-C₄-alkyl.

The substituent R² may represent hydrogen or an optionally substituted group selected from among C₁-C₈ alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₆-alkyl, C₉-C₁₃-spiro, C₃-C₈-heterocycloalkyl, C₃-C₈-heterocycloalkyl-C₁-C₆-alkyl- and C₆-C₁₄-aryl-C₁-C₆-alkyl-. Preferably R² denotes hydrogen, C₁-C₅-alkyl or C₃-C₈-cycloalkyl. Particularly preferably R² denotes hydrogen or a group selected from among methyl, ethyl, propyl, cyclopropyl and piperidine.

The substituent R² may preferably be substituted by one or more of the groups, which may be identical or different, selected from among halogen, NH₂, OH, CN, C₁-C₆-alkyl, OMe, —NH(CO)alkyl and —(CO)O—C₁-C₄-alkyl.

The substituents R¹ and R² may together form an optionally substituted, five-, six- or seven-membered ring consisting of carbon atoms and optionally 1 to 2 heteroatoms, selected from among oxygen, sulphur and nitrogen, preferably nitrogen. Particularly preferably the group NR¹R² denotes an optionally substituted pyrrolidinyl group.

The ring formed from the substituents R¹ and R² may preferably be substituted by one or more of the groups, which may be identical or different, selected from among heterocycloalkyl, halogen, NH₂, OH, CN, C₁-C₆-alkyl, OMe, —NH(CO)alkyl and —(CO)O—C₁-C₄-alkyl.

The substituents R¹ and R² may together form an optionally substituted nine- to thirteen-membered spirocyclic ring.

The substituent R² may furthermore denote a group selected from among general formulae (A1) to (A18)

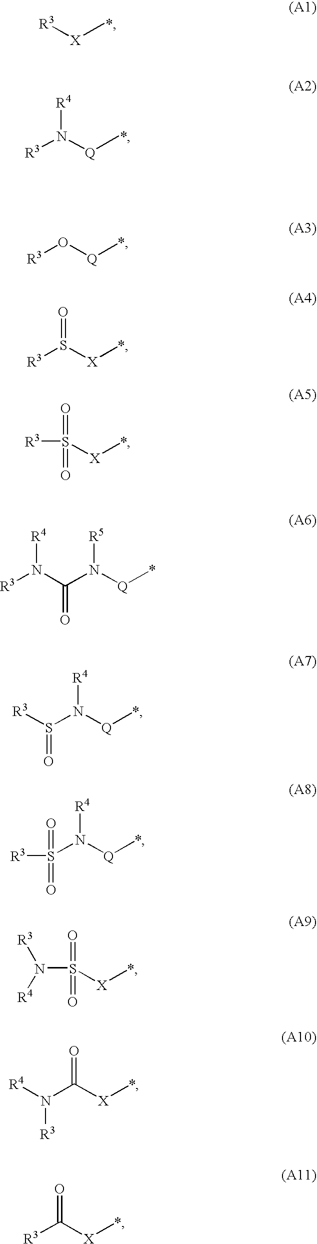

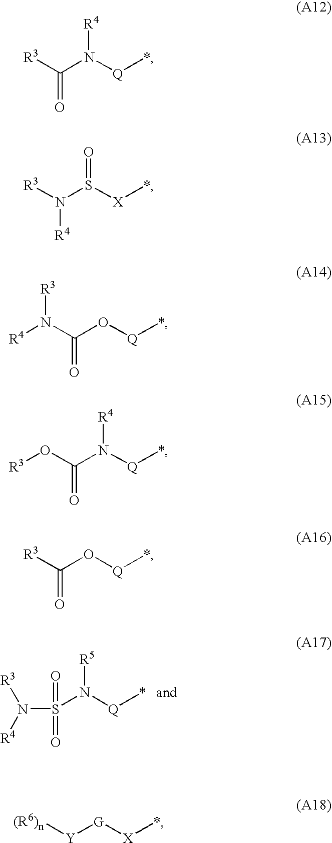

X and Y may be linked to the same or different atoms of G.

X may denote a bond or an optionally substituted group selected from among C₁-C₇-alkylene, C₃-C₇-alkenylene and C₃-C₇-alkynylene, preferably a bond, methyl, ethyl and propyl.

X together with R¹, R³ or R⁴ forms a C₁-C₇-alkylene bridge, preferably a 5- or 6-membered heterocyclic group with R³ or R⁴, particularly preferably a piperidinone or pyrrolidinone—ring with R³ or R⁴ ;which may optionally be substituted.

Y may represent a bond or optionally substituted C₁-C₄-alkylene;preferably a bond or methylene or ethylene.

Q may denote an optionally substituted group selected from among C₁-C₇-alkylene, C₃-C₇-alkenylene and C₃-C₇-alkynylene;preferably optionally substituted C₁-C₃-alkylene, particularly preferably ethyl and propyl. Q may form together with R¹, R³ or R⁴ a C₁-C₇-alkylene bridge.

The substituents R³, R⁴, R⁵ which may be identical or different, may denote hydrogen or an optionally substituted group selected from among C₁-C₈-alkyl, C₃-C₈-cycloalkyl, C₂-C₆-haloalkyl, C₁-C₄-alkyl-C₃-C₈-cycloalkyl, C₃-C₈-cycloalkyl-C₁-C₄-alkyl, NR⁷R⁸, NR⁷R⁸—C₁-C₄-alkyl, C₁-C₄-alkoxy, C₁-C₄-alkoxy-C₁-C₁₄-aryl and C₅-C₁₀-heteroaryl; preferably hydrogen, or an optionally substituted group selected from among C₁-C₄-alkyl, C₁-C₄-alkoxy and C₃-C₆-cycloalkyl, particularly preferably hydrogen, methyl, methoxy, ethoxy, butyloxy and cyclopropyl.

Any two of the substituents R³, R⁴, R⁵ may together form an optionally substituted five-, six- or seven-membered ring, preferably a 5- or 6-membered ring, consisting of carbon atoms and optionally 1-2 heteroatoms, selected from among oxygen, sulphur and nitrogen; preferably oxygen or nitrogen. Preferably the group NR³R⁴ denotes pyrrolidinone or dihydroimidazolidinone.

The substituents R³, R⁴, R⁵ or the ring formed from them may preferably be substituted by one or more of the groups, which may be identical or different, selected from among halogen, NH₂, OH, CN, NR⁹R¹⁰, —NH(CO)—C₁-C₄-alkyl and MeO.

G may denote a saturated, partially saturated or unsaturated ring system consisting of 3-10 C atoms, wherein optionally up to 4 C atoms are replaced by heteroatoms selected from among nitrogen, oxygen and sulphur. Preferably G may denote a saturated, partially saturated or unsaturated ring system consisting of 3-8 C atoms, particularly preferably 5-6 C atoms, wherein optionally up to 6 C atoms, particularly preferably up to 4 C atoms are replaced by heteroatoms selected from among nitrogen, oxygen and sulphur. Particularly preferably G denotes a ring system selected from among cyclohexyl, phenyl, pyrrolidine, piperazine, pyrazole, pyridine, imidazole, thiazole, triazole, oxazole, oxadiazole, tetrazole, benzimidazole, benzopyrrole and dihydro-benzo[1,4]dioxine.

The substituent R⁶, which may be identical or different, may denote hydrogen or an optionally substituted group selected from among C₁-C₈-alkyl, C₃-C₈-cycloalkyl, C₂-C₆-haloalkyl, C₆-C₁₄-aryl, C₅-C₁₀-heteroaryl, C₃-C₈-heterocycloalkyl, preferably hydrogen, or an optionally substituted group selected from among ═O, C₁-C₄-alkyl, C₃-C₆-cycloalkyl, C₆-C₁₄-aryl, C₅-C₆-heterocycloalkyl, and C₅-C₆-heteroaryl, particularly preferably hydrogen or an optionally substituted group selected from among C₁-C₄-alkyl, C₃-C₆-cycloalkyl, C₅-C₆-heterocycloalkyl, C₅-C₆-heteroaryl and phenyl, or

a group selected from among ═O, NR⁷R⁸, OR⁷, —CO—C₁-C₃-alkyl-NR⁷R⁸—O—C₁-C₃-alkyl-NR⁷R⁸, CON R⁷R⁸, NR⁷COR⁸, NR⁷(CO)OR⁸, —CO—C₁-C₃-alkyl-NR⁷(CO)OR⁸, —O(CO)NR⁷R⁸, NR⁷(CO)NR⁸R⁹, NR⁷(CO)OR⁸, (CO)OR⁷, —O(CO)R⁷, COR⁷, (SO)R⁷, (SO₂)R⁷, (SO₂)NR⁷R⁸, NR⁷(SO₂)R⁸, NR⁷(SO₂)NR⁸R⁹, CN and halogen; preferably a group selected from among ═O, NR⁷R⁸, OR⁷, —CO—C₁-C₃-alkyl-NR⁷R⁸, CONR⁷R⁸, NR⁷(CO)OR⁸, NR⁷COR⁸, —CO—C₁-C₃-alkyl-NR⁷(CO)OR⁸, NR⁷(CO)NR⁸R⁹, NR⁷(CO)OR⁸, (CO)OR⁷, COR⁷, (SO₂)R⁷and CN.

The substituent R⁶ may preferably be substituted by one or more of the groups, which may be identical or different, selected from among, NH₂, NHMe, NMe₂, OH, OMe, CN and C₁-C₆-alkyl, —(CO)O—C₁-C₆-alkyl.

n denotes 1, 2 or 3, preferably 1 or 2, particularly preferably 1.

The substituents R⁷, R⁸, R⁹ which may be identical or different, may denote hydrogen or an optionally substituted group selected from among C₁-C₈-alkyl, C₃-C₈-cycloalkyl, C₁-C₆-haloalkyl, C₁-C₄-alkyl-C₃-C₈-cycloalkyl, C₃-C₈-cycloalkyl-C₁-C₃-alkyl, C₆-C₁₄-aryl, C₁-C₄-alkyl-C₆-C₁₄-aryl, C₆-C₁₄-aryl-C₁-C₄alkyl, C₃-C₈-heterocycloalkyl, C₁-C₅-alkyl-C₃-C₈-heterocycloalkyl, C₃-C₈-heterocycloalkyl-C₁-C₄-alkyl, C₁-C₄-alkyl(CO)— and C₁-C₄-alkyl-O(CO), preferably C₁-C₄-alkyl, C₁-C₂-haloalkyl, C₁-C₄-alkyl-C₃-C₈-cycloalkyl, C₃-C₈-cycloalkyl-C₁-C₃-alkyl, phenyl, C₁-C₄-alkyl-C₆-C₁₄-aryl, C₃-C₈-heterocycloalkyl, C₁-C₅-alkyl-C₃-C₈-heterocycloalkyl, C₁-C₄-alkyl(CO)— and C₁-C₄-alkyl-O(CO), particularly preferably C₁-C₅-alkyl, C₁-C₄-alkyl-C₆-C₁₄-aryl, C₃-C₆-heterocycloalkyl and C₁-C₅-alkyl-C₃-C₈-heterocycloalkyl,

or in each case two of the substituents R⁷, R⁸, R⁹ together form an optionally substituted five-, six- or seven-membered ring, consisting of carbon atoms and optionally 1-2 heteroatoms, selected from among oxygen, sulphur and nitrogen; preferably an optionally substituted five- or six-membered ring, consisting of carbon atoms and optionally 1-2 heteroatoms, selected from among oxygen and nitrogen; particularly preferably nitrogen,

The substituents R⁷, R⁸, R⁹ or the ring system formed therefrom may preferably be substituted by one or more of the groups, which may be identical or different, selected from among halogen, NH₂, OH, CN, OMe, NHMe, NMe₂, C₁-C₆-alkyl and (CO)OC₁-C₆-alkyl.

Preparation Processes

The compounds of general formula (I) may be prepared according to the following synthesis scheme (Diagrams 1-4), wherein the substituents of general formula (I) have the meanings given above. These processes are intended to illustrate the invention without restricting it to their content.

The compounds of general formula (I) may be prepared according to the following nach folgendem synthesis scheme (Diagrams 1-7), wherein the substituents of general formula (I) have the meanings given above. These processes are intended to illustrate the invention without restricting it to their content.

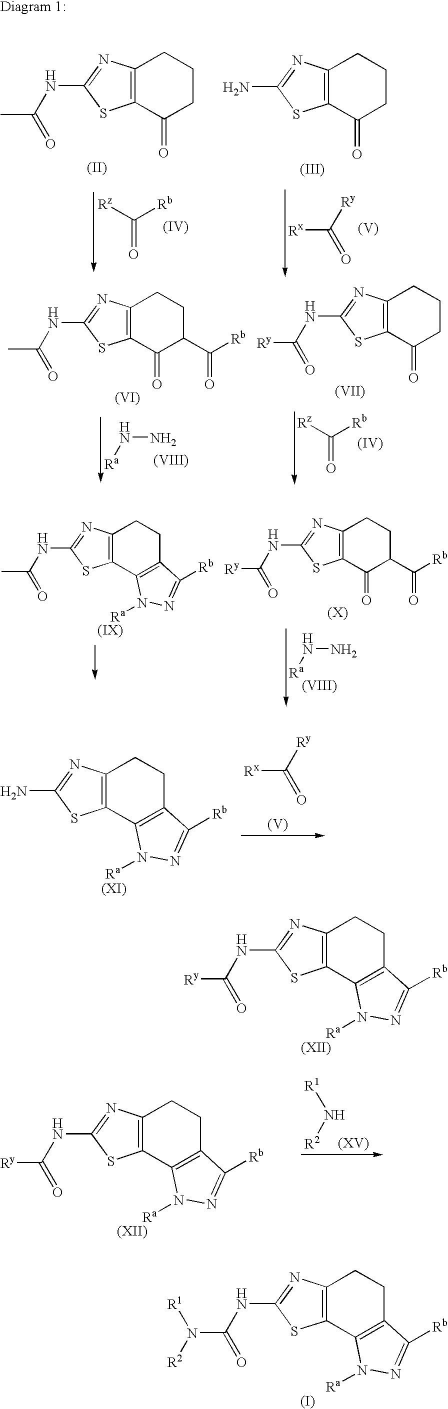

Intermediate compounds of general formula (XII) may be obtained by two different routes, which are described hereinafter:

The intermediate compound (VI) may be obtained by deprotonation of the intermediate compound (II) with a suitable base, selected from, for example, but not restricted to the group comprising sodium methoxide, sodium ethoxide, lithium hexamethylsilazide, sodium hydride and subsequent reaction with a suitable acylating reagent (IV). Rb has the meanings given hereinbefore. Rz is a suitable leaving group selected from, for example, but not restricted to the group comprising halogen, S-alkyl, S-aryl, O-alkylsulphonyl, O-arylsulphonyl, O-alkyl, imizazole, O-hetaryl, O-acyl, O-aryl, wherein O-aryl may optionally be substituted by suitable electron-attracting groups (e.g. nitro). Intermediate compound (IX) is obtained by reaction with a suitable hydrazine (VIII) or one of its salts. Ra has the meanings given hereinbefore. The compound thus obtained is then converted into the free aminothiazole (XI) by cleaving the acetyl group (e.g. by acidic or basic saponification or reaction with hydrazine hydrate). The activated intermediate compound (XII) may be obtained by reaction of the aminothiazole (XI) with a reagent of general formula (V). Alternatively the intermediate compound (III) may be reacted with the reagent (V) described hereinbefore to obtain the compound (VII). Deprotonation of this compound with one of the suitable bases described hereinbefore and acylation with the acylating reagent of general formula (IV) described hereinbefore leads to the intermediate compound (X). This can be converted into the compound of formula (XII) by reaction with the hydrazine (VIII) described hereinbefore or one of the salts thereof.

The compounds of general formula (XII) can be converted by reaction with an amine of general formula (XV) into the compounds of formula (I). R¹ and R² have the meanings described hereinbefore.

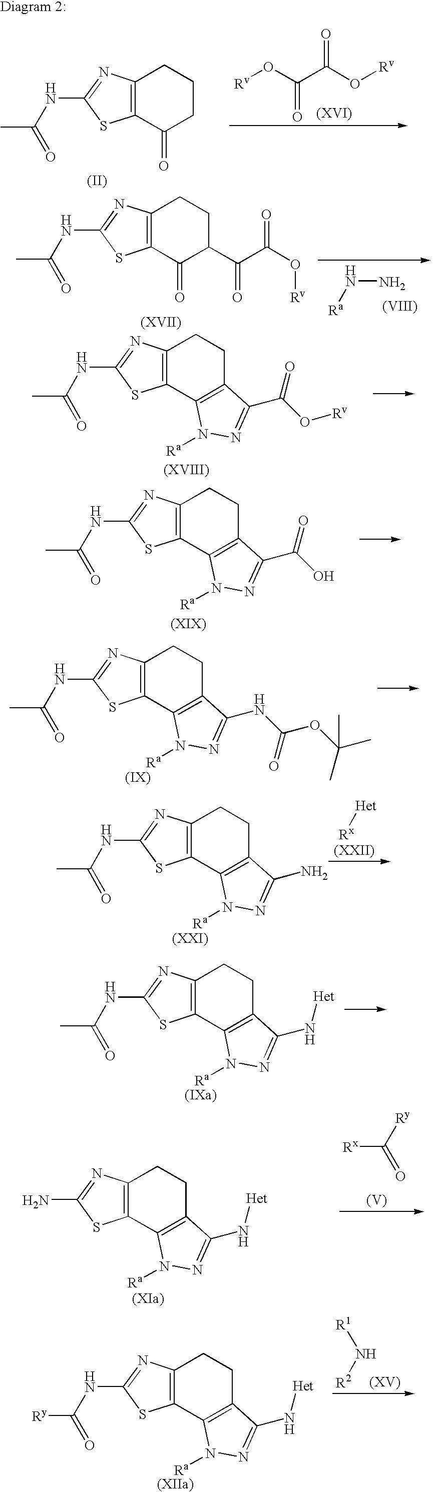

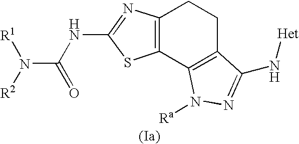

Compounds of general formula (XVII) may be obtained by deprotonation of the intermediate compound (II) with a suitable base analogously to the reaction described in Diagram 1 and subsequent reaction with a reagent of general formula (XVI). Rv denotes an alkyl group. Reaction of this intermediate compound with a hydrazine of formula (VIII) described hereinbefore or one of the salts thereof leads to compounds of general formula (XVIII). Saponification of the ester function yields the carboxylic acid (XIX). Conversion of the carboxylic acid into a carboxylic acid azide and subsequent thermal rearrangement in the presence of tert.-butanol yields intermediate compound (XX). Subsequent cleaving of the BOC-protective group yields the free aminopyrazole (XXI). This aminopyrazole can be converted with reagents of general formula (XXII) into compounds of type (IXa). Rx is a suitable leaving group selected from, for example, but not restricted to the group comprising halogen, S-alkyl, S-aryl, O-alkylsulphonyl, O-arylsulphonyl, O-alkyl, imidazole, O-hetaryl, O-acyl, O-aryl, wherein O-aryl may optionally be substituted by suitable electron-attracting groups (e.g. nitro). Het represents a suitable heteroaromatic ring. The further reactions are carried out analogously to Diagram 1: cleaving of the N-acetyl group to obtain the aminopyrazole (XIa), reaction with reagent (V) to obtain the compound (XIIa) and final reaction of (XIIa) with suitable amines of formula (XV) described hereinbefore yield the compounds of formula (Ia).

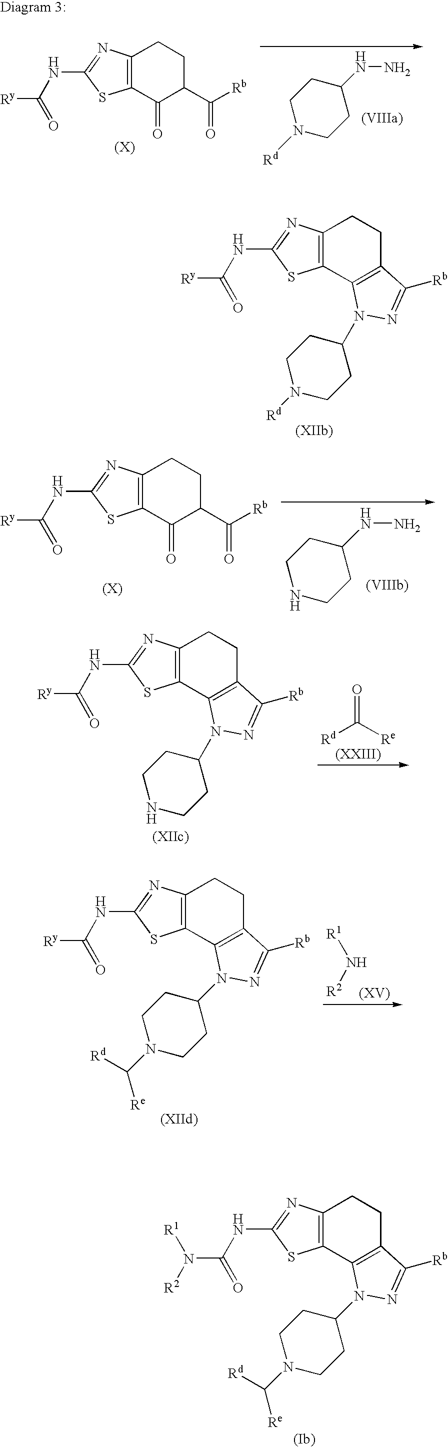

The reaction of intermediate compounds of general formula (X) with hydrazines of general formula (VIIIa) yields compounds of general formula (XIIb). R^{d} is selected from among H, C₁-C₆-alkyl. Where R^{d}═H compounds of the general type (XIIc) 10 are obtained, which can be converted by reductive amination of the ketones or aldehydes (XXIII) into compounds of general formula (XIId). R^{d} and R^{e} which may be identical or different are selected from among H, C₁-C₆-alkyl and may optionally together also form a 3-8-membered ring. The reaction of intermediate compounds of the type (XIIb), (XIIc) or (XIId) with suitable amines of formula (XV) described hereinbefore leads to compounds of general formula (I), illustrated by way of example for the reaction of (XIId) to (Ib).

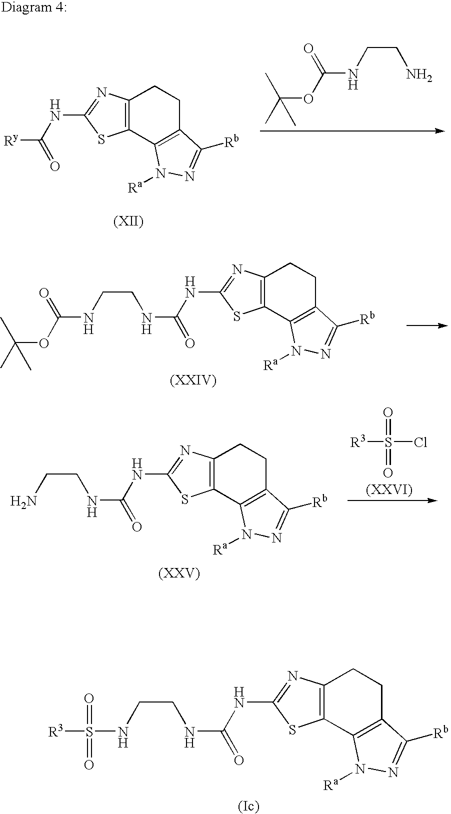

By reacting intermediate compounds of general formula (XII) with tert-butyl (2-amino-ethyl)-carbamate intermediate compounds of type (XXIV) are obtained which, after the cleaving of the BOC-protective group to obtain compounds of formula (XXV), can be reacted with reagents of general formula (XXVI) to obtain compounds of formula (Ic). R³ has the meanings described hereinbefore.

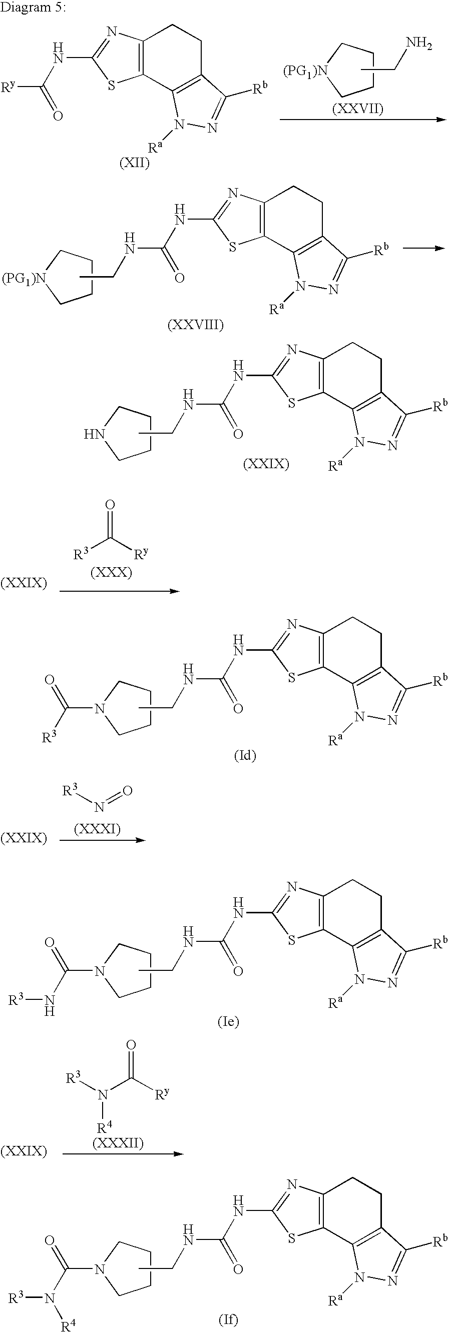

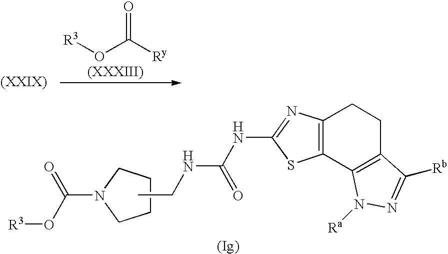

By reacting the intermediate compound (XII) with a reagent of formula (XXVII) compounds of general formula (XXVIII) are obtained. The reagent (XXVII) may be used as one of the two possible regioisomers. Each of these regioisomers can be used in each case as one of the the two possible enantiomers or as the racemate. PG1 is a suitable nitrogen-protective group selected from, for example, but not restricted to the group comprising alkylcarbonyl-(carbamate), benzyl-(optionally substituted e.g. p-methoxybenzyl). After the cleaving of the protective group PG1 the intermediate compound (XXIX) can be obtained. Reaction of this intermediate compound with reagents of type (XXX), (XXXI), (XXXII) or (XXXIII) leads to the compounds (Id), (Ie), (If) or (Ig). R³ and R⁴ have the meanings described hereinbefore. R^{y} is a suitable leaving group selected from, for example, but not restricted to the group comprising halogen, S-alkyl, S-aryl, O-alkylsulphonyl, O-arylsulphonyl, O-alkyl, imidazol, O-hetaryl, O-acyl, O-aryl, wherein O-aryl may optionally be substituted by suitable electron-attracting groups (e.g. nitro).

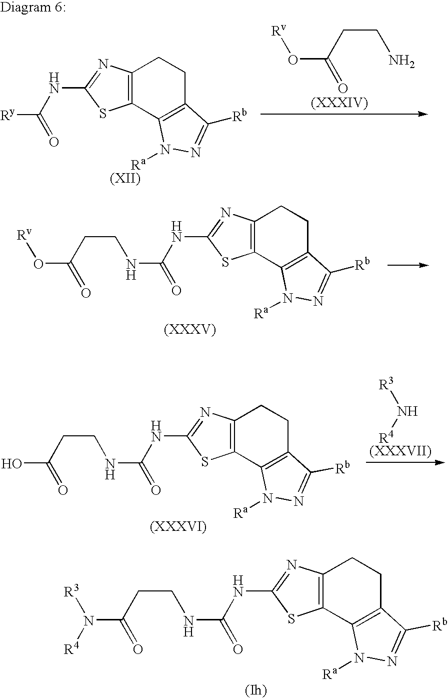

By reacting the intermediate compound (XII) with the amino acid ester (XXXIV), after saponification of the ester function of (XXXV), it is possible to obtain the carboxylic acid (XXXVI), which after suitable activation by methods known from the literature can be reacted with amines of general formula (XXXVII). Compounds of general formula (Ih) are obtained. R^{v}, R³ and R⁴ have the meanings described hereinbefore.

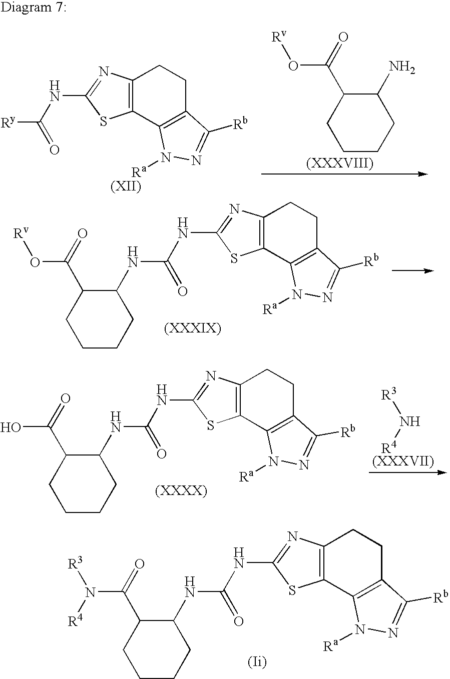

By reacting the intermediate compound (XII) with the amino acid ester (XXXVIII), after saponification of the ester function of (XXXIX), the carboxylic acid (XXXX) may be obtained, which after suitable activation by methods known from the literature, can be reacted with amines of formula (XXXVII) described hereinbefore. Compounds of general formula (Ii) are obtained. R^{v}, R³ and R⁴ have the meanings described hereinbefore. The reagent (XXXVIII) may be used in the form of one of the possible stereoisomers or as a mixture of two or more of these stereoisomers.

The new compounds of general formula (I) may be prepared analogously to to the Examples that follow. The Examples described hereinafter are intended to illustrate the invention without restricting it.
Synthesis of the Reagents:

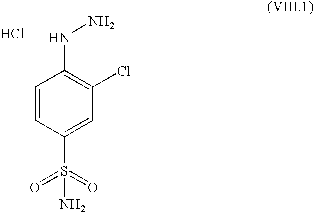

### 3-chloro-4-fluoro-benzenesulphonamide

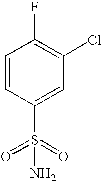

6.21 ml (42 mmol) 3-chloro-4-fluorobenzenesulphonic acid chloride are placed in 20 ml dioxane and cooled to 5° C. 85 ml (1.14 mmol) ammonia solution in water (25%) are added dropwise, the mixture is stirred for 6 hours at 5° C. and for 16 hours at ambient temperature. Then it is evaporated down until a precipitate settles out. This is suction filtered, washed with water and dried.

Yield: 8.30 g (94% of theoretical)

### 3-chloro-4-hydrazino-benzenesulphonamide

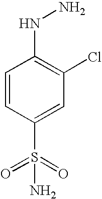

8.24 g (39 mmol) 3-chloro-4-fluoro-benzenesulphonamide are dissolved in 200 ml acetonitrile, 260 ml (260 mmol) hydrazine (1 molar solution in tetrahydrofuran) are added. The reaction mixture is stirred for 6 hours at reflux temperature and for 16 hours at ambient temperature. After cooling it is evaporated down, the residue is stirred with water for 0.2 hours. The precipitate is suction filtered, washed and dried.

Yield: 6.99 g (64% of theoretical)

### 3-chloro-4-hydrazino-benzenesulphonamide hydrochloride (VIII.1)

7.00 g (25.26 mmol) 3-chloro-4-hydrazino-benzenesulphonamide are placed in 500 ml of ethanol, and 12.33 ml (24.66 mmol) 2molar ethereal hydrochloric acid are added dropwise. The mixture is stirred for 0.5 hours at ambient temperature, then evaporated down. The residue is extracted with acetonitrile.

Yield: 6.10 g (94% of theoretical)

### Imidazol-1-yl-cyclopropyl-methanone (IV.1)

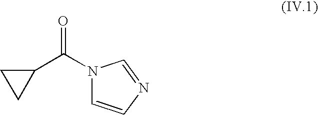

75 g (0.46 mol) carbonyidiimidazole and 30.0 g (0.35 mol) cyclopropanecarboxylic acid are stirred for 20 h at RT. Then the reaction mixture is washed twice with 200 mL saline solution, the organic phase is dried and the solvent is eliminated i. vac.

Yield: 45.5 g (96%).

### Imidazol-1-yl-pyridin-3-yl-methanone (IV.2)

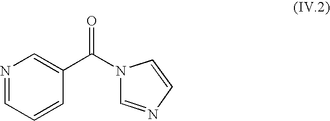

39.56 g (321.34 mmol) nicotinic acid and 53.72 g (321 mmol) carbonyldiimidazole are stirred in 160 ml dichloromethane for 16 hours at ambient temperature. Then the reaction mixture is extracted with water, the organic phase is dried and evaporated to dryness.

Yield: 40.52 g (73% of theoretical)

### Imidazol-1-yl-pyrimidin-5-yl-methanone (IV.3)

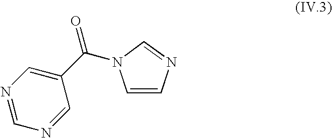

### Pyrimidine-5-carboxylic acid

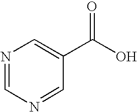

19.50 ml (149 mmol) ethyl pyrimidine-5-carboxylate are shaken in 40 ml of 4 molar sodium hydroxide solution for 0.1 hours at ambient temperature, then 40 ml of 4 molar hydrochloric acid solution are added. The precipitate formed is suction filtered, washed with petroleum ether and dried.

Yield: 14.80 g (80% of theoretical)

### Pyrimidine-5-carbonyl chloride hydrochloride

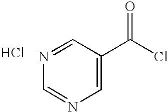

7.50 g (60 mmol) pyrimidine-5-carboxylic acid are stirred in 50 ml of thionyl chloride and 0.5 ml dimethylformamide for 4 hours at 70° C. The reaction mixture is evaporated to dryness and re-evaporated several times with toluene.

Yield: 7.80 g (72% of theoretical)

### Imidazol-1-yl-pyrimidin-5-yl-methanone (IV.3)

5.02 g (74 mmol) imidazole are dissolved in 150 ml dichloromethane and cooled to −5° C. 4.40 g (25 mmol) pyrimidine-5-carbonyl chloride hydrochloride dissolved in 50 ml dichloromethane and 2 ml dimethylacetamide are added dropwise. The reaction mixture is stirred for 2.5 hours at ambient temperature. After cooling it is extracted with water. The organic phase is dried and evaporated to dryness.

Yield: 2.50 g (51 % of theoretical)

### Synthesis of the reagent (2S)-2-amino-1-piperidin-1-yl-propan-1-one hydrochloride (XV.1)

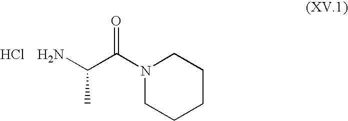

### Tert-butyl (2S)-(1-methyl-2-oxo-2-piperidin-1-yl-ethyl)-carbamate

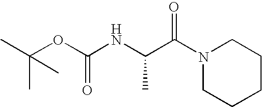

3.50 g (18.50 mmol) (2S)-2-tert-butoxycarbonylamino-propionic acid, 1.83 ml (18.48 mmol) piperidine, 5.70 g (18.26 mmol) O-(1H-benzotriazol-1-yl)-N,N,N′,N′-tetramethyluronium tetrafluoroborate (TBTU) and 6.30 ml (37.04 mmol) diisopropylethylamine are stirred in 10 ml dichloromethane for 16 hours at ambient temperature. Then the mixture is diluted with dichloromethane and extracted with sodium hydrogen carbonate solution, sodium hydrogen sulphate solution and water. The organic phase is dried and evaporated to dryness.

Yield: 5.19 g

### (2S)-2-amino-1-piperidin-1-yl-propan-1-one hydrochloride (XV.1)

5.19 g (20.25 mmol) tert-butyl (2S)-(1-methyl-2-oxo-2-piperidin-1-yl-ethyl)-carbamate are stirred in 15 ml of 4 molar dioxanic hydrochloric acid for 16 hours at ambient temperature. Then the mixture is evaporated down and the residue is crystallised with ethyl acetate.

Yield: 2.83 g (69% of theoretical)

Reagents (XV.2)-(XV.8) may be obtained analogously using the appropriate enantiomers of 2-tert-butoxycarbonylamino-propionic acid and the corresponding amines:

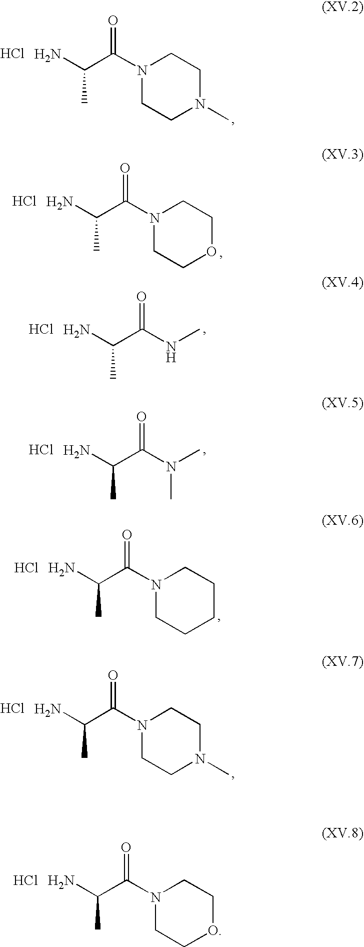

### Synthesis of the Reagent 3-isopropyl-benzylamine hydrochloride (XV.9)

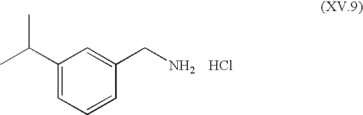

### 3-isopropyl-benzonitrile

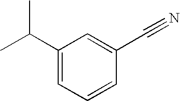

5.15 g (25.87 mmol) m-bromo-isopropyl-benzene and 2.69 g (30.04 mmol) copper cyanide are stirred in 2.50 ml of pyridine for 24 hours at 180° C. Then 15 ml of water, 15 ml of toluene and 15 ml conc. ammonia solution are added, then the mixture is extracted. The organic phase is dried and evaporated to dryness.

Yield: 5.00 g (100% of theoretical)

### 3-isopropyl-benzylamine hydrochloride (XV.9)

5.00 g (34.43 mmol) 3-isopropyl-benzonitrile and 5.00 g Raney nickel are hydrogenated in 500 ml of methanolic ammonia solution for 8 hours at ambient temperature under a pressure of 50 psi. After the catalyst has been filtered off the mixture is evaporated down and the residue is precipitated as the hydrochloride.

Yield: 2.90 g (45% of theoretical)

### Synthesis of the Reagent (XV.10)

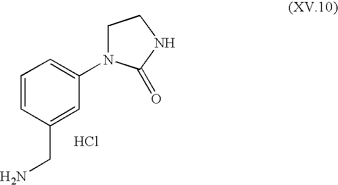

### 1-(2-chloro-ethyl)-3-(3-cyano-phenyl)-urea

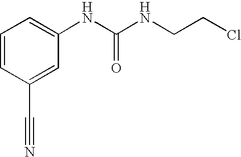

65.00 g (550 mmol) 3-amino-benzonitrile are dissolved in 450 ml dioxane, 56 ml (660 mmol) 1-chloro-2-isocyanato-ethane dissolved in 60 ml dioxane are added dropwise. The reaction mixture is stirred for 3 hours at 60° C. and for 16 hours at ambient temperature. Then the precipitate is suction filtered, washed with diethyl ether and dried.

Yield: 110.00 g (90% of theoretical)

mp: 138°-139° C.

### 3-(2-oxo-imidazolidin-1-yl)-benzonitrile

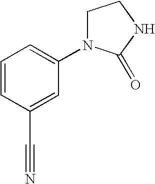

110.00 g (490 mmol) 1-(2-chloro-ethyl)-3-(3-cyano-phenyl)-urea are dissolved in 2000 ml of ethanol at 50° C. and a solution of 42.00 g (640 mmol) potassium hydroxide in 390 ml of ethanol is added within 1.5 hours. The reaction mixture is stirred for 16 hours at ambient temperature, then the precipitate formed is suction filtered, washed with water and dried.

Yield: 68.00 g (75% of theoretical)

mp: 149°-150° C.

### 1-(3-aminomethyl-phenyl)-imidazolidin-2-one hydrochloride (XV.10)

40.00 g (210 mmol) 3-(2-oxo-imidazolidin-1-yl)-benzonitrile are suspended in 1500 ml of methanol, 53 ml of 37% hydrochloric acid are added. The mixture is hydrogenated for 20 hours at ambient temperature under a pressure of 7 bar with 4.00 g palladium/charcoal. The catalyst is filtered off, the filtrate is concentrated and the precipitate formed is suction filtered, washed with acetone and dried.

Yield: 42.00 g (88% of theoretical)

mp: 238°-239° C.

The reagent (XV.11) may also be prepared analogously

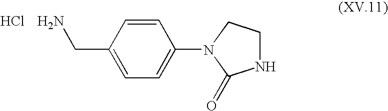

### Synthesis of the Reagent (XV.12)

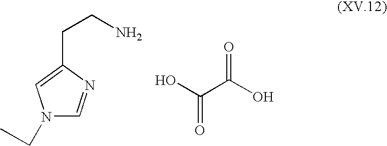

### 7,8-dihydro-6H-imidazo[1,5-c]pyrimidin-5-one

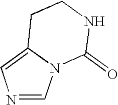

50.00 g (450 mmol) histamine are dissolved in 1500 ml dimethylformamide, 73.87 g (450 mmol) carbonyldiimidazole are added. The reaction mixture is stirred for 5 hours at 70° C. and for 16 hours at ambient temperature. Then it is evaporated down, the residue is extracted hot from acetonitrile.

Yield: 53.73 g (87% of theoretical)

### 2-ethyl-5-oxo-5,6,7,8-tetrahydro-imidazo[1,5-c]pyrimidin-2-ium bromide

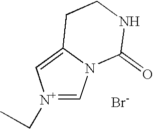

1.00 g (7 mmol) 7,8-dihydro-6H-imidazo[1,5-c]pyrimidin-5-one and 1.57 ml (21 mmol) ethylbromide are stirred in 12 ml acetonitrile for 16 hours at 80° C. After cooling the suspension is suction filtered, washed and dried.

Yield: 1.40 g (78% of theoretical)

### 2-(1-ethyl-1H-imidazol-4-yl)-ethylamine oxalate (XV.12)

1.16 g (5 mmol) 2-ethyl-5-oxo-5,6,7,8-tetrahydro-imidazo[1,5-c]pyrimidin-2-ium bromide are refluxed in 7 ml (14 mmol) 2 molar hydrochloric acid for 16 hours with stirring. Then the mixture is evaporated down, the residue is recrystallised acetonitrile/ethanol. The highly hygroscopic crystals obtained are made neutral and evaporated down. The residue is precipitated as the oxalate and recrystallised from ethanol.

Yield: 1.00 g (93% of theoretical)

### Synthesis of the Reagent (XV.13)

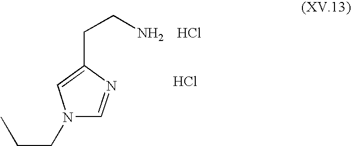

### 5-oxo-2-propyl-5,6,7,8-tetrahydro-imidazo[1,5-c]pyrimidin-2-ium bromide

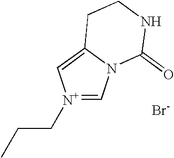

2.00 g (15 mmol) 7,8-dihydro-6H-imidazo[1,5-c]pyrimidin-5-one and 6.83 mmol) (75 mmol) propylbromide are stirred in 20 ml acetonitrile for 72 hours at 85° C. After cooling the suspension is suction filtered, washed and dried.

Yield: 3.48 g

### 2-(1-propyl-1H-imidazol-4-yl)-ethylamine dihydrochloride (XV.13)

100 mg (0.384 mmol) 5-oxo-2-propyl-5,6,7,8-tetrahydro-imidazo[1,5-c]pyrimidin-2-ium bromide are refluxed in 192 μl (1.15 mmol) 6 molar hydrochloric acid for 16 hours with stirring. Then the solution is lyophilised.

Yield: 81.30 mg (64% of theoretical)

### Synthesis of the Reagent (XV.14)

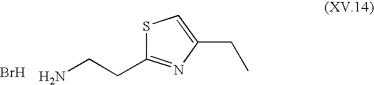

### 2-(4-ethyl-thiazol-2-yl)-ethylamine hydrobromide (XV.14)

2.00 g (9.50 mmol) tert. butyl N(3-amino-3-thioxopropyl)carbamate and 1.58 g (10.45 mmol) 1-bromo-2-butanone are refluxed in 40 ml of ethanol for 16 hours with stirring. The reaction mixture is evaporated down, the residue is purified by chromatography.

Yield: 2.00 g (89% of theoretical)

### Synthesis of the Reagent (XV.15)

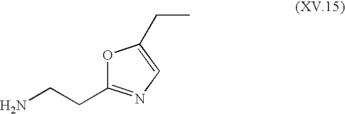

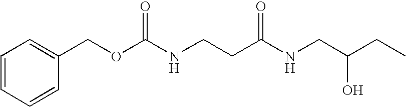

23.20 g (103.93 mmol) 3-benzyloxycarbonylamino-propionic acid, 14.10 g (104.35 mmol) 1-hydroxybenzotriazole, 18.80 ml (135.07 mmol) triethylamine and 21.00 g (135.27 mmol) (ethyl-3-(3-dimethylamino)-propylcarbondiimide hydrochloride (EDAC) are placed in 150 ml dichloromethane, cooled to 0° C. and stirred for 0.75 hours at this temperature. Then 10.50 g (114.26 mmol) 1-amino-2-butanol are added and the mixture is stirred for 2.5 hours at 0°-5° C. The reaction mixture is extracted with water and 1 molar sodium carbonate solution, the organic phase is dried and evaporated to dryness. The residue is extracted again with dichloromethane and sodium carbonate solution.

Yield: 12.30 g (40% of theoretical)

### benzyl [2-(2-oxo-butylcarbamoyl)-ethyl]-carbamate

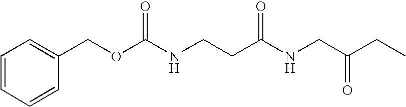

2.20 ml (26.05 mmol) oxalyl chloride are placed in 10 ml dichloromethane, the solution is cooled to −53° C. 2.45 ml (34.49 mmol) dimethylsulphoxide in 5 ml dichloromethane are slowly added dropwise, the mixture is stirred for 0.25 hours, then a solution of 6.30 g (21.40 mmol) benzyl [2-(2-hydroxy-butylcarbamoyl)-ethyl]-carbamate in 30 ml dichloromethane is added. The mixture is stirred for 1.5 hours at −60° C., then 12.60 ml triethylamine is added dropwise. The suspension is stirred for 1 hour at −50° C., then allowed to come up to ambient temperature within 16 hours. The reaction mixture is diluted with dichloromethane, and extracted with 1 molar hydrochloric acid, 1 molar sodium carbonate solution and water. The organic phase is dried and evaporated to dryness.

Yield: 5.82 g (93% of theoretical)

### benzyl [2-(5-ethyl-oxazol-2-yl)-ethyl]-carbamate

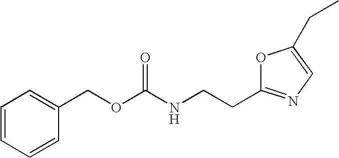

23.07 g (49.60 mmol) PS-triphenylphosphine are suspended in 200 ml dichloromethane, 12.65 g (49.82 mmol) iodine are added. The mixture is stirred for 0.1 hours at ambient temperature, then 13.80 ml (99.28 mmol) triethylamine are added dropwise. 5.80 g (19.84 mmol) benzyl [2-(2-oxo-butylcarbamoyl)-ethyl]-carbamate dissolved in 150 ml dichloromethane are added. The reaction mixture is stirred for 72 hours at ambient temperature, then the precipitate is filtered off. The filtrate is extracted with water, the organic phase is dried and evaporated to dryness.

Yield: 3.35 g (31 % of theoretical)

### 2-(5-ethyl-oxazol-2-yl)-ethylamine (XV.15)

2.86 g (10.43 mmol) benzyl [2-(5-ethyl-oxazol-2-yl)-ethyl]-carbamate are placed in 130 ml of methanol, 0.910 mg 10% palladium/charcoal are added, then the mixture is hydrogenated for 5 hours at ambient temperature under a pressure of 14 psi. Then the catalyst is removed by suction filtering and the solution is evaporated down.

Yield: 1.45 g (99% of theoretical)

### Synthesis of the Reagent (XV.16)

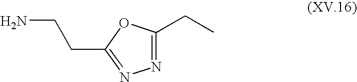

### tert-butyl [3-oxo-3-(N′-propionyl-hydrazino)-propyl]-carbamate

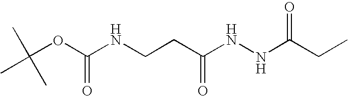

25.00 g (132 mmol) 3-tert-butoxycarbonylamino-propionic acid, 11.45 g (130 mmol) ethanoic hydrzide, 50.91 g (159 mmol) O-(1H-benzotriazol-1-yl)-N,N,N′,N′-tetramethyluronium tetrafluoroborate (TBTU) and 50 ml diisopropylethylamine are stirred in 500 ml of tetrahydrofuran/dichloromethane for 24 hours at ambient temperature. Then the mixture is evaporated down, the residue is extracted with ethyl acetate and 10% potassium hydrogen carbonate solution. The organic phase is dried and evaporated to dryness. The residue is crystallised from isopropylether.

Yield: 3.20 g (9% of theoretical)

### tert-butyl [2-(5-ethyl-[1,3,4]oxadiazol-2-yl)-ethyl]-carbamate

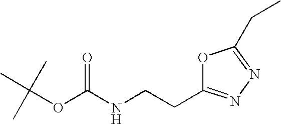

11.49 g (24.70 mmol) PS-triphenylphosphine are placed in 240 ml dichloromethane, 6.27g (24.70 mmol) iodine are added. The mixture is stirred for 0.1 hours at ambient temperature, then 7.00 ml (50.50 mmol) triethylamine are added dropwise. 3.20 g (12.34 mmol) tert-butyl [3-oxo-3-(N′-propionyl-hydrazino)-propyl]-carbamate dissolved in 150 ml dichloromethane are added. The reaction mixture is stirred for 24 hours at ambient temperature, then the precipitate is filtered off. The filtrate is evaporated down and purified by chromatography.

Yield: 2.95 g (99% of theoretical)

### 2-(5-ethyl-[1,3,4]oxadiazol-2-yl)-ethylamine (XV.16)

2.95 g (12.23 mmol) tert-butyl [2-(5-ethyl-[1,3,4]oxadiazol-2-yl)-ethyl]-carbamate and 10 ml trifluoroacetic acid are stirred in 100 ml dichloromethane for 24 hours at ambient temperature. Then the mixture is evaporated down, the residue is made basic and extracted with ethyl acetate. The organic phase is dried and evaporated to dryness.

Yield: 0.410 g (24% of theoretical)

### Synthesis of the Reagent (XV.17)

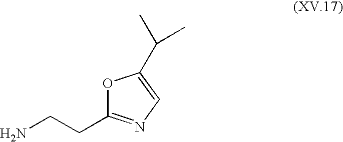

### benzyl [2-(2-hydroxy-3-methyl-butylcarbamoyl)-ethyl]-carbamate

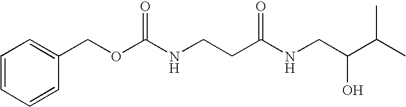

46.00 g (206.07 mmol) 3-benzyloxycarbonylamino-propionic acid, 51.37 g (267.95 mmol) 1-(3-dimethylaminopropyl)-3-ethylcarbodiimide hydrochloride, 27.85 g (206.07 mmol) hydroxybenzotriazole (HOBT) and 37.14 ml (267.95 mmol) triethylamine are placed in 700 ml dichloromethane, the mixture is stirred for 0.5 hours at 0°, then 23.70 g (229.73 mmol) 1-amino-3-methyl-butan-2-ol are added. The reaction mixture is stirred for 16 hours at ambient temperature. Then it is extracted with potassium carbonate solution and dichloromethane. The organic phase is washed with 1 molar sodium hydroxide solution, dried and evaporated to dryness. The residue is stirred with diethyl ether, then recrystallised with acetonitrile.

Yield: 32.40 g (51% of theoretical)

### benzyl [2-(3-methyl-2-oxo-butylcarbamoyl)-ethyl]-carbamate

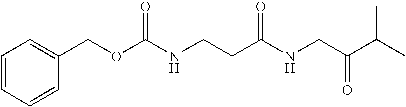

10.81 ml (126.08 mmol) oxalyl chloride are placed in 300 ml dichloromethane, cooled to −70° C. 11.94 ml (168.11 mmol) dimethylsulphoxide are slowly added dropwise. The mixture is stirred for 0.1 hours, then 32.40 g (105.07 mmol) benzyl [2-(2-hydroxy-3-methyl-butylcarbamoyl)-ethyl]-carbamate in 70 ml dichloromethane are added. The mixture is stirred for 1 hour, then 62.48 ml (450.72 mmol) triethylamine are added dropwise. The reaction mixture is stirred for 1.5 hours at −70° C., then slowly allowed to come up to ambient temperature. It is diluted with dichloromethane and washed with 1 molar hydrochloric acid, saturated sodium carbonate solution, water and saturated sodium chloride solution. The organic phase is dried and evaporated to dryness.

Yield: 30.80 g (96% of theoretical)

### benzyl [2-(5-isopropyl-oxazol-2-yl)-ethyl]-carbamate

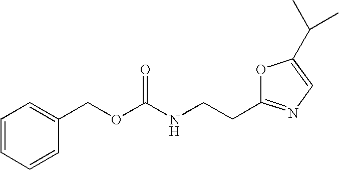

100.00 g (215 mmol) PS-triphenylphosphine are suspended in 1000 ml dichloromethane, 59.92 g (236.06 mmol) iodine are added. The mixture is stirred for 0.1 hours at ambient temperature, then 65.32 ml (470.24 mmol) triethylamine are added dropwise. 28.80 g (94.91 mmol) benzyl [2-(3-methyl-2-oxo-butylcarbamoyl)-ethyl]-carbamate dissolved in 200 ml dichloromethane are added. The reaction mixture is stirred for 16 hours at ambient temperature. As the reaction is incomplete, a further 0.1 eq triphenylphosphine and 0.1 eq iodine are added. It is stirred for 16 hours at ambient temperature, then the precipitate is filtered off. The filtrate is evaporated down, the residue is extracted with water and chloroform, the organic phase is dried and evaporated to dryness. The residue is purified by chromatography.

Yield: 12.50 g (46% of theoretical)

### 2-(5-isopropyl-oxazol-2-yl)-ethylamine (XV.17)

6.50 g (22.54 mmol) benzyl [2-(5-isopropyl-oxazol-2-yl)-ethyl]-carbamate are placed in 130 ml of methanol, 3.50 g palladium/charcoal 10% are added, then the mixture is hydrogenated for 5 hours at ambient temperature under a pressure of 14 psi. Then the catalyst is removed by suction filtering, the solution is evaporated down. The residue is extracted with dichloromethane and potassium carbonate solution, the organic phase is dried and evaporated to dryness.

Yield: 3.20 g (92% of theoretical)

### Synthesis of the Reagent (XV.18)

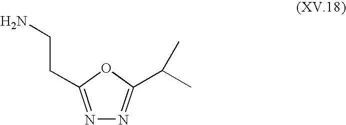

### tert-butyl [3-(N′-isobutyryl-hydrazino)-3-oxo-propyl]-carbamate

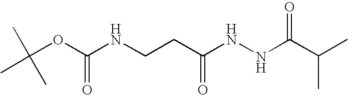

25.00 g (132 mmol) 3-tert-butoxycarbonylamino-propionic acid, 13.50 g (132 mmol) isobutyric acid hydrazide, 50.91 g (159 mmol) O-(1H-benzotriazol-1-yl)-N,N,N′,N′-tetramethyluronium tetrafluoroborate (TBTU) and 50 ml diisopropylethylamine are stirred in 500 ml of tetrahydrofuran/dichloromethane for 24 hours at ambient temperature. Then the mixture is evaporated down, the residue is extracted with ethyl acetate and 10% potassium hydrogen carbonate solution. The organic phase is dried and evaporated to dryness. The residue is crystallised from toluene/isopropylether.

Yield: 16.55 g (46% of theoretical)

### tert-butyl [2-(5-isopropyl-[1,3,4]oxadiazol-2-yl)-ethyl]-carbamate

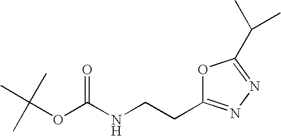

20.00 g (43.00 mmol) PS-triphenylphosphine are placed in 240 ml dichloromethane, 10.88g (42.87 mmol) iodine are added. The mixture is stirred for 0.1 hours at ambient temperature, then 12.10 ml (87.29 mmol) triethylamine are added dropwise. 5.83 g (21.33 mmol) tert-butyl [3-(N′-isobutyryl-hydrazino)-3-oxo-propyl]-carbamate dissolved in 150 ml dichloromethane are added. The reaction mixture is stirred for 24 hours at ambient temperature, then the precipitate is filtered off. The filtrate is evaporated down and purified by chromatography.

Yield: 5.40 g (99% of theoretical)

### 2-(5-isopropyl-[1,3,4]oxadiazol-2-yl)-ethylamine (XV.18)

4.00 g (15.67 mmol) tert-butyl [2-(5-isopropyl-[1,3,4]oxadiazol-2-yl)-ethyl]-carbamate and 20 ml trifluoroacetic acid are stirred in 200 ml dichloromethane for 24 hours at ambient temperature. Then the mixture is evaporated down, the residue is made basic and extracted with ethyl acetate. The organic phase is dried and evaporated to dryness.

Yield: 1.440 g (59% of theoretical)

### Synthesis of the Reagent (XV.19)

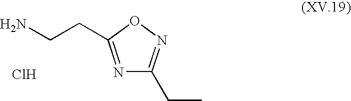

### methyl 3-tert-butoxycarbonylamino-propionate

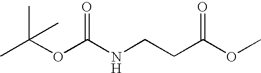

9.90 g (70.93 mmol) β-alaninemethylester hydrochloride are placed in 200 ml acetonitrile, 10 ml (72.14 mmol) triethylamine are added. The mixture is stirred for 0.3 hours at ambient temperature, first 15.48 g (70.93 mmol) Boc-anhydride, then 1.65 g (7.09 mmol) zirconium(IV)chloride are added. The reaction mixture is stirred for 2 hours at ambient temperature, then evaporated down. The residue is extracted with ethyl acetate and water. The organic phase is dried and evaporated to dryness.

Yield: 12.50 g (87% of theoretical)

### N-hydroxy-propionamidine

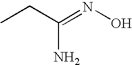

8.00 g (57.88mmol) potassium carbonate are dissolved in 25 ml of water, 80 ml of ethanol, 4.00 g (57.56 mmol) hydroxylamine and 4.11 ml (57.56 mmol) propionitrile are added. The reaction mixture is stirred for 18 hours at ambient temperature, then evaporated down and concentrated by rotary evaporation with toluene. The residue is mixed with ethanol, suction filtered and the filtrate is evaporated to dryness.

Yield: 3.70 g (73% of theoretical)

### tert-butyl [2-(3-ethyl-[1,2,4]oxadiazol-5-yl)-ethyl]-carbamate

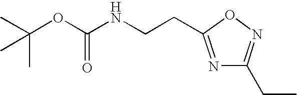

2.00 g (22.70 mmol) N-hydroxy-propionamidine are placed in 10 ml dimethylformamide and molecular sieve. 0.999 g (24.97 mmol) sodium hydride (60% in mineral oil) are added. The mixture is stirred for 0.1 hours at 50° C., then 5.00 g (24.60 mmol) methyl 3-tert-butoxycarbonylamino-propionate in 20 ml dimethylformamide are added. The reaction mixture is stirred for 3 hours at 50° C. After cooling 15 ml of water are added, and the mixture is suction filtered through Celite. The 2 phases of the filtrate are separated, the organic phase is evaporated down. The residue is purified by chromatography.

Yield: 2.05 g (37% of theoretical)

### 2-(3-ethyl-[1,2,4]oxadiazol-5-yl)-ethylamine hydrochloride (XV.19)

2.05 g (8.50 mmol) tert-butyl [2-(3-ethyl-[1,2,4]oxadiazol-5-yl)-ethyl]-carbamate are placed in 20 ml dichloromethane, 40 ml of 1 molar ethereal hydrochloric acid are added. The reaction mixture is stirred for 16 hours at ambient temperature and 4 hours at 40° C. After the addition of a further 10 ml ethereal hydrochloric acid the mixture is stirred for another 72 hours at ambient temperature. The suspension is evaporated down.

Yield: 1.50 g (99% of theoretical)

### Synthesis of the Reagent (XV.20)

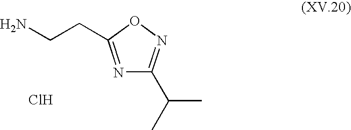

N-hydroxy-isobutyramidine

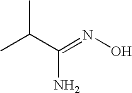

6.00 g (43.41 mmol) potassium carbonate are dissolved in 19 ml of water, 60 ml of ethanol, 3.00 g (43.17 mmol) hydroxylamine and 3.95 ml (43.44 mmol) isobutyronitrile are added. The reaction mixture is stirred for 18 hours at ambient temperature, then evaporated down and re-evaporated with toluene. The residue is mixed with ethanol, suction filtered and the filtrate is evaporated to dryness.

Yield: 3.70 g (84% of theoretical)

### tert-butyl [2-(3-isopropyl-[1,2,4]oxadiazol-5-yl)-ethyl]-carbamate

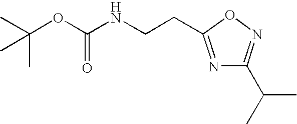

2.20 g (21.54 mmol) N-hydroxy-isobutyramidine are placed in 10 ml dimethylformamide and molecular sieve. 0.948 g (23.69 mmol) sodium hydride (60% in mineral oil) are added. The mixture is stirred for 0.1 hours at 50° C., then 6.20 g (30.51 mmol) methyl 3-tert-butoxycarbonylamino-propionate in 20 ml dimethylformamide are added. The reaction mixture is stirred for 3 hours at 50° C. After cooling 15 ml of water are added and the mixture is suction filtered through Celite. The 2 phases of the filtrate are separated, the aqueous phase is extracted with ethyl acetate, the combined organic phase evaporated down. The residue is purified by chromatography.

Yield: 0.900 g (16% of theoretical)

### 2-(3-isopropyl-[1,2,4]oxadiazol-5-yl)-ethylamine hydrochloride (XV.20)

900 mg (3.53 mmol) tert-butyl [2-(3-isopropyl-[1,2,4]oxadiazol-5-yl)-ethyl]-carbamate are placed in 10 ml dichloromethane, 20 ml 1 molar ethereal hydrochloric acid are added. The reaction mixture is stirred for 16 hours at ambient temperature. After the addition of a further 10 ml ethereal hydrochloric acid the mixture is stirred for another 72 hours at ambient temperature and 4 hours at 40° C. The suspension is evaporated down. The residue is dissolved in acetone, mixed with diethyl ether and suction filtered.

Yield: 530 mg (78% of theoretical)

### Synthesis of the Reagent (XV.21)

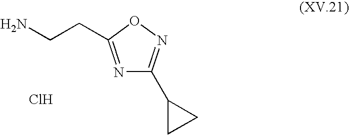

### Ethyl 3-tert-butoxycarbonylamino-propionate

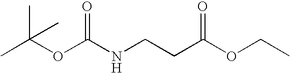

5.00 g (32.55 mmol) β-alanineethylester hydrochloride are placed in 100 ml acetonitrile, 4.75 ml (34.27 mmol) triethylamine are added. The mixture is stirred for 0.3 hours at ambient temperature, first 7.30 g (33.45 mmol) Boc-anhydride, then 0.759 g (3.26 mmol) zirconium(IV)chloride are added. The reaction mixture is stirred for 2 hours at ambient temperature, then evaporated down. The residue is extracted with ethyl acetate and water. The organic phase is dried and evaporated to dryness.

Yield: 7.50 g (100% of theoretical)

### N-hydroxy-cyclopropanecarboxamidine

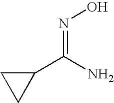

6.00 g (43.41 mmol) potassium carbonate are dissolved in 19 ml of water, 60 ml of ethanol, 3.00 g (43.17 mmol) hydroxylamine and 3.25 ml (43.25 mmol) cyclopropylcyanide are added. The reaction mixture is stirred for 18 hours at ambient temperature, then evaporated down and re-evaporated with toluene. The residue is mixed with ethanol, suction filtered and the filtrate is evaporated to dryness.

Yield: 3.47 g (80% of theoretical)

### tert-butyl [2-(3-cyclopropyl-[1,2,4]oxadiazol-5-yl )-ethyl]-carbamate

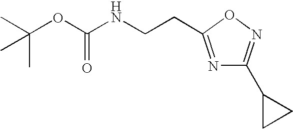

3.10 g (30.96 mmol) N-hydroxy-cyclopropanecarboxamidine are placed in 10 ml dimethylformamide and molecular sieve. 1.32 g (34.06 mmol) sodium hydride (60% in mineral oil) are added. The mixture is stirred for 0.1 hours at 50° C., then 7.40 g (34.06 mmol) ethyl 3-tert-butoxycarbonylamino-propionate in 20 ml dimethylformamide are added. The reaction mixture is stirred for 3 hours at 50° C. After cooling 15 ml of water are added, and the mixture is suction filtered through Celite. The 2 phases of the filtrate are separated, the aqueous phase is extracted with ethyl acetate, the combined organic phase is evaporated down. The residue is purified by chromatography.

Yield: 4.00g (51 % of theoretical)

### 2-(3-cyclopropyl-[1,2,4]oxadiazol-5-yl)-ethylamine hydrochloride (XV.21)

4.00 g (15.79 mmol) tert-butyl [2-(3-cyclopropyl-[1,2,4]oxadiazol-5-yl)-ethyl]-carbamate are placed in 40 ml dichloromethane, 80 ml 1 molar ethereal hydrochloric acid are added. The reaction mixture is stirred for 3 hours at reflux temperature and 72 hours at ambient temperature, then evaporated down. The residue is dissolved in acetone, mixed with diethyl ether and suction filtered.

Yield: 1.30 g (43% of theoretical)

Synthesis of Intermediate Compounds

### Synthesis of Intermediate Compound (VI.1) According to Diagram 1

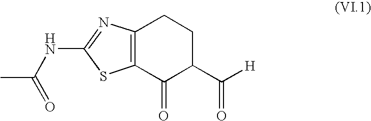

20 g (0.37 mol) sodium methoxide are suspended in 50 ml dimethylformamide, a suspension of 21 g (0.1 mol) intermediate compound (II) in 100 ml dimethylformamide is added dropwise. The mixture is stirred for another 15 minutes, then cooled to 0° C. A mixture of 29.9 ml (0.37 mol) ethyl formate and 60 ml benzene is added dropwise and the reaction mixture is diluted with another 100 ml benzene. Gradually a precipitate settles out and stirring is continued at 0° C. for 3.5 hours. The suspension is hydrolysed with 370 ml 1 molar hydrochloric acid, the solid precipitated is suction filtered. The two phases of the mother liquor are separated, the aqueous phase is extracted with dichloromethane. The resulting organic phase is dried and evaporated to dryness. The solid and the residue from the extraction are recrystallised from acetonitrile.

Yield: 20 g of the intermediate compound (VI.1)

### Synthesis of Intermediate Compound (IX.1) According to Diagram 1

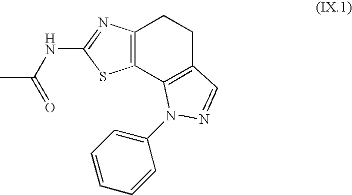

5.00 g (21 mmol) of the intermediate compound (VI.1) are placed in 50 ml glacial acetic acid, 2.13 ml (21 mmol) phenyl-hydrazine are added. The reaction mixture is stirred for 2.5 hours at 60° C., then diluted with 50 ml of water. The precipitate formed is suction filtered and dried. Recrystallisation from acetonitrile.

Yield: 4.39 g (67% of theoretical) of the intermediate compound (IX.1)

mp: 295°-298° C.

### Synthesis of Intermediate Compound (XI.1) According to Diagram 1

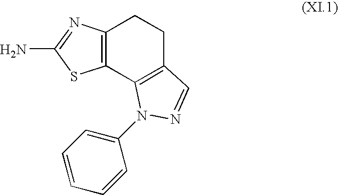

650 ml 37% hydrochloric acid are placed in 650 ml of water and 99 g (0.27 mol) of the intermediate compound (IX.1) are dissolved therein. The solution is refluxed for 2 hours with stirring. After cooling to ambient temperature it is carefully made basic with sodium hydroxide solution (pH 10-11). The precipitate formed is suction filtered and stirred with methanol. Yield: 66 g (m.p.: 307-308° C.) of the intermediate compound (XI.1).

### Synthesis of Intermediate Compound (XII.1) According to Diagram 1

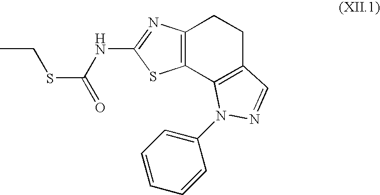

2.7 g (9.9 mmol) of intermediate compound (XI.1) are placed in 70 ml of pyridine and heated to 50° C. To this suspension are added 1.6 ml (15 mmol) ethylthiochloroformate. The resulting solution is stirred for 2 hours at 50° C. After cooling to ambient temperature the solution is added to 700 ml of water, the precipitate is formed suction filtered, washed and dried. Yield: 2.2 g of the intermediate compound (XII.1).

### Synthesis of Intermediate Compound (VlI.1) According to Diagram 1

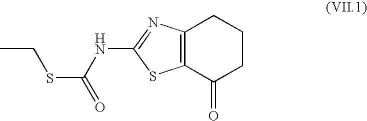

60.73 g (361 mmol) 2-amino-5,6-dihydro-4H-benzothiazol-7-one are placed in 400 ml of tetrahydrofuran, 68.02 ml (397 mmol) diisopropylethylamine and 0.100 g dimethylaminopyridine are added. While cooling with ice the mixture is combined with 46.88 g (361 mmol) ethylchlorothioformate. It is refluxed for 3 hours with stirring, then 0.05 eq diisopropylethylamine are added. After a further 3.5 hours at reflux temperature and 16 hours at ambient temperature a total of 0.15 eq diisopropylethylamine are added. The reaction mixture is added to water, stirred for 16 hours, cooled to 0° C. and suction filtered. The precipitate is stirred with petroleum ether.

Yield: 65.60 g (71% of theoretical) of the intermediate compound (VIl.1)

### Synthesis of Intermediate Compound (X.1) According to Diagram 1

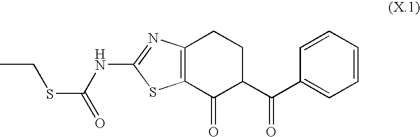

15.00 g (58.51 mmol) intermediate compound (VII.1) are placed in 80 ml of tetrahydrofuran, then cooled to −50° C. Within 0.75 hours 175.50 ml (175.50 mmol) 1 molar solution lithium-bis-(trimethylsilyl)-amid solution in tetrahydrofuran are added dropwise. The mixture is stirred for 1.5 hours at −50° C., then 30.30 g (175.98 mmol) N-methyl-N-methyliminomethyl-benzamide in 100 ml of tetrahydrofuran are slowly added dropwise. The reaction mixture is allowed to come up to ambient temperature within 16 hours. Then it is acidified and added to phosphate buffer. The organic phase is separated off, the aqueous phase is extracted with ethyl acetate. The combined organic phases are dried and evaporated to dryness. The residue is combined with tetrahydrofuran and methyl-tert.butylether. The precipitate formed is suction filtered, the mother liquor is evaporated down.

Yield: 33.00 g of the intermediate compound (X.1)

The intermediate compounds (X.2) to (X.4) can be prepared analogously:

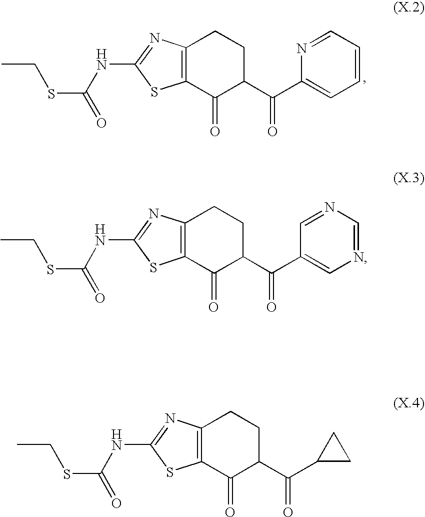

### Synthesis of Intermediate Compound (XII.2) According to Diagram 1

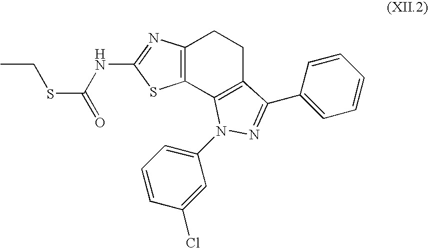

7.00 g (11.65 mmol) of the intermediate compound (X.1) and 3.40 g (18.99 mmol) 3-chlorophenylhydrazine-hydrochloride are stirred in 70 ml glacial acetic acid for 16 hours at ambient temperature and for 3 hours at 50° C. Then the reaction mixture is poured onto water, the precipitate formed is suction filtered, stirred with ethyl acetate and suction filtered again.

Yield: 3.30 g (61% of theoretical) of the intermediate compound (XII.2)

The intermediate compounds (XII.3) to (XII.10) can be prepared analogously from the respective appropriate intermediate compound (X.1) to (X.4) and the respective appropriate hydrazines.

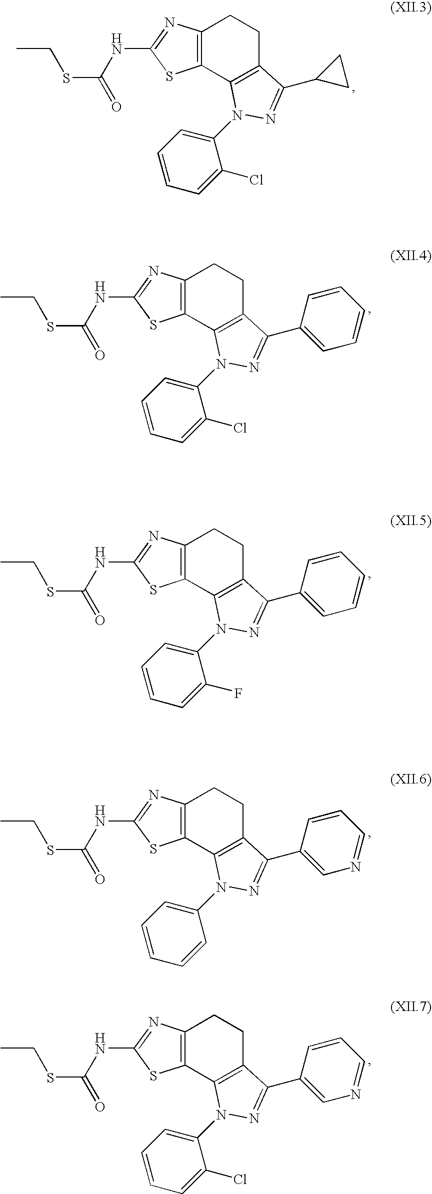

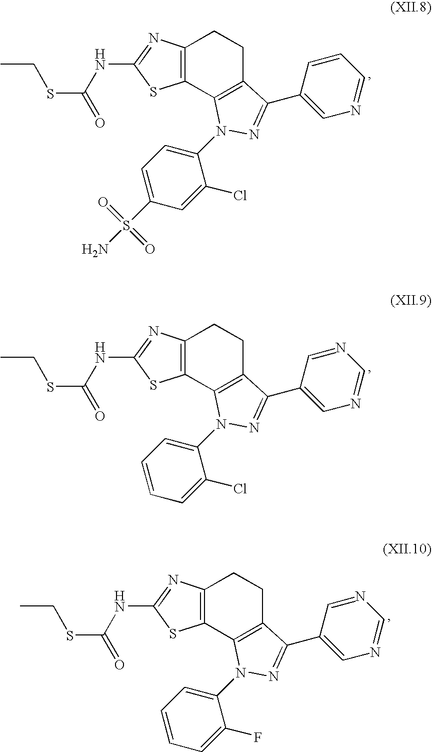

### Synthesis of Intermediate Compound (XII.11) According to Diagram 3

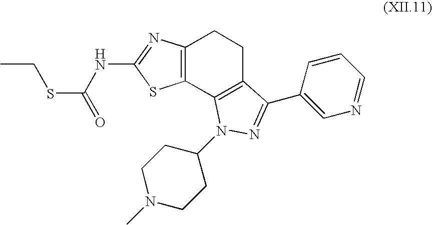

10.73 g (29.69 mmol) of the intermediate compound (X.2) and 6.00 g (29.69 mmol) (1-methyl-piperidin-4-yl)-hydrazine dichloride are stirred in 100 ml glacial acetic acid for 2 weeks at ambient temperature. Then the mixture is evaporated down, the residue is crystallised from acetonitrile.

Yield: 11.25 g (83% of theoretical) of the intermediate compound (XII.11).

The intermediate compounds (XII.12) and (XII.13) can be prepared analogously from the intermediate compound (X.3) and (X.2) by reacting with the respective appropriate hydrazines.

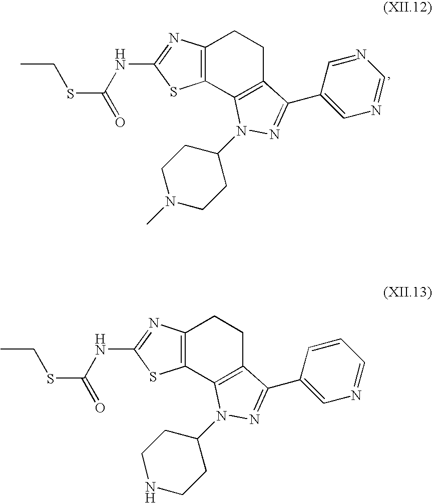

### Synthesis of Intermediate Compound (XII.14) According to Diagram 3

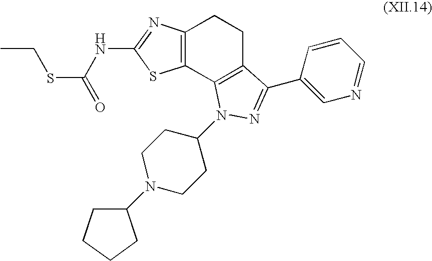

3.00 g (6.81 mmol) of the intermediate compound (XII.13) are dissolved in 150 ml dichloromethane and 150 ml of tetrahydrofuran, and combined with 0.903 ml (10.21 mmol) cyclopentanone. Then 3.50 g (16.51 mmol) sodium triacetoxyborohydride and 0.598 g (7.29 mmol) sodium acetate are added. The reaction mixture is stirred for 48 hours at ambient temperature and 16 hours at 50° C. Then dichloromethane and 5% potassium carbonate solution are added and the mixture is extracted. Any precipitate contained in the aqueous phase is suction filtered and purified by chromatography (RP-HPLC). Corresponding fractions are combined, evaporated down and the base is liberated.

The organic phase is evaporated down, the residue is also purified by chromatography. Corresponding fractions are combined, evaporated down and the base is liberated.

The two substances are combined.

Yield: 1.26 g (36% of theoretical) of the intermediate compound (XII.14)

HPLC-MS: method A, RT=2.25 min, MH+=509

The intermediate compound (XII.15) can be prepared analogously.

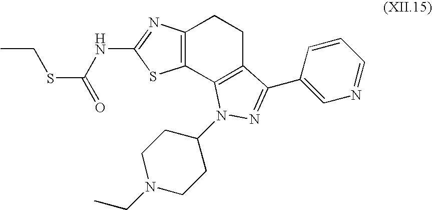

### Synthesis of Intermediate Compound (XII.16) According to Diagram 2

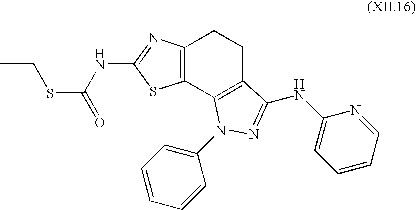

### Intermediate Compound (XVII.1)

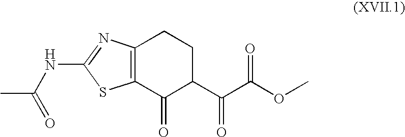

37.98 g (703 mmol) sodium methoxide are suspended in 95 ml dimethylformamide, then a suspension of 40.00 g (190 mmol) N-(7-oxo-4,5,6,7-tetrahydro-benzothiazol-2-yl)-acetamide in 190 ml dimethylformamide and 100 ml benzene is added dropwise within 0.5 hours. It is stirred for 0.25 hours, then cooled to 0° C. 83.86 g (703 mmol) dimethyloxalate in 120 ml benzene and dimethylformamide are added dropwise to the suspension within 0.5 hours. After the addition of 84 ml benzene the reaction mixture is stirred for another 2.5 hours, then hydrolysed with 1 molar hydrochloric acid. The precipitate formed is suction filtered, washed with water and dried, then recrystallised from acetonitrile.

Yield: 51.82 g (92% of theoretical) of the intermediate compound (XVII.1)

### Intermediate Compound (XVIII.1)

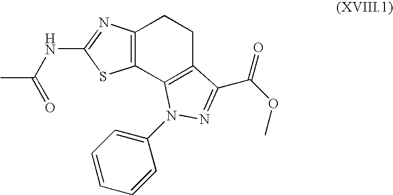

30.00 g (101 mmol) of the intermediate compound (XVII.1) are suspended in 500 ml glacial acetic acid, 10.25 ml (101 mmol) phenylhydrazine are added. The reaction mixture is stirred for 6 hours at 60° C. After cooling water is added, the precipitate is suction filtered, dried and extracted with acetonitrile.

Yield: 26.58 g (71% of theoretical) of the intermediate compound (XVIII.1)

### Intermediate Compound (XIX.1)

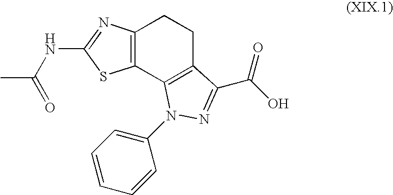

15.50 g (42.07 mmol) of the intermediate compound (XVIII.1) are placed in 180 ml dioxane, 3.00 g (124, 01 mmol) lithium hydroxide in 25 ml of water are added. The reaction mixture is stirred for 16 hours at ambient temperature. 1 eq lithium hydroxide is added, the mixture is stirred for 3 hours at 50° C. and for 16 hours at ambient temperature. The suspension is acidified, then evaporated down. The aqueous residue is diluted with water, suction filtered and dried.

Yield: 16.80 g (100%) of the intermediate compound (XIX.1)

### Intermediate Compound (XX.1)

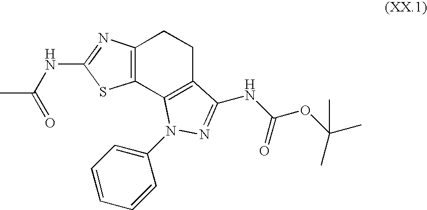

8.50 g (21.75 mmol) of the intermediate compound (XIX.1) are placed in 100 ml of tetrahydrofuran, 7.00 ml (50.09 mmol) triethylamine and 6.00 ml (27.29 mmol) phosphoric acid-diphenylesterazide are added. The reaction mixture is stirred for 72 hours at ambient temperature and for 4 hours at 50° C. 0.5 eq triethylamine and 1 eq phosphoric acid-diphenylesterazide are added, the mixture is stirred for 16 hours at 40° C. and for 24 hours at ambient temperature. Then the precipitate is filtered off and dried.

Yield: 9.00 g

9.00 g (21.35 mmol) of the compound obtained above are suspended in 120 ml tert. butanol and 10 ml trifluoroacetic acid, then heated to 120° C. The mixture is refluxed for 10 hours with stirring, then evaporated to dryness. The residue is again combined with 120 ml tert. butanol and refluxed for 72 hours with stirring. It is evaporated to dryness.

Yield: 12.00 g (92% of theoretical) of the intermediate compound (XX.1)

HPLC-MS: method A, RT=2.89 min MH+=370 and 426 (Boc)

### Intermediate Compound (XXI.1)

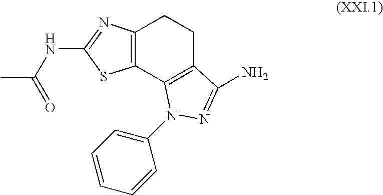

12.00 g (19.74 mmol) of the intermediate compound (XX.1) are placed in 200 ml dichloromethane, 17.50 ml (227.15 mmol) trifluoroacetic acid are added. The mixture is stirred for 24 hours at ambient temperature, then evaporated down. The residue is made basic with sodium hydrogen carbonate solution, dichloromethane is added and the mixture is extracted. The organic phase is dried and evaporated to dryness. The product is crystallised with methanol, methyl-tert.butylether and n-heptane.

Yield: 3.00 g (44% of theoretical) of the intermediate compound (XXI.1)

HPLC-MS: method A, RT=2.18 min, MH+=325

### Intermediate Compound (IX.2)

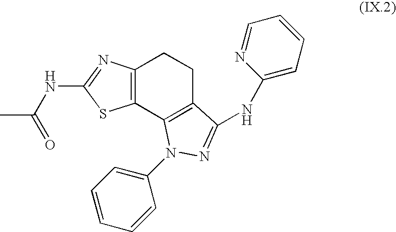

5.00 g (13.83 mmol) of the intermediate compound (XXI.1) and 1.70 ml (17.47 mmol) 2-bromopyridine are dissolved in 20 ml dimethylformamide under an argon atmosphere, 4.10 g (41.81 mmol) sodium tert.butoxide, 0.300 g (1.01 mmol) tri-tert.butylphosphine-tetrafluoroborate and 0.800 g (0.874 mmol) tris(dibenzylideneacetone)-dipalladium (0) are added. The reaction mixture is stirred for 16 hours at 50° C., then filtered through kieselguhr/magnesium sulphate and evaporated down. The residue is purified by chromatography.

Yield: 1.05 g (19% of theoretical) of the intermediate compound (IX.2)

HPLC-MS: method A, RT=2.50 min, MH+=402

### Intermediate Compound (XI.2)

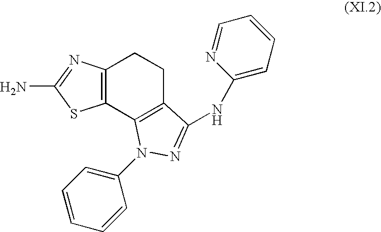

1.00 g (2.49 mmol) of the intermediate compound (IX.2) are dissolved in 20 ml semiconc. hydrochloric acid, then stirred for 5 hours at 80° C. and for 16 hours at ambient temperature. The reaction mixture is made alkaline with sodium hydroxide solution, the precipitate formed is suction filtered and dried.

Yield: 0.600 g (67% of theoretical) of the intermediate compound (XI.2)

HPLC-MS: method A, RT=3.14 min, MH+=360

### Intermediate Compound (XII.16)

600 mg (1.67 mmol) of the intermediate compound (XI.2) are suspended in 20 ml of pyridine and heated to 50° C. 360 μl (3.32 mmol) ethylchlorothiolformate are added dropwise. The mixture is stirred for 3 hours at 55° C. and for 16 hours at ambient temperature. Then the reaction mixture is added dropwise to water, the precipitate formed is suction filtered and dried.

Yield: 550 mg (66% of theoretical) of the intermediate compound (XII.16)

HPLC-MS: method A, RT=1.86 min, MH+=448

### Synthesis of Intermediate Compound (XXV.1)=Example 2 According to Diagram 4

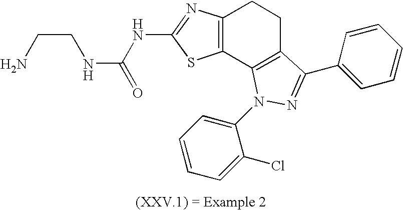

### Intermediate Compound (XXIV.1)=Example 151

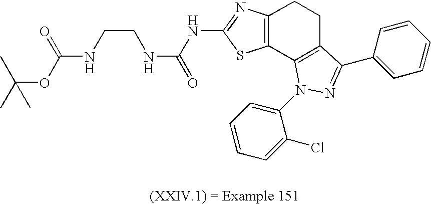

1.20 g (2.06 mmol) of the intermediate compound (XII.4), 0.50 ml (3.12 mmol) N-boc ethylenediamine and 20 μl triethylamine are stirred in 5 ml dioxane for 4 hours at 100° C. After cooling the reaction mixture is extracted with potassium hydrogen carbonate solution, the organic phase is purified by chromatography.

Corresponding fractions are evaporated down, crystallised with ethyl acetate and n-heptane.

Yield: 0.926 g (80% of theoretical) of the intermediate compound (XXIV.1)=Example 151

HPLC-MS: method A, RT=3.49 min, MH+=565/7

### Intermediate Compound (XXV.1)=Example 2

1.20 g (2.12 mmol) of the intermediate compound (XXIV.1) and 7.00 ml (28 mmol) 4 molar hydrochloric acid in dioxane are stirred in 5 ml dioxane for 72 hours at ambient temperature. The precipitate formed is suction filtered, washed with n-heptane and dried.

Yield: 1.07 g (100% of theoretical) of the intermediate compound (XXV.1)=Example 2

HPLC-MS: method A, RT=2.65 min, MH+=465/7

### Synthesis of Intermediate Compound (XXIX.1)=Example 5 According to Diagram 5

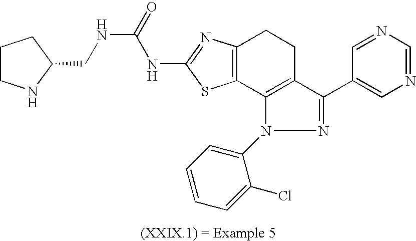

### Intermediate Compound (XXVIII.1)=Example 190

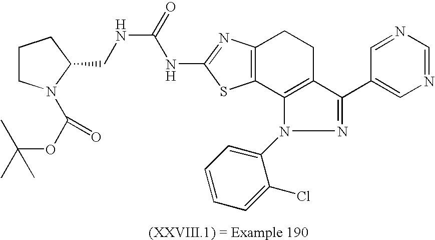

1.00 g (2.13 mmol) intermediate compound (XII.9) and 0.641 g (3 mmol) tert-butyl (R)-2-aminomethyl-pyrrolidine-1-carboxylate (XXVII.1) are stirred in 5 ml of ethanol for 48 hours at 80° C. Then the reaction mixture is purified by chromatography, corresponding fractions are combined and evaporated to dryness. The residue is crystallised from ethyl acetate and petroleum ether.

Yield: 1.23 g (81% of theoretical) of the intermediate compound (XXVIII.1)=Example 190

MP: 155° C.

### Intermediate Compound (XXIX.1)=Example 5

25 mg (0.035 mmol) of the intermediate compound (XXVIII.1) are stirred in 5 ml of 4 molar hydrochloric acid in dioxane for 1 hour at ambient temperature. The reaction mixture is evaporated down, the residue is combined with ethyl acetate/methanol and methyl-tert.butylether. The precipitate formed is suction filtered and evaporated to dryness.

Yield: 19 mg (100% of theoretical) of the intermediate compound (XXIX.1)=Example 5.

MP: <100° C.

The intermediate compounds (XXVIII.2)=Example 188 and (XXIX.2)=Example 3 can be prepared analogously by using the compound tert-butyl (S)-2-aminomethyl-pyrrolidine-1-carboxylate (XXVII.2) which is enantiomeric to (XXVII.1).

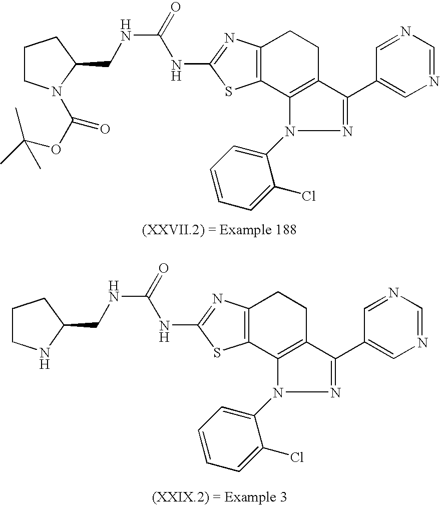

The intermediate compounds (XXVIII.3)=Example 191 and (XXIX.3)=Example 20 can be prepared analogously by using tert-butyl (R)-3-aminomethyl-pyrrolidine-1-carboxylate (XXVI1.3).

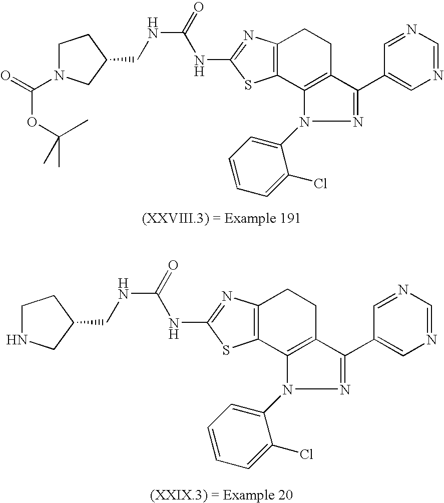

The intermediate compounds (XXVIII.4)=Example 189 and (XXIX.4)=Example 4 can be prepared analogously using tert-butyl (S)-3-aminomethyl-pyrrolidin-1-carboxylate (XXVII.4).

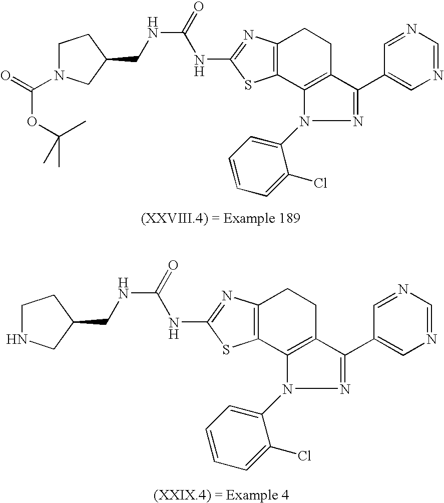

Moreover the following intermediate compounds may be prepared analogously by reacting the respective appropriate intermediates (XII) with the respective appropriate tert-butylaminomethyl-pyrrolidin-1-carboxylates and subsequent deprotection:

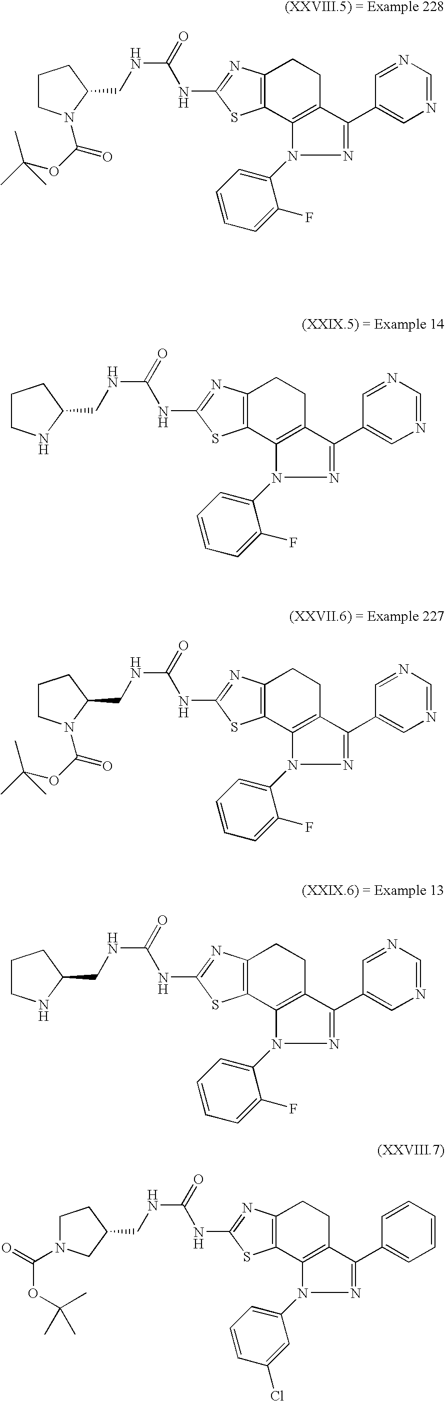

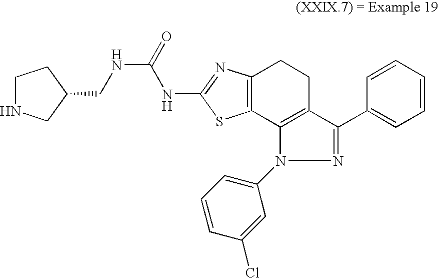

### Synthesis of Intermediate Compound (XXXVI.1) According to Diagram 6

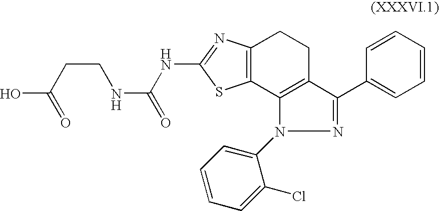

### Intermediate Compound (XXXV.1)

500 mg (1.07 mmol), of the intermediate compound (XII.4) 270 mg (1.61 mmol) ethyl-4-aminobutyrate hydrochloride and 450 μl (3.25 mmol) triethylamine are stirred in 25 ml of ethanol for 16 hours at 80° C. The reaction mixture is evaporated down, the residue is purified by chromatography. Corresponding fractions are evaporated down and extracted with acetonitrile.

Yield: 470 mg (82% of theoretical) of the intermediate compound (XXXV.1)

### Intermediate Compound (XXXVI.1)

430 mg (0.802 mmol) of the intermediate compound (XXXV.1) and 3.00 ml (1.50 mmol) 0.5molar sodium hydroxide solution are stirred in 1.20 ml of methanol for 48 hours at ambient temperature. A further 0.3 eq sodium hydroxide solution are added, the mixture is stirred for 5 hours at 50° C. and for 72 hours at ambient temperature. Then the solution is acidified and evaporated down. The precipitate is suction filtered, washed and dried.

Yield: 370 mg (68% of theoretical) of the intermediate compound (XXXVI.1)

HPLC-MS: method A, RT=3.09 min, MH+=508/510

### Synthesis of Intermediate Compound (XXXX.1) According to Diagram 7

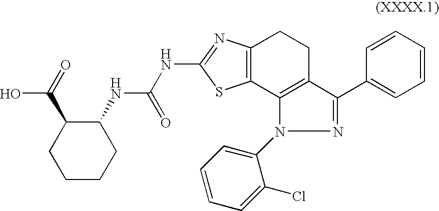

### Intermediate Compound (XXXIX.1)

500 mg (1.07 mmol) of the intermediate compound (XII.4), 334 mg (1.61 mmol) ethyl-trans-2-amino-1-cyclohexancarboxylat hydrochloride and 450 μl (3.25 mmol) triethylamine are stirred in 25 ml of ethanol 16 hours at 80° C. The reaction mixture is evaporated down, the residue is purified by chromatography. Corresponding fractions are evaporated down and extracted with acetonitrile.

Yield: 470 mg (76% of theoretical) of the intermediate compound (XXXIX.1)

### Intermediate Compound (XXXX.1)

430 mg (0.746 mmol) of the intermediate compound (XXXIX.1) and 3.00 ml (1.50 mmol) 0.5molar sodium hydroxide solution are stirred in 1.20 ml of methanol for 48 hours at ambient temperature. The reaction mixture is made neutral and evaporated down. The residue is stirred with 5 ml dioxane and 100 mg lithium hydroxide for 16 hours at ambient temperature, then acidified. The precipitate formed is suction filtered, washed with water and dried.

Yield: 360 mg (60% of theoretical) of the intermediate compound (XXXX.1)

HPLC-MS: method A, RT=3.34 min,

Synthesis of the Compounds of Formula (I)

The following HPLC-MS methods were used to characterise the compounds of formula (I):

Methods A and B:

Waters ZMD, Alliance 2690/2695 HPLC, Waters 2700 Autosampler, Waters 996/2996 Diode array detector (wavelength range 210-400 nm).

Stationary phase (column temperature: constant at 25° C.):

Method A: column XTerra®, MS C₁₈ 2.5 μm, 4.6 mm×30 mm.

Method B: column Merck Chromolith™ SpeedROD RP-18e, 4.6 mm×50 mm.

Mobile phase: L1: water with 0.10% TFA; L2: acetonitrile with 0.10% TFA flow rates:

Method A: 1.00 mLl/min

Method B: 2.00 mL/min

| time (min) | % L1 | % L2 |
|---|---|---|
| 0.0 | 95 | 5 |
| 0.1 | 95 | 5 |
| 3.1 | 2 | 98 |
| 4.5 | 2 | 98 |
| 5.0 | 95 | 5 |

Methods C and D:

Waters ZMD, Alliance 2790/2795 HPLC, Waters 2700 Autosampler, Waters 996/2996 Diode array detector (wavelength range 210-500 nm). Stationary phase (column temperature: constant at 40° C.):

column X-Terra MS C18 4.6×50 mm, 3.5 μm.

Mobile phase: L1: water with 0.10% TFA; L2: acetonitrile with 0.10% TFA flow rates: 1.00 mL/min

| time (min) | % L1 | % L2 |
|---|---|---|
| 0.0 | 95 | 5 |
| 0.1 | 95 | 5 |
| 5.1 | 2 | 98 |
| 6.5 | 2 | 98 |
| 7.0 | 95 | 5 |

The symbol X used in Table A in the structural formula of the substituent is to be understood as being the linkage point to the remainder of the molecule. The substituent replaces the groups R^{a}, R^{b} and R^{c} according to the arrangement of the columns.

### EXAMPLES

### Synthesis of Example 94

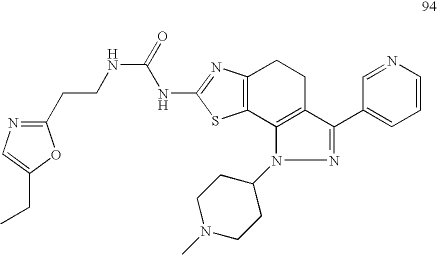

100 mg (0.220 mmol) of the intermediate (XII.11), 41 mg (0.232 mmol) 2-(5-ethyl-oxazol-2-yl)-ethylamine and 300 ml triethylamine are stirred in 10 ml of ethanol for 24 hours at 80° C. The reaction mixture is evaporated down, the residue is purified by chromatography (HPLC).

Yield: 100 mg (85% of theoretical)

Examples 16-18, 21-93, 95-150, 152-187, 192-226, 229-239, 344-347 and 308 may be prepared analogously using the respective appropriate intermediates (XII) and the respective amines.

### Synthesis of Example 248

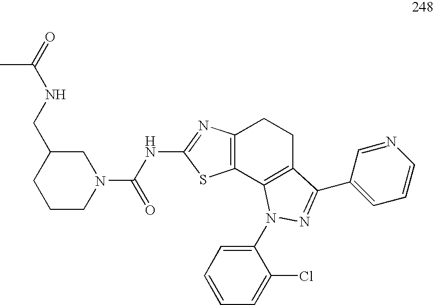

21 mg (0.045 mmol) of the intermediate compound (XII.1) and 20 mg (0.2 mmol) triethylamine are placed in 1 ml of ethanol, 10 mg (0.067 mmol) N-piperidin-3-ylmethyl-acetamide in 1 ml of ethanol are added. The reaction mixture is stirred for 16 hours at 70° C. Then the mixture is evaporated down, the residue is purified by chromatography (LCMS). Corresponding fractions are lyophilised.

Yield: 18 mg (72% of theoretical)

Examples 240-247 and 249-307 may be prepared analogously by reacting the intermediate compound (XII.1) with the corresponding amines.

### Synthesis of Example 317

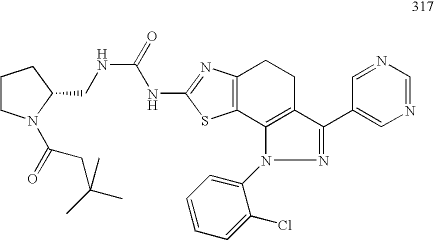

80 mg (0.125 mmol) of the intermediate (XXIX.1) and 104 μl (0.603 mmol) diisopropylethylamine are placed in 4 ml of tetrahydrofuran, 20 mg (0.150 mmol) 3,3-dimethyl-butyryl chloride are added while cooling with ice. The mixture is stirred for 20 hours at ambient temperature, then water and tetrahydrofuran are added and the mixture is extracted. The organic phase is purified by chromatography. Corresponding fractions are combined and evaporated down. The residue is crystallised with ethyl acetate and petroleum ether.

Yield: 34 mg (45% of theoretical)

mp: 208° C.

Examples 1, 9, 318-319 and 323-339 may be prepared analogously by reacting the respective appropriate intermediates (XXIX) with the respective appropriate acylating reagents (XXX)-(XXXIII) according to Diagram 5.

### Synthesis of Example 321

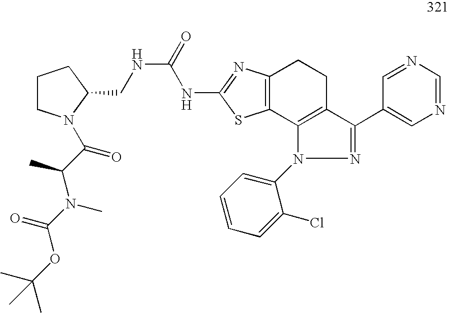

150 mg (0.276 mmol) of the intermediate compound (XXIX.1), 62 mg (0.304 mmol) (S)-2-(tert-butoxycarbonyl-methyl-amino)-propionic acid, 0.130 ml (0.759 mmol) diisopropylethylamine and 115 mg (0.304 mmol) O-(7-azabenzotriazol-1-yl-)-N,N,N′,N′-tetramethyluronium-hexafluoro-phosphate (HATU) are stirred in 2 ml N-methyl-2-pyrrolidone for 1.5 hours at ambient temperature. Then water and dichloromethane are added and the mixture is extracted. The organic phase is dried and evaporated to dryness. The residue is purified by chromatography, the still contaminated product is purified by HPLC. Corresponding fractions are combined and lyophilised.

Yield: 76 mg (40% of theoretical)

HPLC-MS: method A, RT=3.15 min

Examples 320 and 322 may be prepared analogously by reacting the intermediate compound (XXIX.3) or (XXIX.1) with the respective amino acid derivatives.

### Synthesis of Example 313

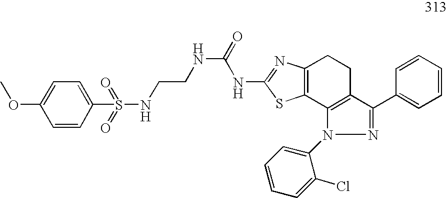

90 mg (0.179 mmol) of the intermediate compound (XXV.1), 49 mg (0.237 mmol) 4-methoxysulphonic acid chloride and 75 μl (0.450 mmol) diisopropylethylamine are stirred in 2 ml dichloromethane for 2 hours at ambient temperature. Then the reaction mixture is extracted with dichloromethane and water, the organic phase is dried and evaporated to dryness. The residue is purified by chromatography, corresponding fractions are evaporated down. The residue is extracted with ethyl acetate.

Yield: 46 mg (36% of theoretical)

HPLC-MS: method B, RT=2.21 min, MH+=635/637

Examples 309-312 and 314-316 may be prepared analogously by reacting the intermediate compound (XXV.1) with the respective appropriate acylating reagents.

### Synthesis of Example 342

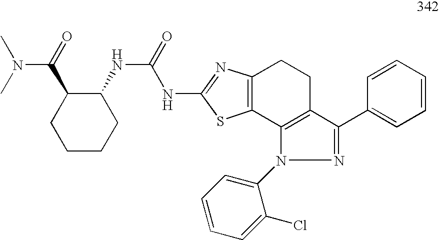

90 mg (0.164 mmol) of the intermediate compound (XXXX.1) , 75 mg (0.197 mmol) O-(7-azabenzotriazol-1-yl-)-N,N,N′,N′-tetramethyluronium-hexafluoro-phosphate (HATU) and 150 μl (0.882 mmol) diisopropylethylamine are placed in 2 ml dichloromethane, then stirred for 0.3 hours at ambient temperature. 20 μl (0.400 mmol) dimethylamine are added, the mixture is stirred for 16 hours at ambient temperature. The reaction mixture is diluted with dichloromethane, extracted with dilute potassium hydrogen carbonate solution and water. The organic phase is dried and evaporated to dryness. The residue is purified by chromatography (semiprep. HPLC). Corresponding fractions are lyophilised.

Yield: 30 mg (32% of theoretical)

HPLC-MS: method A, RT=3.36 min, MH+=575/577

Examples 340, 341 and 343 may be prepared analogously by reacting the intermediate compounds (XXXVI.1) or (XXXX.1) with the appropriate amines

### Synthesis of Example 6

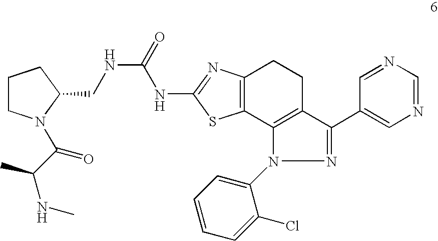

34 mg (0.049 mmol) of the Example compound 321 are stirred in 10 ml of 4 molar hydrochloric acid in dioxane for 2 hours at ambient temperature. Then it is purified by chromatography (prep. HPLC). Corresponding fractions are lyophilised.

Yield: 22 mg (63% of theoretical)

HPLC-MS: method A, RT=2.40 min

Examples 8, 10, 11, 12 and 15 may be prepared analogously by deprotecting the Example compounds 320, 193, 208, 209 and 25.

### Synthesis of Example 7

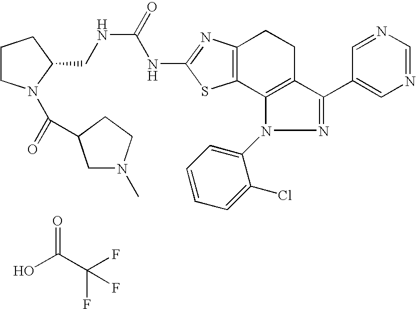

The Example compound 322 is converted into the corresponding free amine analogously to Example 6. The product obtained is then used in the next step.

10 mg (0.333 mmol) paraformaldehyde are placed in 2 ml of tetrahydrofuran, 0.20 ml glacial acetic acid, 100 mg (0.111 mmol) of the amine described above and 189 mg (0.889 mmol) sodium triacetoxyborohydride are added. The reaction mixture is stirred for 16 hours at ambient temperature. After the addition of potassium carbonate solution and dichloromethane the mixture is extracted. The organic phase is dried and evaporated to dryness. The residue is purified by chromatography. Corresponding fractions are lyophilised.

Yield: 16 mg (16% of theoretical)

HPLC-MS: method A, RT=1.58 min, MH+=618

The following compounds are prepared analogously:

TABLE A

| | | | | | | (IA) |
|---|---|---|---|---|---|---|
| 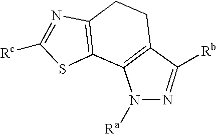 | | | | | | |
| Ex- am- ple no. | 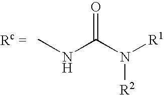 | R^{a} | R^{b} | mp [° C.] | HPLC method | RT [min] |
| 1 | 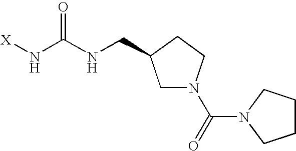 | 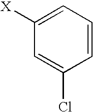 | 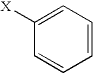 | 252.5 | | |
| 2 | 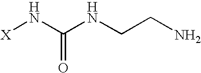 | 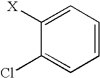 | 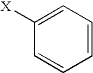 | | A | 2.65 |
| 3 | 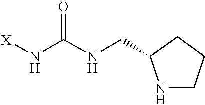 | 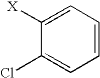 | 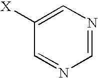 | | | |
| 4 | 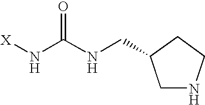 | 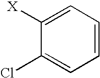 | 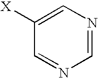 | | A | 2.30 |
| 5 | 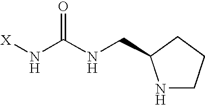 | 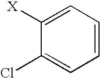 | 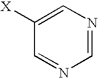 | | | |
| 6 | 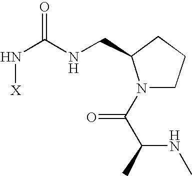 | 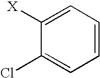 | 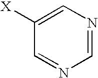 | | A | 2.40 |
| 7 | 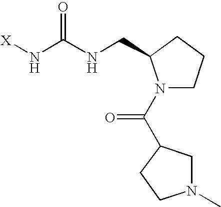 | 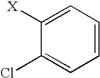 | 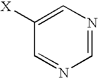 | | B | 1.58 |
| 8 | 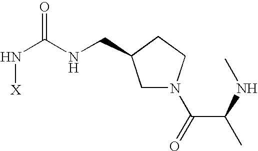 | 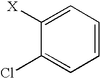 | 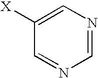 | 122.5 | | |
| 9 | 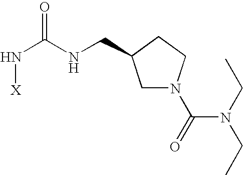 | 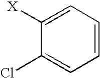 | 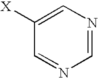 | 129.2 | | |
| 10 | 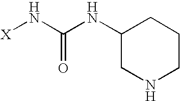 | 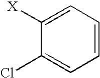 | 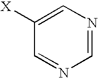 | | A | 2.28 |
| 11 | 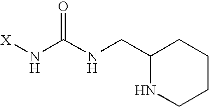 | 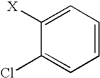 | 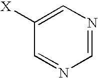 | | | |
| 12 | 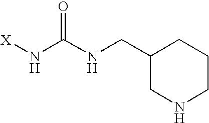 | 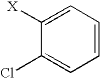 | 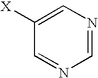 | | A | 2.30 |
| 13 | 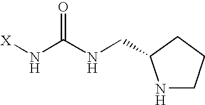 | 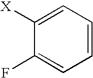 | 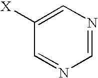 | 97.8 | | |
| 14 | 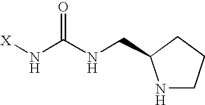 | 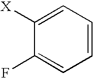 | 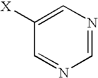 | 243.0 | | |
| 15 | 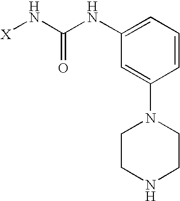 | 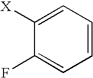 | 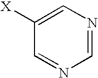 | 97.3 | | |
| 16 | 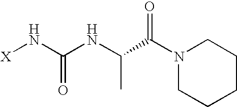 | 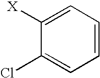 | 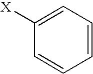 | | A | 3.48 |
| 17 | 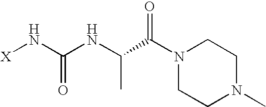 | 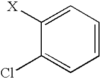 | 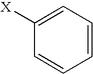 | | A | 2.74 |
| 18 | 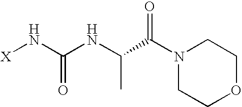 | 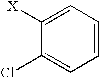 | 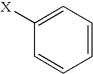 | | A | 3.76 |
| 19 | 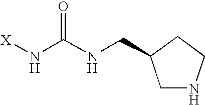 | 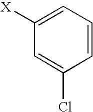 | 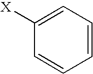 | 279.3 | | |
| 20 | 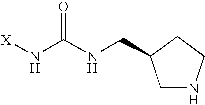 | 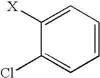 | 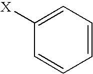 | | A | 2.99 |
| 22 | 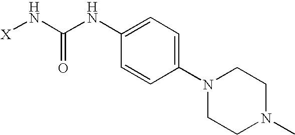 | 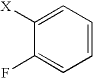 | 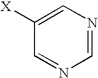 | 97.1 | | |
| 23 | 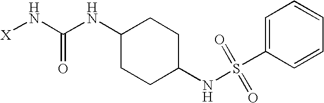 | 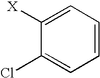 | 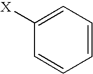 | | | |
| 24 | 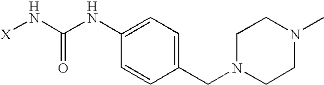 | 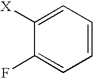 | 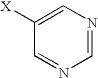 | 204.1 | | |
| 25 | 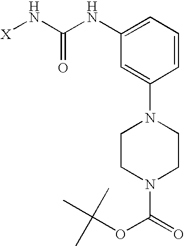 | 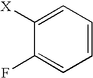 | 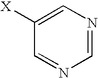 | 231.1 | | |
| 26 | 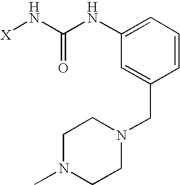 | 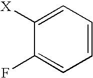 | 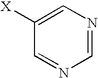 | 266.4 | | |
| 27 | 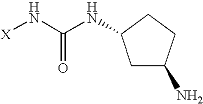 | 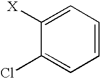 | 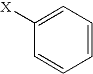 | 177.4 | | |
| 28 | 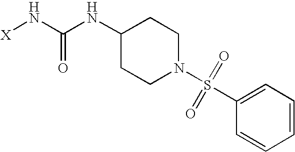 | 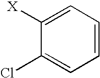 | 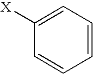 | 287.4 | | |
| 29 | 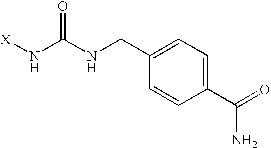 | 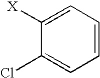 | 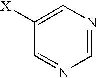 | | B | 1.74 |
| 30 | 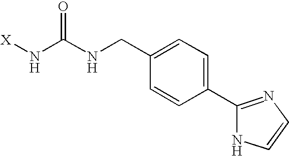 | 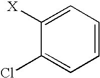 | 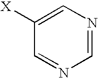 | | A | 2.49 |
| 31 | 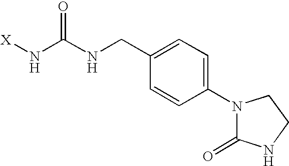 | 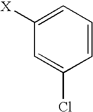 | 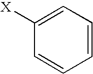 | 296.1 | | |
| 32 | 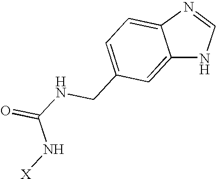 | 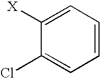 | 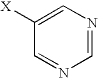 | | A | 2.44 |
| 33 | 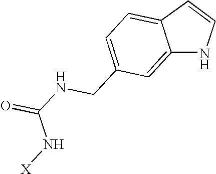 | 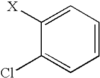 | 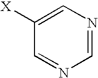 | | A | 3.08 |
| 34 | 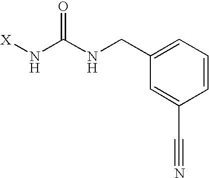 | 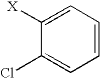 | 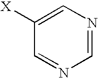 | | A | 3.02 |
| 35 | 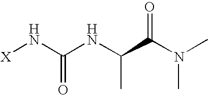 | 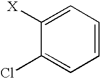 | 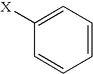 | | A | 3.16 |
| 36 | 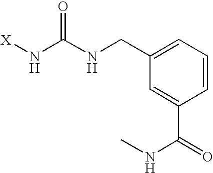 | 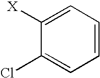 | 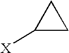 | | | |
| 37 | 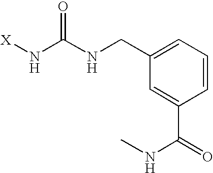 | 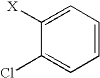 | 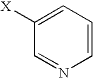 | | A | 1.61 |
| 38 | 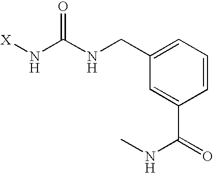 | 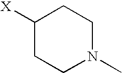 | 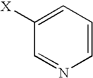 | | A | 1.36 |
| 39 | 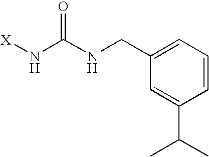 | 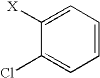 | 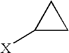 | | A | 3.59 |
| 40 | 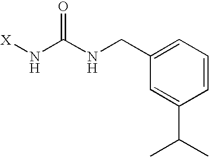 | 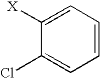 | 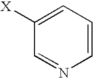 | | A | 2.91 |
| 41 | 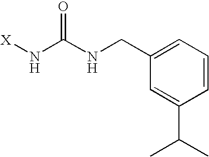 | 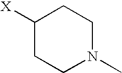 | 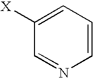 | | A | 2.39 |
| 42 | 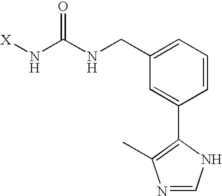 | 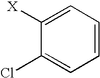 | 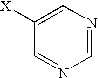 | | A | 2.47 |
| 43 | 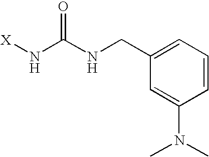 | 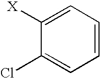 | 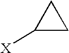 | | | |
| 44 | 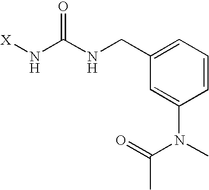 | 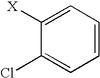 | 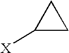 | | | |
| 45 | 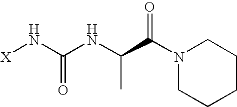 | 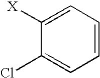 | 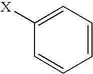 | | | |
| 46 | 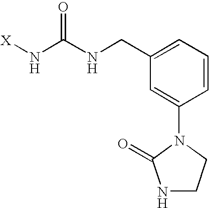 | 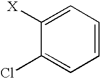 | 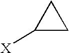 | | | |
| 47 | 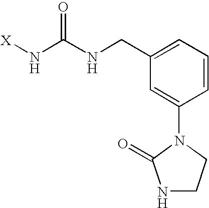 | 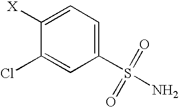 | 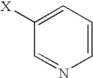 | | A | 2.26 |
| 48 | 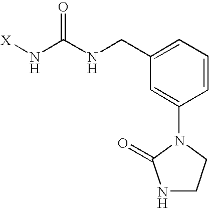 | 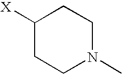 | 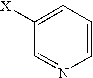 | | A | 2.06 |
| 49 | 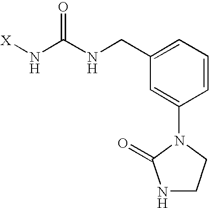 | 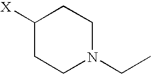 | 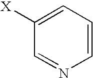 | | A | 2.06 |
| 50 | 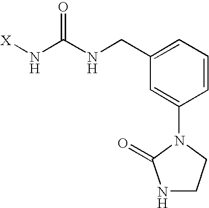 | 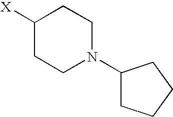 | 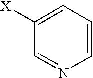 | | A | 2.15 |
| 51 | 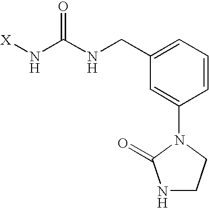 | 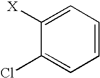 | 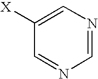 | 237.1 | | |
| 52 | 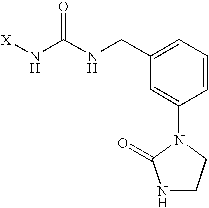 | 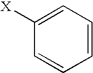 | 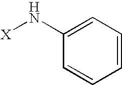 | | A | 2.58 |
| 53 | 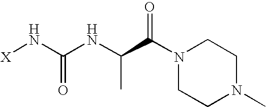 | 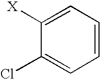 | 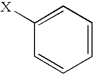 | | A | 2.73 |
| 54 | 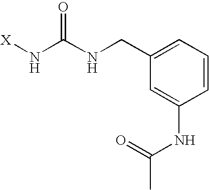 | 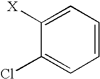 | 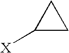 | | | |
| 55 | 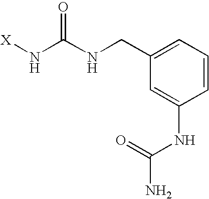 | 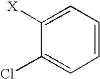 | 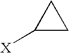 | | A | 1.88 |
| 56 | 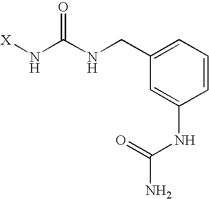 | 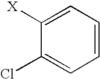 | 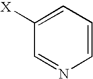 | | A | 1.60 |
| 57 | 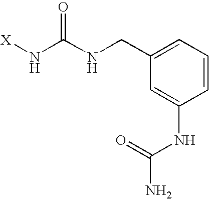 | 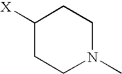 | 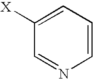 | | A | 1.33 |
| 58 | 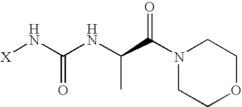 | 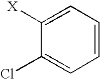 | 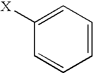 | | | |
| 59 | 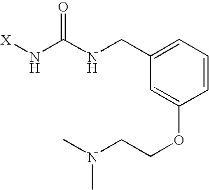 | 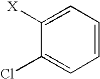 | 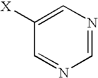 | | B | 1.74 |
| 60 | 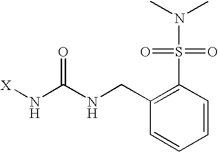 | 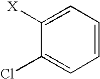 | 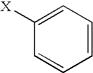 | 154.8 | | |
| 61 | 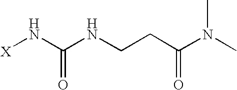 | 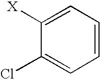 | 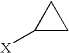 | | A | 2.83 |
| 62 | 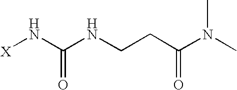 | 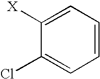 | 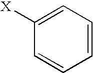 | 166.5 | | |
| 63 | 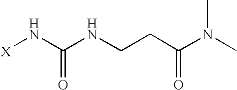 | 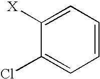 | 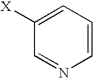 | | A | 2.88 |
| 64 | 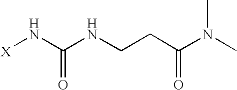 | 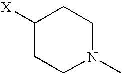 | 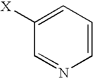 | | A | 1.92 |
| 65 | 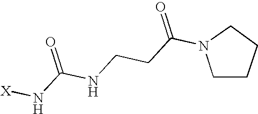 | 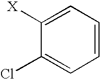 | 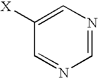 | | A | 2.63 |
| 66 | 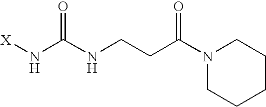 | 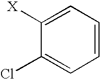 | 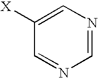 | | A | 2.79 |
| 67 | 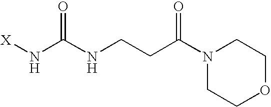 | 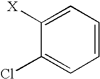 | 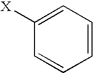 | 139.2 | | |
| 68 | 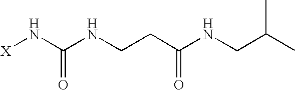 | 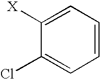 | 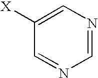 | | A | 2.71 |
| 69 | 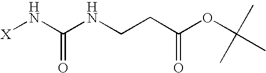 | 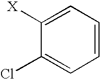 | 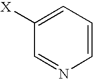 | | | |
| 70 | 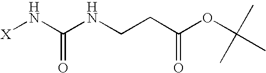 | 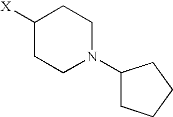 | 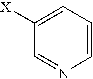 | | A | 2.38 |
| 71 | 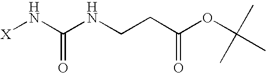 | 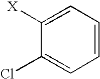 | 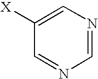 | 203.3 | | |
| 72 | 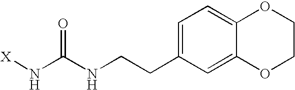 | 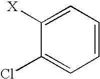 | 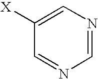 | | | |
| 73 | 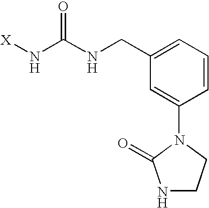 | 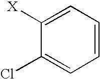 | 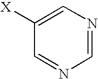 | | | |
| 74 | 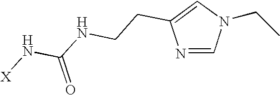 | 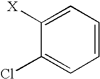 | 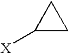 | | | |
| 75 | 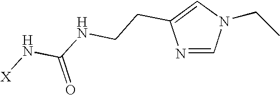 | 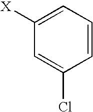 | 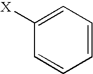 | 204.1 | | |
| 76 | 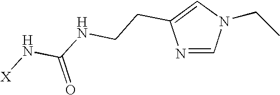 | 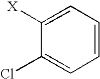 | 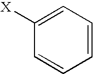 | 128.7 | | |
| 77 | 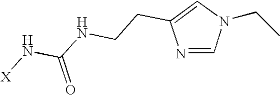 | 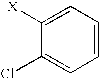 | 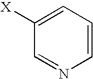 | | A | 2.07 |
| 78 | 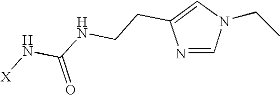 | 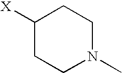 | 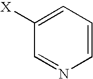 | | | |
| 79 | 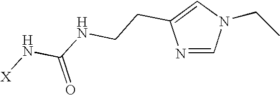 | 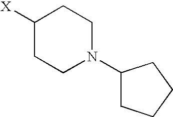 | 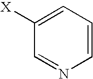 | | A | 1.90 |
| 80 | 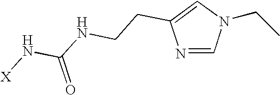 | 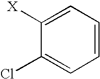 | 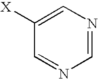 | 253.6 | | |
| 81 | 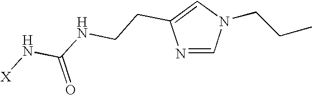 | 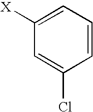 | 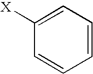 | 253.1 | | |
| 82 | 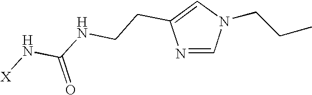 | 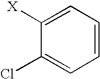 | 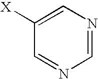 | 253.4 | | |
| 83 | 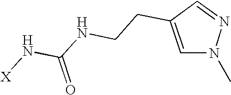 | 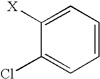 | 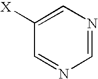 | | | |
| 84 | 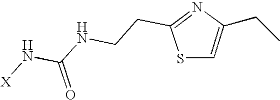 | 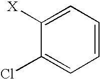 | 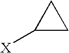 | | | |
| 85 | 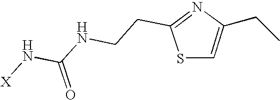 | 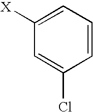 | 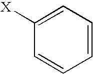 | | | |
| 86 | 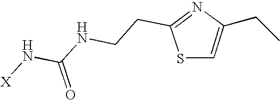 | 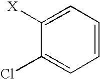 | 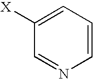 | | | |
| 87 | 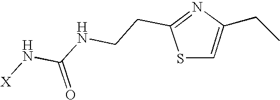 | 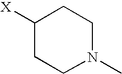 | 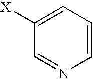 | | | |
| 88 | 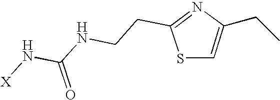 | 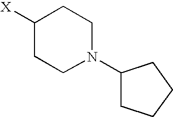 | 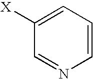 | | A | 2.07 |
| 89 | 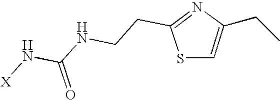 | 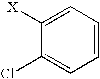 | 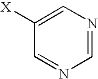 | 174.8 | | |
| 90 | 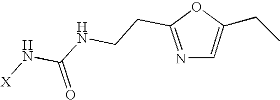 | 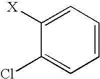 | 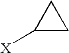 | | | |
| 91 | 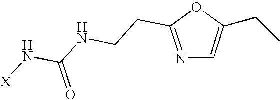 | 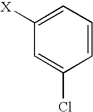 | 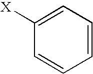 | 256.5 | | |
| 92 | 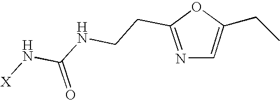 | 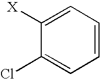 | 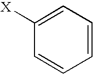 | | | |
| 93 | 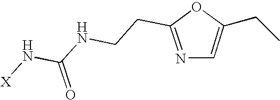 | 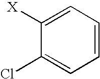 | 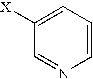 | | | |
| 94 | 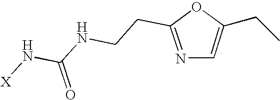 | 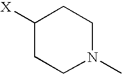 | 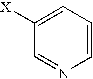 | | | |
| 95 | 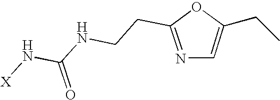 | 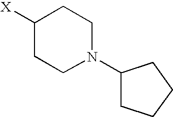 | 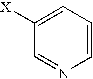 | | A | 2.17 |
| 96 | 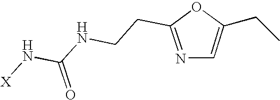 | 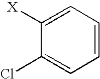 | 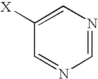 | 196.5 | | |
| 97 | 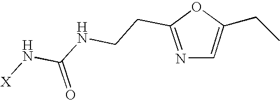 | 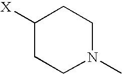 | 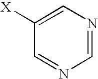 | | A | 2.16 |
| 98 | 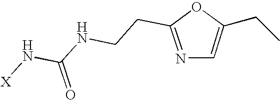 | 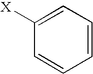 | 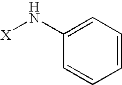 | | A | 2.60 |
| 99 | 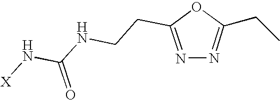 | 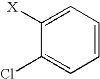 | 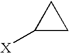 | | | |
| 100 | 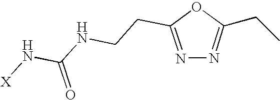 | 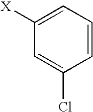 | 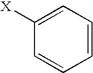 | | | |
| 101 | 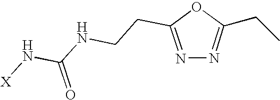 | 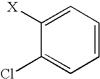 | 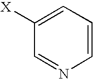 | | | |
| 102 | 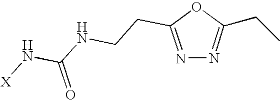 | 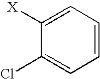 | 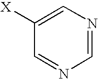 | <100 | | |
| 103 | 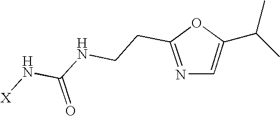 | 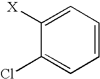 | 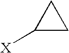 | | | |
| 104 | 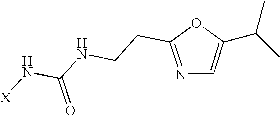 | 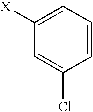 | 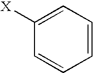 | 262.2 | | |
| 105 | 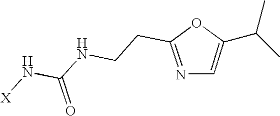 | 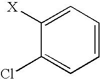 | 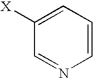 | | | |
| 106 | 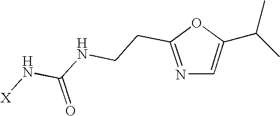 | 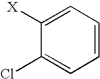 | 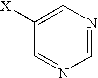 | | | |
| 107 | 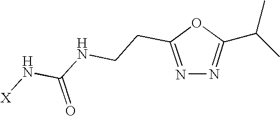 | 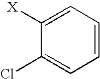 | 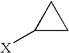 | | | |
| 108 | 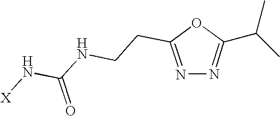 | 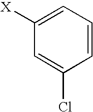 | 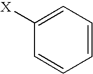 | 218.1 | | |
| 109 | 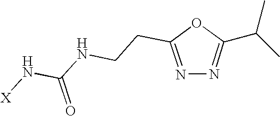 | 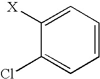 | 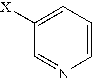 | | | |
| 110 | 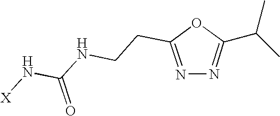 | 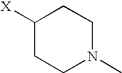 | 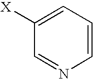 | | | |
| 111 | 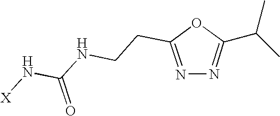 | 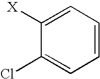 | 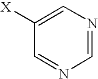 | | | |
| 112 | 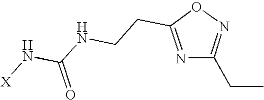 | 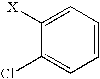 | 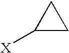 | | | |
| 113 | 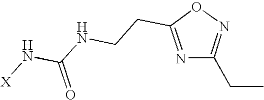 | 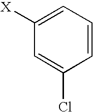 | 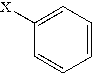 | 272.7 | | |
| 114 | 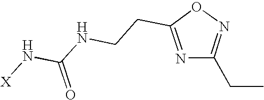 | 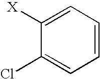 | 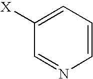 | | | |
| 115 | 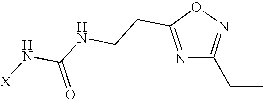 | 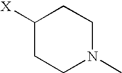 | 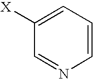 | | | |
| 116 | 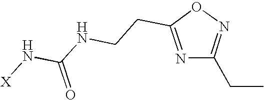 | 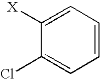 | 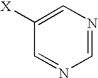 | 141.6 | | |
| 117 | 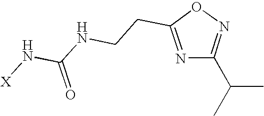 | 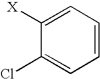 | 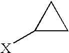 | | | |
| 118 | 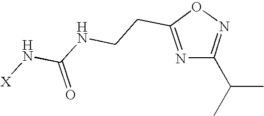 | 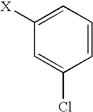 | 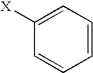 | 255.3 | | |
| 119 | 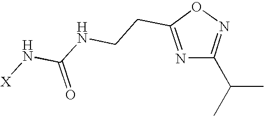 | 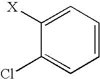 | 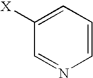 | | | |
| 120 | 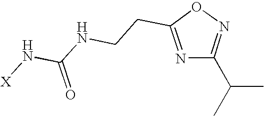 | 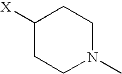 | 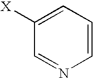 | | | |
| 121 | 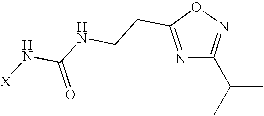 | 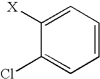 | 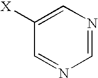 | 200.6 | | |
| 122 | 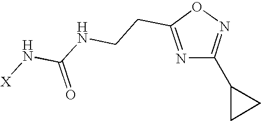 | 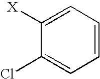 | 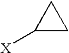 | | | |
| 123 | 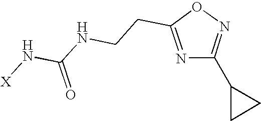 | 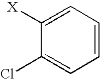 | 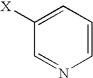 | | | |
| 124 | 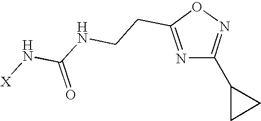 | 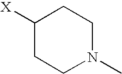 | 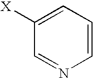 | | | |
| 125 | 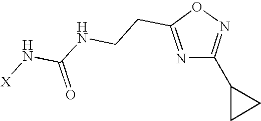 | 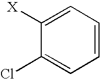 | 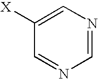 | 224.3 | | |
| 126 | 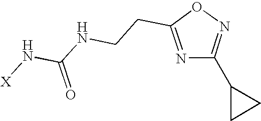 | 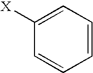 | 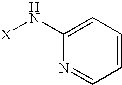 | | A | 2.67 |
| 127 | 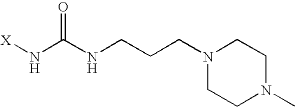 | 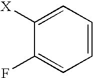 | 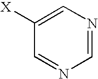 | 248.9 | | |
| 128 | 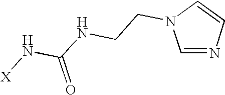 | 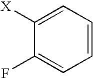 | 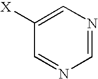 | 275.5 | | |
| 129 | 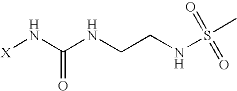 | 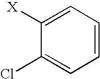 | 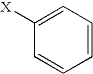 | 254.6 | | |
| 130 | 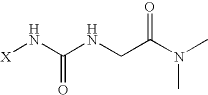 | 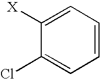 | 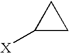 | | | |
| 131 | 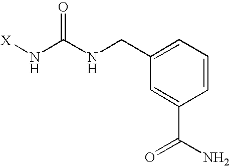 | 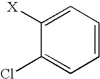 | 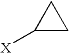 | | A | 2.85 |
| 132 | 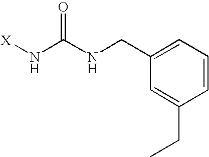 | 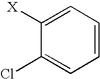 | 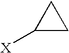 | | A | 3.50 |
| 133 | 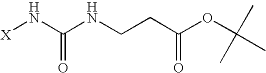 | 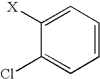 | 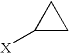 | | | |
| 134 | 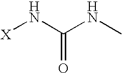 | 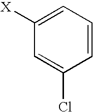 | 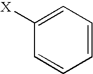 | 294.3 | | |
| 135 | 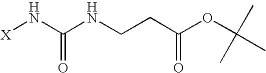 | 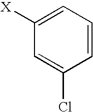 | 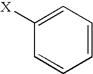 | 236.5 | | |
| 136 | 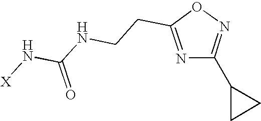 | 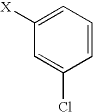 | 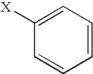 | 248.1 | | |
| 137 | 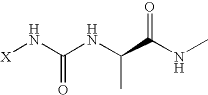 | 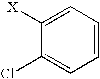 | 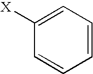 | | | |
| 138 | 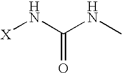 | 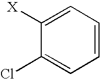 | 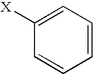 | | | |
| 139 | 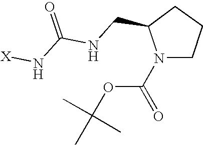 | 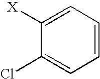 | 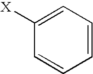 | | | |
| 140 | 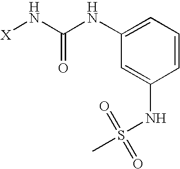 | 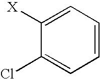 | 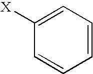 | | | |
| 141 | 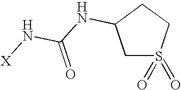 | 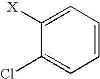 | 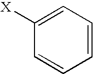 | 162.7 | | |
| 142 | 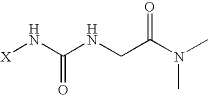 | 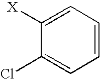 | 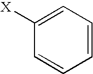 | 145.9 | | |
| 143 | 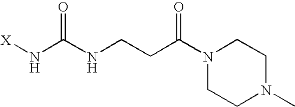 | 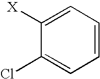 | 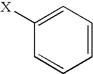 | 97.4 | | |
| 144 | 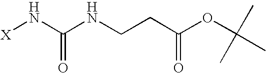 | 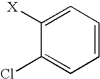 | 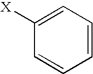 | 115.5 | | |
| 145 | 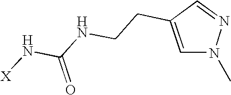 | 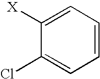 | 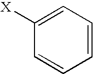 | 97.6 | | |
| 146 | 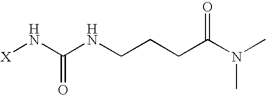 | 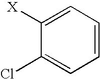 | 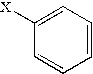 | | A | 3.14 |
| 147 | 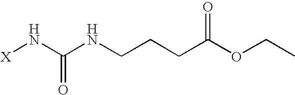 | 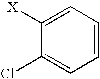 | 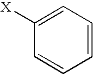 | | | |
| 148 | 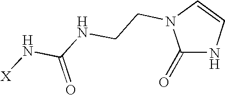 | 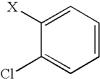 | 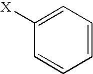 | 98.8 | | |
| 149 | 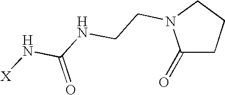 | 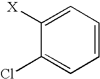 | 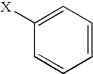 | 188.1 | | |
| 150 | 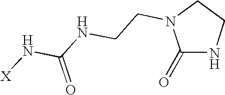 | 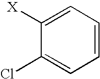 | 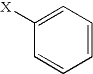 | | | |
| 151 | 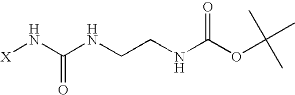 | 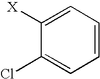 | 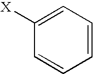 | | B | 2.21 |
| 152 | 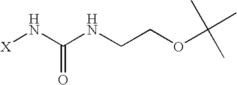 | 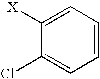 | 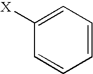 | 150.2 | | |
| 153 | 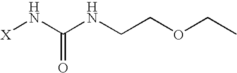 | 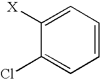 | 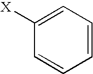 | 120.5 | | |
| 154 | 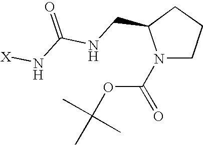 | 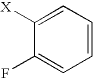 | 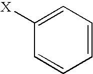 | | | |
| 155 | 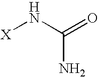 | 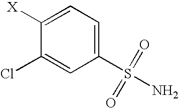 | 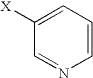 | | | |
| 156 | 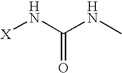 | 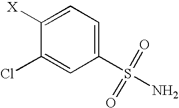 | 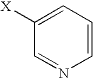 | | | |
| 157 | 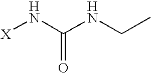 | 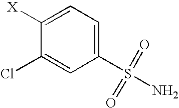 | 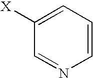 | | | |
| 158 | 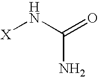 | 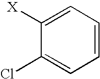 | 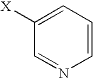 | | | |
| 159 | 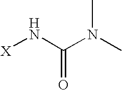 | 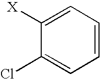 | 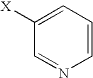 | | | |
| 160 | 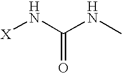 | 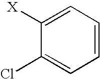 | 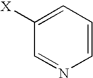 | | | |
| 161 | 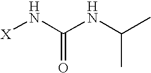 | 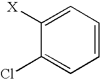 | 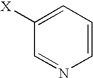 | | | |
| 162 | 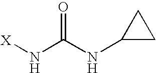 | 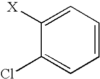 | 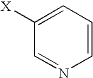 | | | |
| 163 | 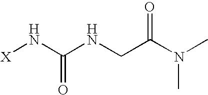 | 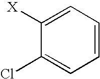 | 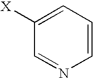 | | A | 2.22 |
| 164 | 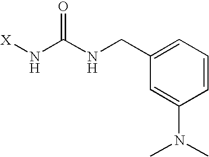 | 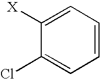 | 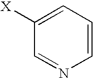 | | | |
| 165 | 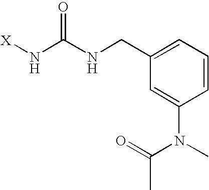 | 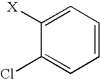 | 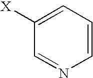 | | | |
| 166 | 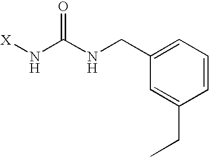 | 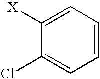 | 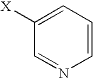 | | A | 2.79 |
| 167 | 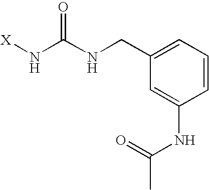 | 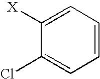 | 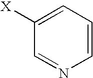 | | | |
| 168 | 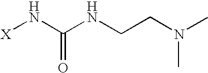 | 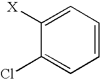 | 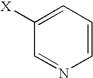 | | | |
| 169 | 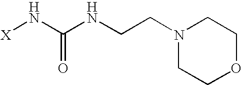 | 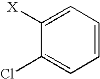 | 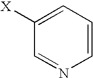 | | | |
| 170 | 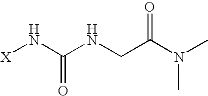 | 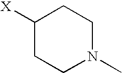 | 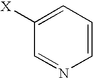 | | A | 1.88 |
| 171 | 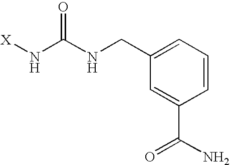 | 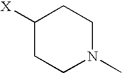 | 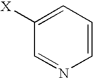 | | A | 1.97 |
| 172 | 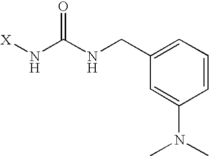 | 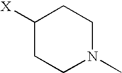 | 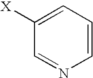 | | | |
| 173 | 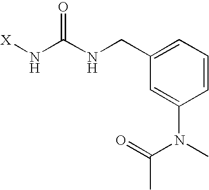 | 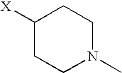 | 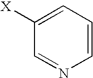 | | | |
| 174 | 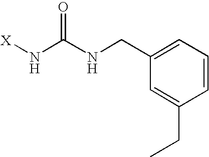 | 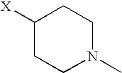 | 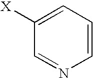 | | A | 2.30 |
| 175 | 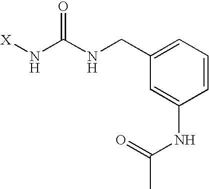 | 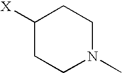 | 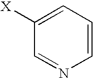 | | | |
| 176 | 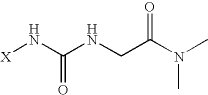 | 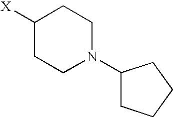 | 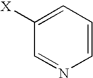 | | A | 1.96 |
| 177 | 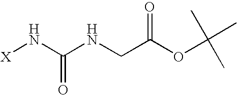 | 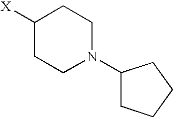 | 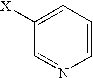 | | A | 2.27 |
| 178 | 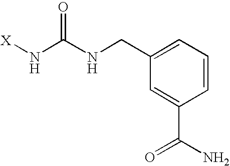 | 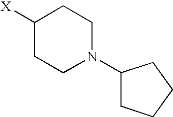 | 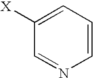 | | A | 2.10 |
| 179 | 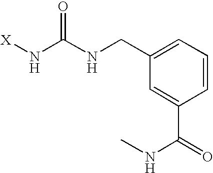 | 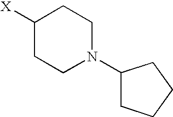 | 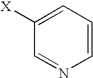 | | A | 2.08 |
| 180 | 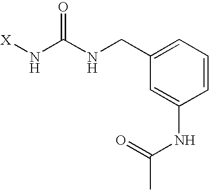 | 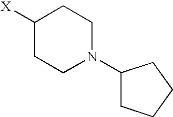 | 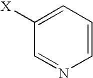 | | A | 2.18 |
| 181 | 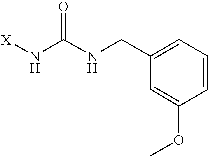 | 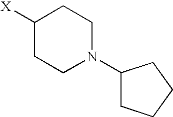 | 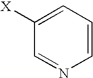 | | A | 2.31 |
| 182 | 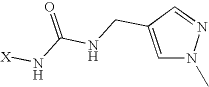 | 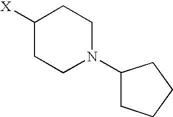 | 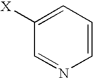 | | A | 2.00 |
| 183 | 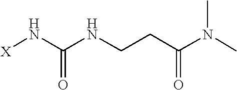 | 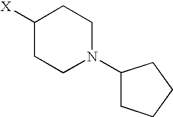 | 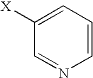 | | A | 1.98 |
| 184 | 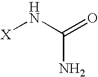 | 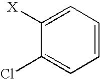 | 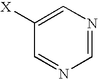 | 197.8 | | |
| 185 | 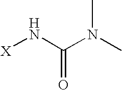 | 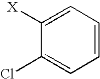 | 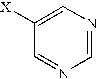 | 300.7 | | |
| 186 | 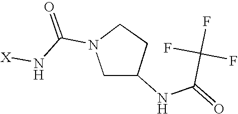 | 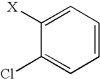 | 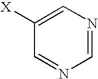 | | A | 2.92 |
| 187 | 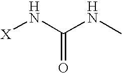 | 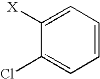 | 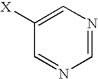 | 248.2 | | |
| 188 | 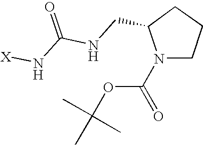 | 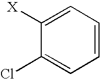 | 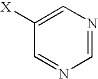 | 151.5 | | |
| 189 | 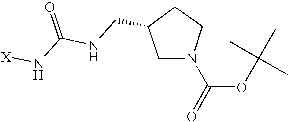 | 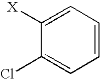 | 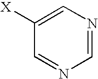 | | A | 3.17 |
| 190 | 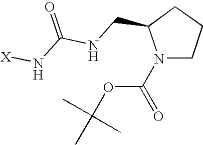 | 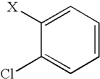 | 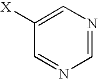 | 155.2 | | |
| 191 | 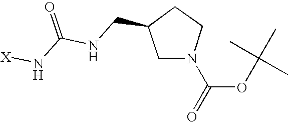 | 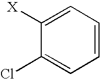 | 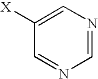 | 189.9 | | |
| 192 | 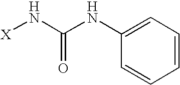 | 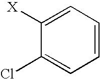 | 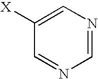 | | | |
| 193 | 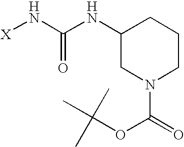 | 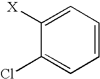 | 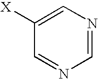 | | A | 3.17 |
| 194 | 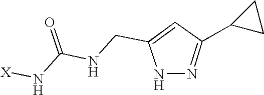 | 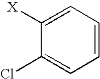 | 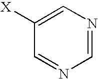 | | A | 2.58 |
| 195 | 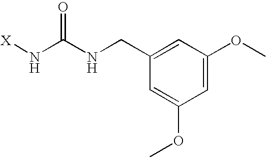 | 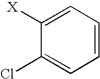 | 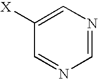 | | A | 3.14 |
| 196 | 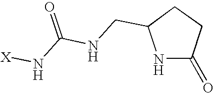 | 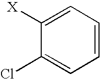 | 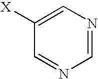 | | A | 2.45 |
| 197 | 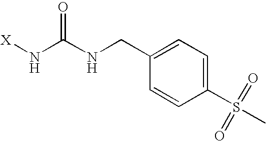 | 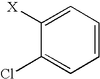 | 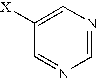 | | A | 2.82 |
| 198 | 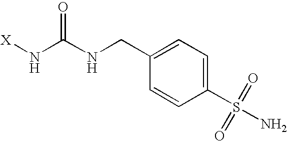 | 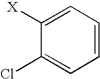 | 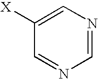 | | B | 1.80 |
| 199 | 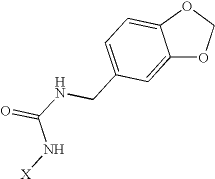 | 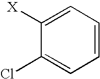 | 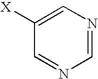 | | A | 3.11 |
| 200 | 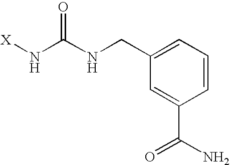 | 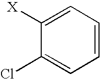 | 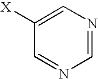 | | A | 2.64 |
| 201 | 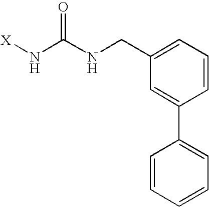 | 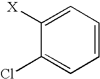 | 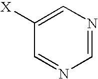 | | B | 2.30 |
| 202 | 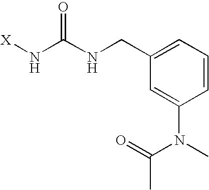 | 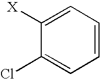 | 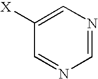 | | A | 2.85 |
| 203 | 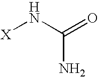 | 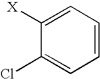 | 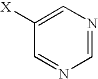 | | A | 2.85 |
| 204 | 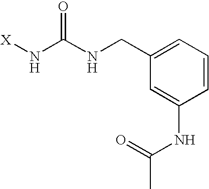 | 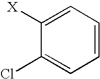 | 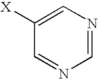 | | B | 1.84 |
| 205 | 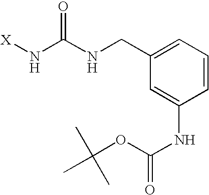 | 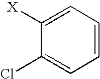 | 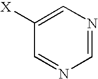 | | A | 3.32 |
| 206 | 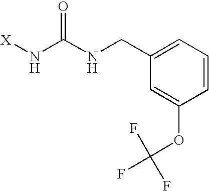 | 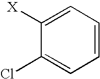 | 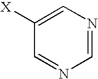 | | A | 3.40 |
| 207 | 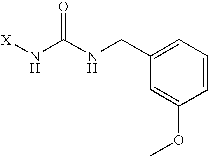 | 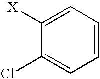 | 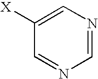 | | B | 2.07 |
| 208 | 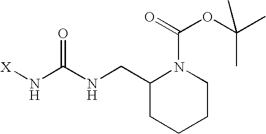 | 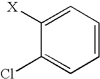 | 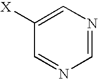 | | | |
| 209 | 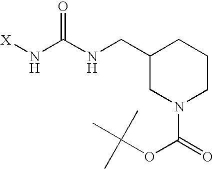 | 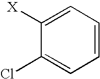 | 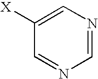 | | A | 3.27 |
| 210 | 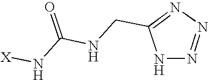 | 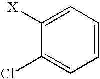 | 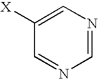 | | A | 2.50 |
| 211 | 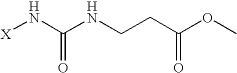 | 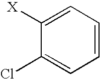 | 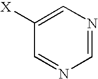 | | A | 2.79 |
| 212 | 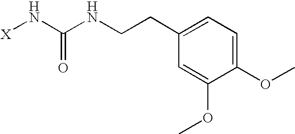 | 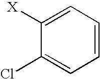 | 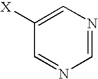 | | | |
| 213 | 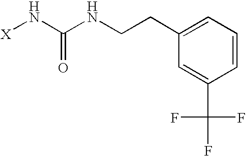 | 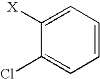 | 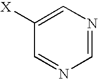 | | | |
| 214 | 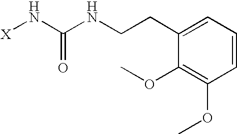 | 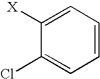 | 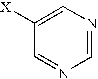 | | | |
| 215 | 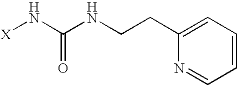 | 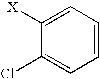 | 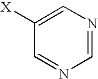 | | | |
| 216 | 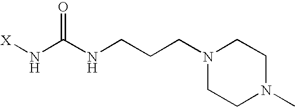 | 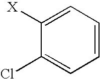 | 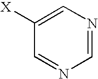 | 246.1 | | |
| 217 | 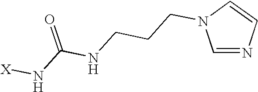 | 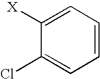 | 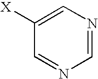 | 240.0 | | |
| 218 | 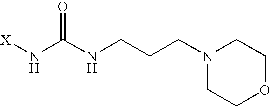 | 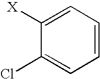 | 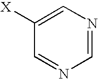 | 199.5 | | |
| 219 | 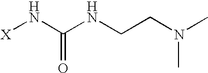 | 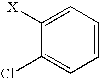 | 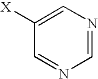 | 218.7 | A | 2.29 |
| 220 | 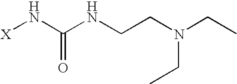 | 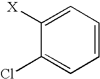 | 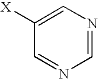 | 213.4 | | |
| 221 | 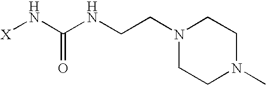 | 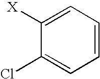 | 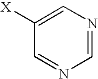 | 199.5 | | |
| 222 | 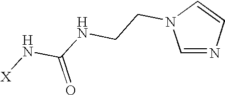 | 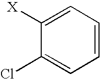 | 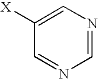 | 264.9 | | |
| 223 | 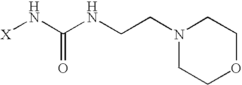 | 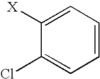 | 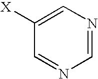 | 172.0 | | |
| 224 | 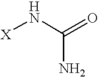 | 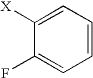 | 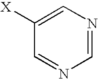 | >300 | | |
| 225 | 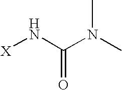 | 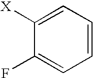 | 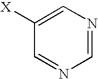 | >300 | | |
| 226 | 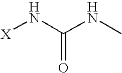 | 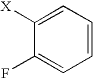 | 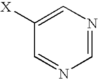 | >300 | | |
| 227 | 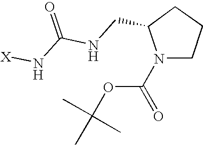 | 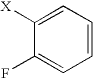 | 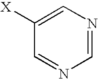 | 158.6 | | |
| 228 | 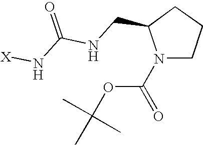 | 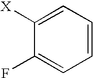 | 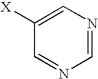 | 251.4 | | |
| 229 | 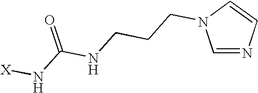 | 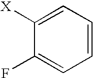 | 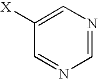 | 245.1 | | |
| 230 | 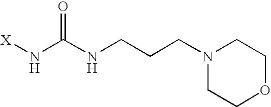 | 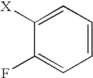 | 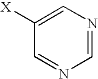 | 205.6 | | |
| 231 | 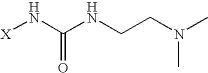 | 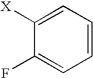 | 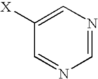 | 165.2 | | |
| 232 | 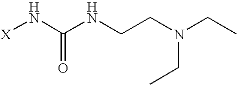 | 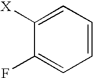 | 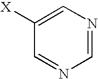 | 155.0 | | |
| 233 | 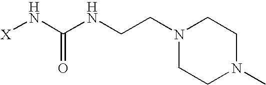 | 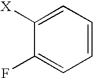 | 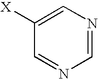 | 168.8 | | |
| 234 | 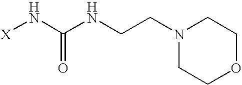 | 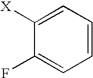 | 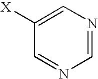 | 271.1 | | |
| 235 | 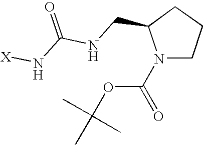 | 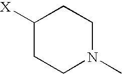 | 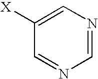 | | A | 2.39 |
| 236 | 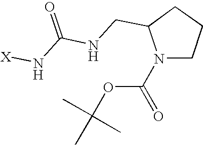 | 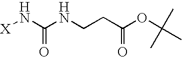 | 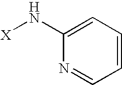 | | A | 2.90 |
| 237 | 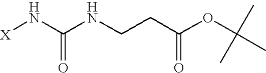 | 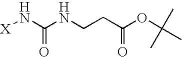 | 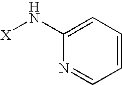 | | A | 2.83 |
| 238 | 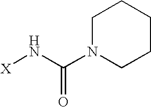 | 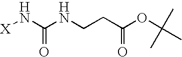 | H | | | |
| 239 | 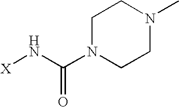 | 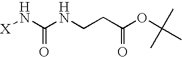 | H | | | |
| 240 | 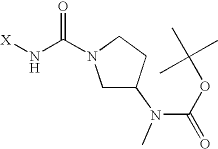 | 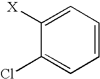 | 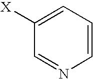 | | C | 4.03 |
| 241 | 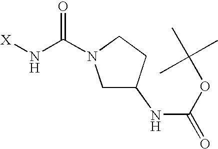 | 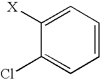 | 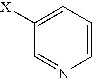 | | C | 3.81 |
| 242 | 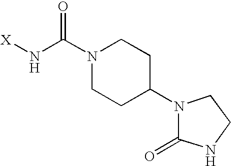 | 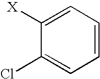 | 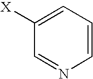 | | C | 3.25 |
| 243 | 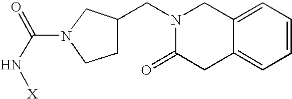 | 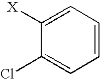 | 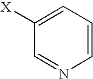 | | C | 3.81 |
| 244 | 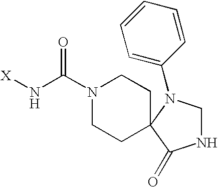 | 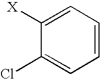 | 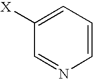 | | C | 3.77 |
| 245 | 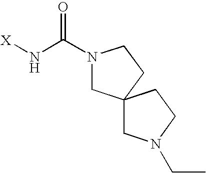 | 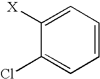 | 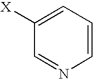 | | C | 2.95 |
| 246 | 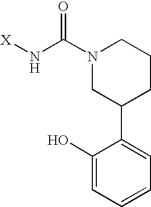 | 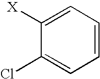 | 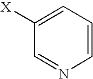 | | C | 4.09 |
| 247 | 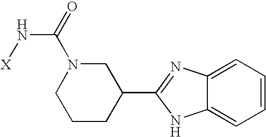 | 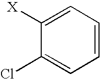 | 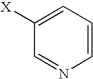 | | C | 3.21 |
| 248 | 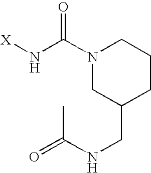 | 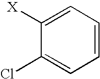 | 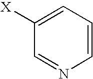 | | C | 3.35 |
| 249 | 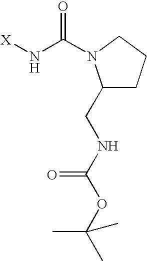 | 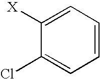 | 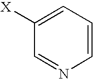 | | C | 3.96 |
| 250 | 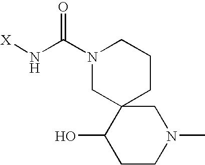 | 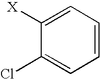 | 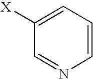 | | C | 2.94 |
| 251 | 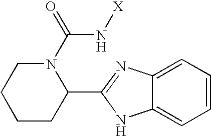 | 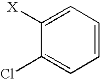 | 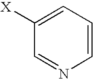 | | C | 3.24 |
| 252 | 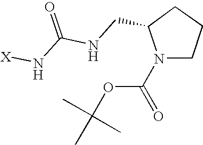 | 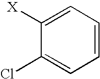 | 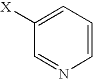 | | C | 4.02 |
| 253 | 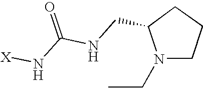 | 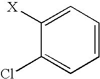 | 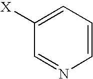 | | C | 2.96 |
| 254 | 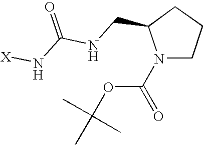 | 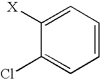 | 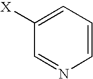 | | C | 4.02 |
| 255 | 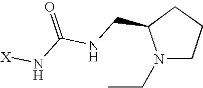 | 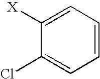 | 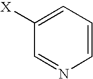 | | C | 2.97 |
| 256 | 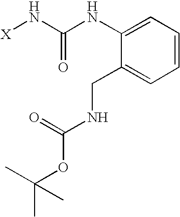 | 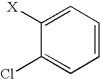 | 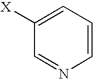 | | C | 4.19 |
| 257 | 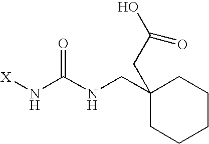 | 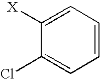 | 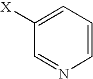 | | C | 3.84 |
| 258 | 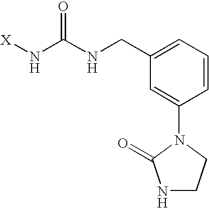 | 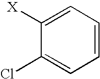 | 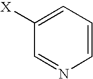 | | C | 3.5 |
| 259 | 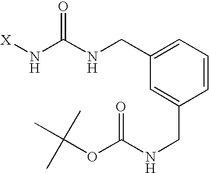 | 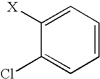 | 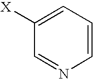 | | C | 4.05 |
| 260 | 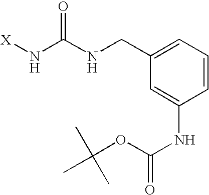 | 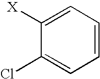 | 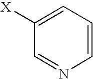 | | C | 4.12 |
| 261 | 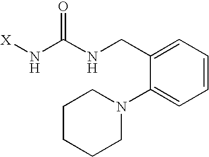 | 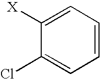 | 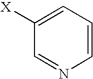 | | C | 3.27 |
| 262 | 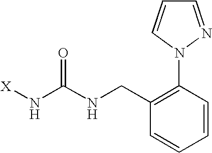 | 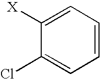 | 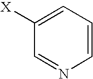 | | C | 3.86 |
| 263 | 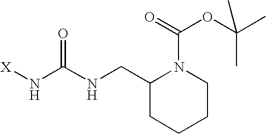 | 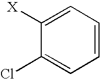 | 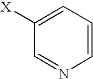 | | C | 4.07 |
| 264 | 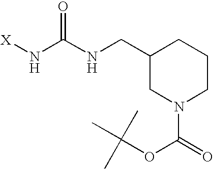 | 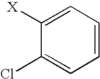 | 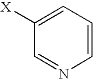 | | C | 4.11 |
| 265 | 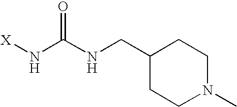 | 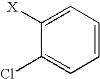 | 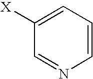 | | C | 2.89 |
| 266 | 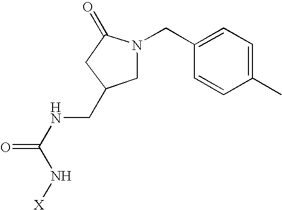 | 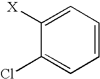 | 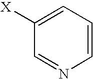 | | C | 3.76 |
| 267 | 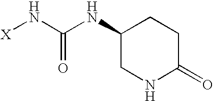 | 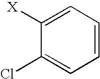 | 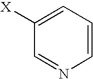 | | C | 3.08 |
| 268 | 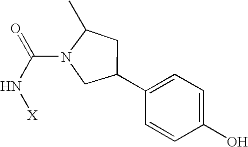 | 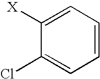 | 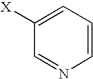 | | C | 3.9 |
| 269 | 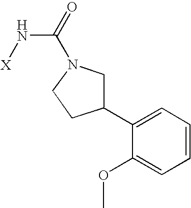 | 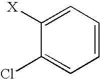 | 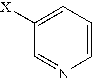 | | C | 4.24 |
| 270 | 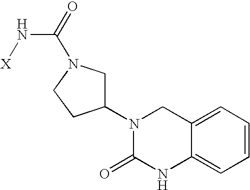 | 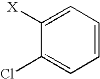 | 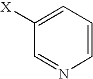 | | C | 3.7 |
| 271 | 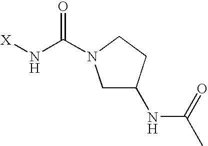 | 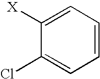 | 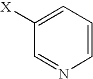 | | C | 3.12 |
| 272 | 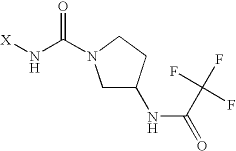 | 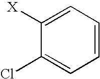 | 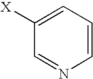 | | C | 3.57 |
| 273 | 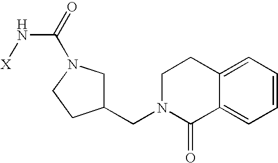 | 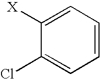 | 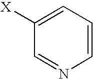 | | C | 3.88 |
| 274 | 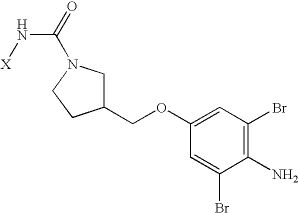 | 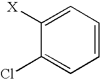 | 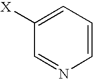 | | C | 4.43 |
| 275 | 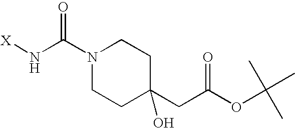 | 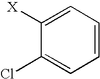 | 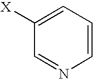 | | C | 3.85 |
| 276 | 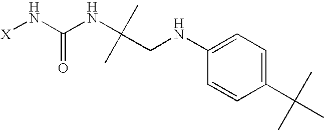 | 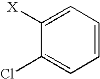 | 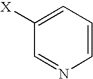 | | C | 3.93 |
| 277 | 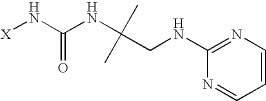 | 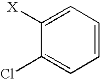 | 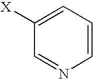 | | C | 3.26 |
| 278 | 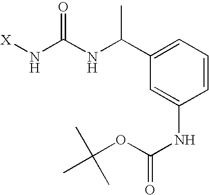 | 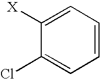 | 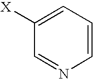 | | C | 4.22 |
| 279 | 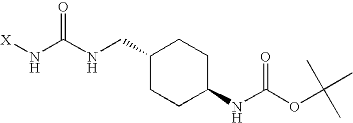 | 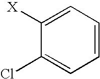 | 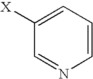 | | C | 4.11 |
| 280 | 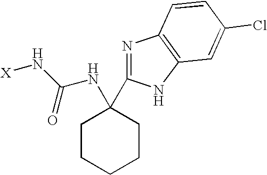 | 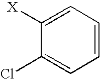 | 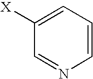 | | C | 3.51 |
| 281 | 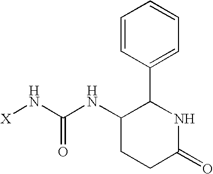 | 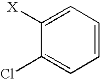 | 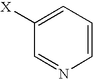 | | C | 3.41 |
| 282 | 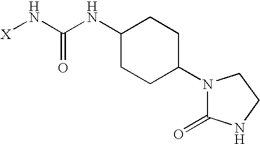 | 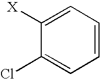 | 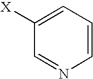 | | C | 3.38 |
| 283 | 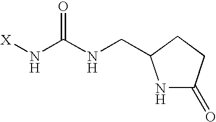 | 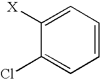 | 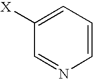 | | C | 3.12 |
| 284 | 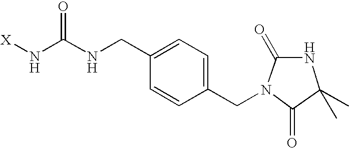 | 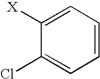 | 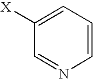 | | C | 3.6 |
| 285 | 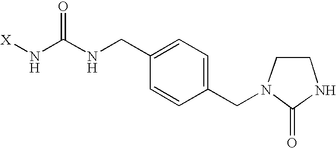 | 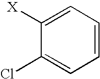 | 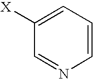 | | C | 3.48 |
| 286 | 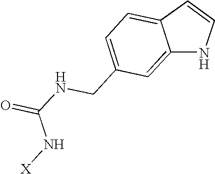 | 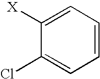 | 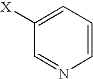 | | C | 3.85 |
| 287 | 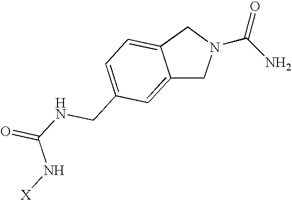 | 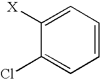 | 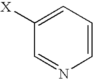 | | C | 3.34 |
| 288 | 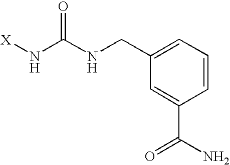 | 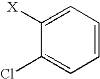 | 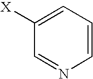 | | C | 3.31 |
| 289 | 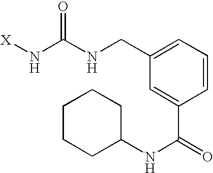 | 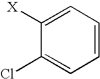 | 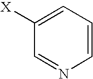 | | C | 4.02 |
| 290 | 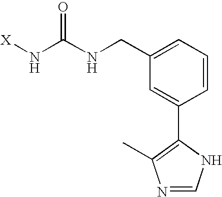 | 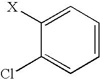 | 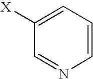 | | C | 3.2 |
| 291 | 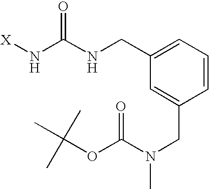 | 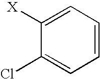 | 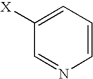 | | C | 4.2 |
| 292 | 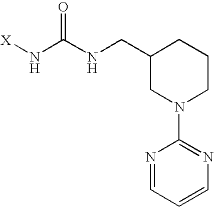 | 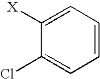 | 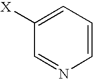 | | C | 3.31 |
| 293 | 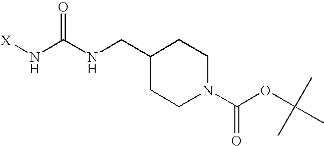 | 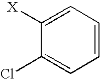 | 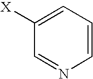 | | C | 4.08 |
| 294 | 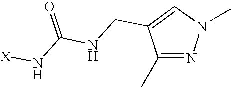 | 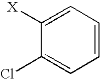 | 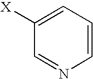 | | C | 3.23 |
| 295 | 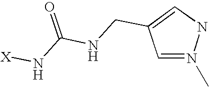 | 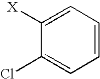 | 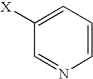 | | C | 3.27 |
| 296 | 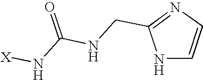 | 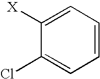 | 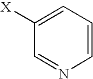 | | C | 2.8 |
| 297 | 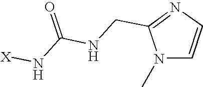 | 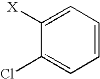 | 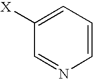 | | C | 2.86 |
| 298 | 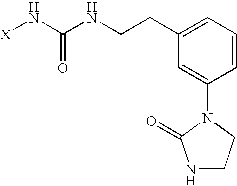 | 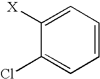 | 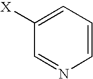 | | C | 3.61 |
| 299 | 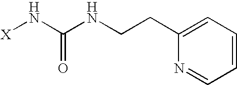 | 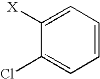 | 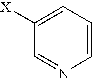 | | C | 2.88 |
| 300 | 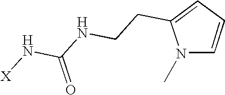 | 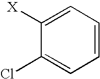 | 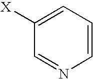 | | C | 3.77 |
| 301 | 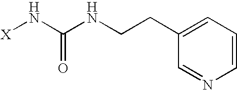 | 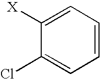 | 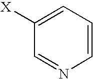 | | C | 2.9 |
| 302 | 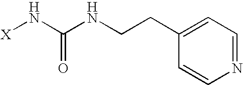 | 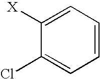 | 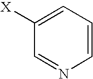 | | C | 2.9 |
| 303 | 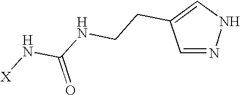 | 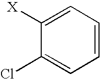 | 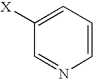 | | C | 3.09 |
| 304 | 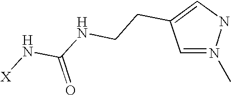 | 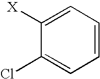 | 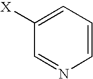 | | C | 3.31 |
| 305 | 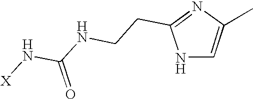 | 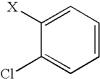 | 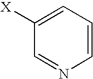 | | C | 2.9 |
| 306 | 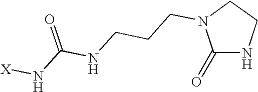 | 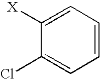 | 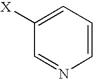 | | C | 3.22 |
| 307 | 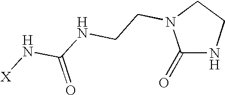 | 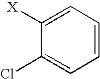 | 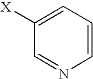 | | C | 3.13 |
| 308 | 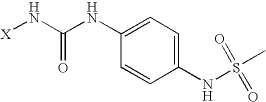 | 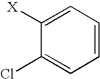 | 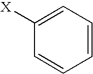 | 290.1 | | |
| 309 | 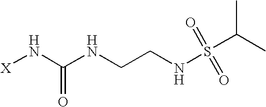 | 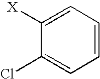 | 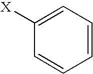 | | B | 2.12 |
| 310 | 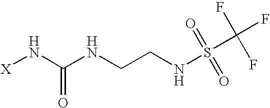 | 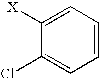 | 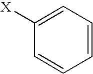 | | B | 2.36 |
| 311 | 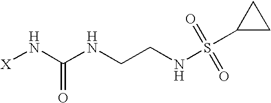 | 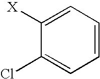 | 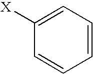 | | B | 2.11 |
| 312 | 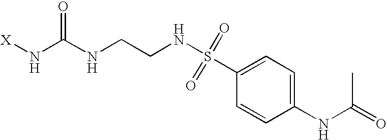 | 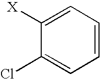 | 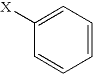 | | B | 2.06 |
| 313 | 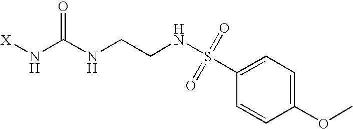 | 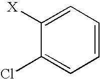 | 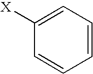 | | B | 2.21 |
| 314 | 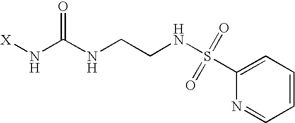 | 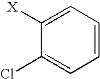 | 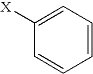 | | B | 2.09 |
| 315 | 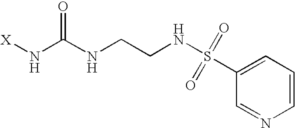 | 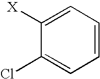 | 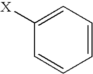 | | A | 3.15 |
| 316 | 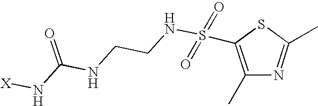 | 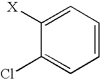 | 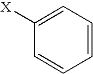 | | B | 2.14 |
| 317 | 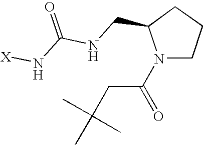 | 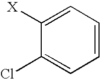 | 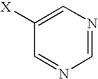 | 207.7 | | |
| 318 | 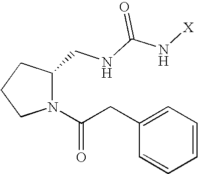 | 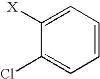 | 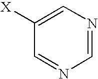 | 173.4 | | |
| 319 | 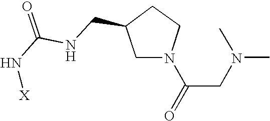 | 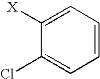 | 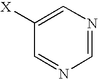 | | A | 2.31 |
| 320 | 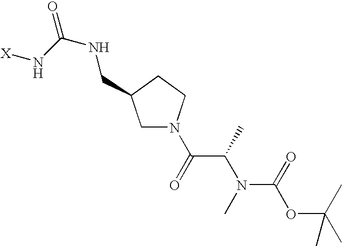 | 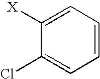 | 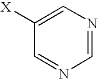 | 154.3 | | |
| 321 | 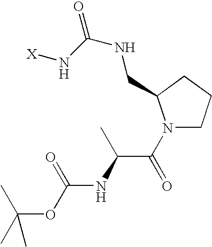 | 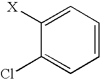 | 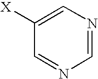 | | A | 3.15 |
| 322 | 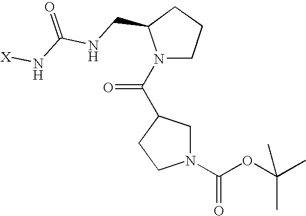 | 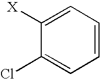 | 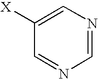 | | A | 3.10 |
| 323 | 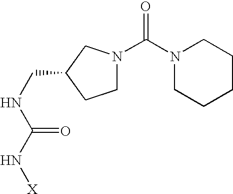 | 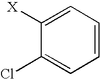 | 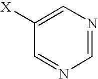 | 267.7 | | |
| 324 | 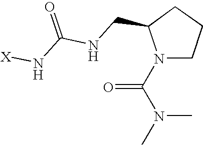 | 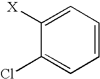 | 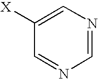 | 250.0 | | |
| 325 | 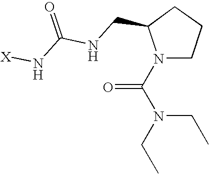 | 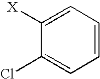 | 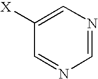 | | A | 2.88 |
| 326 | 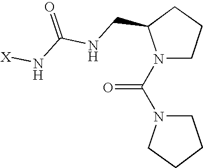 | 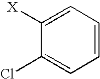 | 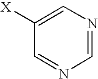 | 225.4 | | |
| 327 | 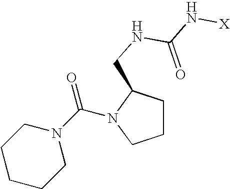 | 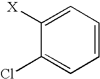 | 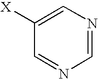 | | A | 2.91 |
| 328 | 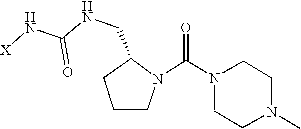 | 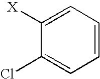 | 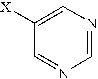 | 251.7 | | |
| 329 | 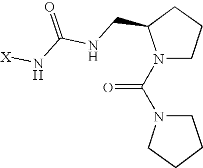 | 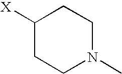 | 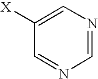 | | B | 1.49 |
| 330 | 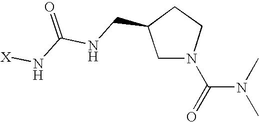 | 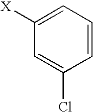 | 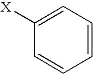 | 151.6 | | |
| 331 | 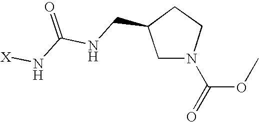 | 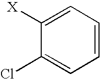 | 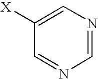 | 159.5 | | |
| 332 | 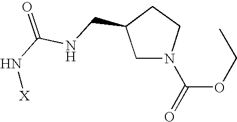 | 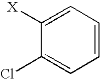 | 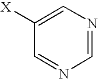 | 145.7 | | |
| 333 | 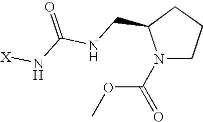 | 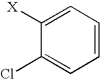 | 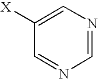 | | A | 2.97 |
| 334 | 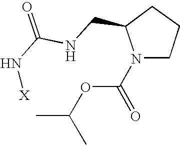 | 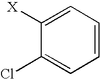 | 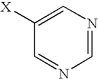 | | | |
| 335 | 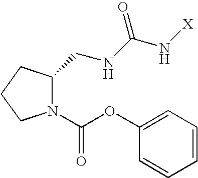 | 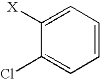 | 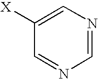 | | | |
| 336 | 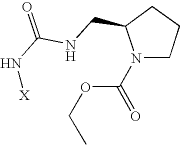 | 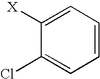 | 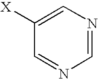 | | A | 2.95 |
| 337 | 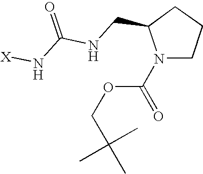 | 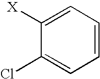 | 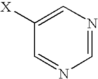 | | | |
| 338 | 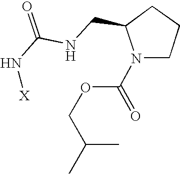 | 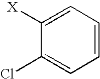 | 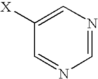 | | | |
| 339 | 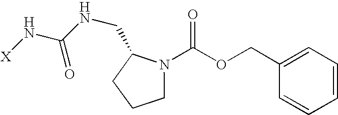 | 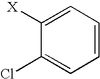 | 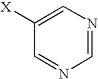 | 206.9 | | |
| 340 | 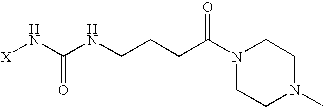 | 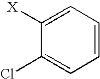 | 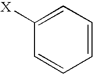 | | A | 2.72 |
| 341 | 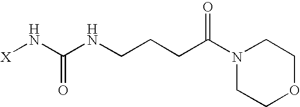 | 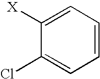 | 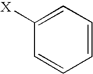 | | A | 3.10 |
| 342 | 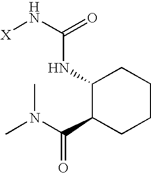 | 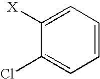 | 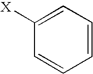 | | A | 3.36 |
| 343 | 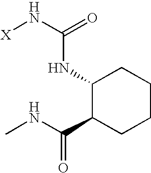 | 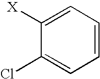 | 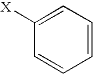 | | A | 3.17 |
| 344 | 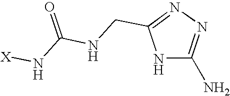 | 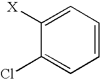 | 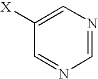 | | B | 1.57 |
| 345 | 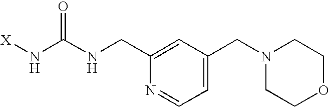 | 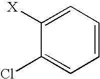 | 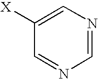 | | B | 1.54 |
| 346 | 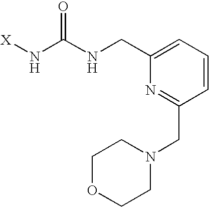 | 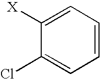 | 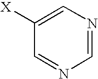 | | B | 1.69 |
| 347 | 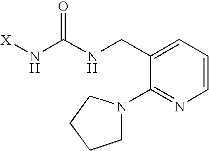 | 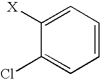 | 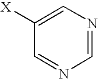 | | B | 1.75 |

Biological Test

The compounds of formula (I) mentioned by way of example are characterised by an affinity for PI3-kinase, i.e. in the test by an IC₅₀ value of below 800 nmol/litre.

In order to determine the inhibitory activity of the compounds on P13Kγ, an in-vitro kinase assay was used. The expression and purification of Gβ₁γ₂-His and p101-GST/p110γ from Sf9-cells (Spodoptera frugiperda 9) has already been described (Maier et al., J. Biol. Chem. 1999 (274) 29311-29317). Alternatively, the following method was used to determine the activity:

10 μl of the compound to be tested were placed on 96 well PVDF filter plates (0.45 μM) and incubated for 20 min with 30 μl lipid vesicles (PIP₂ (0.7 μg/well), phosphatidylethanolamine (7.5 μg/well), phosphatidylserine (7.5 μg/well), sphingomyelin (0.7 μg/well) and phosphatidylcholine (3.2 μg/well)) which contained 1-3 ng P13K□ and 20-60 ng G□₁□₂-His. The reaction was started by the addition of 10 μl reaction buffer (40 mM Hepes, pH 7.5, 100 mM NaCl, 1 mM EGTA, 1 mM □-glycerophosphate, 1 mM DTT, 7 mM MgCl₂ and 0.1% BSA; 1 μM ATP and 0.2 μCi [□-³³P]-ATP) and incubated for 120 min at ambient temperature. The reaction solution was sucked through the filters by the application of a vacuum and washed with 200 μl PBS. After the plates had been dried at 50° C. the radioactivity remaining in the plates was determined after the addition of 50 μl scintillation liquid using a Top-Count measuring device.

Ranges of Indications

It has been found that the compounds of formula (I) are characterised by a variety of possible applications in the therapeutic field. Particular mention should be made of those applications for which the compounds of formula (I) according to the invention are preferably used by virtue of their pharmaceutical activity as PI3-kinase modulators.

Generally speaking, these are diseases in whose pathology PI3-kinases are implicated, particularly inflammatory and allergic diseases. Particular mention should be made of inflammatory and allergic respiratory complaints, inflammatory diseases of the gastrointestinal tract, inflammatory diseases of the motor apparatus, inflammatory and allergic skin diseases, inflammatory eye diseases, diseases of the nasal mucosa, inflammatory or allergic ailments which involve autoimmune reactions or inflammation of the kidneys. The treatment may be symptomatic, adaptive, curative or preventative.

Respiratory complaints deserving special mention would be chronic and/or obstructive respiratory complaints. The compounds of formula 1 according to the invention may, by virtue of their pharmacological properties, bring about a reduction in
Tissue damage
Inflammation of the airways
bronchial hyperreactivity
the process of reconstruction of the lung as a result of inflammation
worsening of the disease (progression).

The compounds according to the invention are particularly preferred for preparing a medicament for the treatment of chronic bronchitis, acute bronchitis, bronchitis caused by bacterial or viral infection or fungi or helminths, allergic bronchitis, toxic bronchitis, chronic obstructive pulmonary disease (COPD), asthma (intrinsic or allergic), paediatric asthma, bronchiectasis, allergic alveolitis, allergic or non-allergic rhinitis, chronic sinusitis, cystic fibrosis or mucoviscidosis, alpha-1-antitrypsin deficiency, cough, pulmonary emphysema, interstitial lung diseases such as e.g. pulmonary fibrosis, asbestosis and silicosis and alveolitis; hyperreactive airways, nasal polyps, pulmonary oedema such as e.g. toxic pulmonary oedema and ARDS/IRDS, pneumonitis of different origins, e.g. radiation-induced or caused by aspiration or infectious pneumonitis, collagenoses such as lupus erythematodes, systemic sclerodermy, sarcoidosis or Boeck's disease.

The compounds of formula (I) are also suitable for the treatment of diseases of the skin, such as e.g. psoriasis, contact dermatitis, atopic dermatitis, alopecia areata (circular hair loss), erythema exsudativum multiforme (Stevens-Johnson Syndrome), dermatitis herpetiformis, sclerodermy, vitiligo, nettle rash (urticaria), lupus erythematodes, follicular and surface pyodermy, endogenous and exogenous acne, acne rosacea and other inflammatory or allergic or proliferative skin diseases.

Moreover, the compounds of formula (I) are suitable for therapeutic use in cases of inflammatory or allergic complaints which involve autoimmune reactions, such as e.g. inflammatory bowel diseases, e.g. Crohn's disease or ulcerative colitis; diseases of the arthritis type, such as e.g. rheumatoid or psoriatic arthritis, osteoarthritis, rheumatoid spondylitis and other arthritic conditions or multiple sclerosis.

The following general inflammatory or allergic diseases may also be mentioned, which can be treated with medicaments containing compounds of formula (I):
inflammation of the eye, such as e.g. conjunctivitis of various kinds, e.g. caused by infections with fungi or bacteria, allergic conjunctivitis, irritable conjunctivitis, drug-induced conjunctivitis, keratitis, uveitis
diseases of the nasal mucosa, such as e.g. allergic rhinitis/sinusitis or nasal polyps
inflammatory or allergic conditions, such as e.g. systemic lupus erythematodes, chronic hepatitis, kidney inflammations such as glomerulonephritis, interstitial nephritis or idiopathic nephrotic syndrome.

Other diseases which may be treated with a drug containing compounds of formula (I) on the basis of their pharmacological activity include toxic or septic shock syndrome, atherosclerosis, middle ear infections (otitis media), hypertrophy of the heart, cardiac insufficiency, stroke, ischaemic reperfusion injury or neurodegenerative diseases such as Parkinson's disease or Alzheimer's.

Combinations

The compounds of formula (I) may be used on their own or in combination with other active substances of formula (I). If desired the compounds of formula (I) may also be used in combination with W, where W denotes a pharmacologically active substance and (for example) is selected from among the betamimetics, anticholinergics, corticosteroids, PDE4-inhibitors, LTD4-antagonists, EGFR-inhibitors, dopamine agonists, H1-antihistamines, PAF-antagonists and PI3-kinase inhibitors, preferably P13-\tilde{□}Kinase inhibitors. Moreover, double or triple combinations of W may be combined with the compounds of formula (I). Combinations of W might be, for example:
W denotes a betamimetic, combined with an active substance selected from among the anticholinergics, corticosteroids, PDE4-inhibitors, EGFR-inhibitors and LTD4-antagonists,
W denotes an anticholinergic, combined with an active substance selected from among the betamimetics, corticosteroids, PDE4-inhibitors EGFR-inhibitors and LTD4-antagonists,
W denotes a corticosteroid, combined with an active substance selected from among the PDE4-inhibitors, EGFR-inhibitors and LTD4-antagonists
W denotes a PDE4-inhibitor, combined with an active substance selected from among the EGFR-inhibitors and LTD4-antagonists
W denotes an EGFR-inhibitor, combined with an LTD4-antagonist.

The compounds used as betamimetics are preferably compounds selected from among albuterol, arformoterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sulphonterol, terbutaline, tiaramide, tolubuterol, zinterol, CHF-1035, HOKU-81, KUL-1248 and
3-(4-{6-[2-hydroxy-2-(4-hydroxy-3-hydroxymethyl-phenyl)-ethylamino]-hexyloxy}-butyl)-benzyl-sulphonamide
5-[2-(5.6-diethyl-indan-2-ylamino)-1-hydroxy-ethyl]-8-hydroxy-1H-quinoline-2-one
4-hydroxy-7-[2-{[2-{[3-(2-phenylethoxy)propyl]sulphonyl}ethyl]-amino}ethyl]-2(3H)-benzothiazolone
1-(2-fluoro-4-hydroxyphenyl)-2-[4-(1-benzimidazolyl)-2-methyl-2-butylamino]ethanol
1-[3-(4-methoxybenzyl-amino)-4-hydroxyphenyl]-2-[4-(1-benzimidazolyl )-2-methyl-2-butylamino]ethanol
1-[2H-5-hydroxy-3-oxo-4H-1,4-benzoxazin-8-yl]-2-[3-(4-N,N-dimethylaminophenyl)-2-methyl-2-propylamino]ethanol
1-[2H-5-hydroxy-3-oxo-4H-1,4-benzoxazin-8-yl]-2-[3-(4-methoxyphenyl)-2-methyl-2-propylamino]ethanol
1-[2H-5-hydroxy-3-oxo-4H-1,4-benzoxazin-8-yl]-2-[3-(4-n-butyloxyphenyl)-2-methyl-2-propylamino]ethanol
1-[2H-5-hydroxy-3-oxo-4H-1,4-benzoxazin-8-yl]-2-{4-[3-(4-methoxyphenyl)-1,2,4-triazole-3-yl]-2-methyl-2-butylamino}ethanol
5-hydroxy-8-(1-hydroxy-2-isopropylaminobutyl)-2H-1,4-benzoxazin-3-(4H)-on
1-(4-amino-3-chloro-5-trifluoromethylphenyl)-2-tert.-butylamino)ethanol
6-hydroxy-8-{1-hydroxy-2-[2-(4-methoxy-phenyl)-1,1-dimethyl-ethylamino]-ethyl}-4H-benzo[1,4]oxazin-3-one
6-hydroxy-8-{1-hydroxy-2-[2-(ethyl 4-phenoxy-acetate)-1,1-dimethyl-ethylamino]-ethyl}-4H-benzo[1,4]oxazin-3-one
6-hydroxy-8-{1-hydroxy-2-[2-(4-phenoxy-acetic acid)-1,1-dimethyl-ethylamino]-ethyl}-4H-benzo[1,4]oxazin-3-one
8-{2-[1,1-dimethyl-2-(2.4.6-trimethylphenyl)-ethylamino]-1-hydroxy-ethyl}-6-hydroxy-4H-benzo[1,4]oxazin-3-one
6-hydroxy-8-{1-hydroxy-2-[2-(4-hydroxy-phenyl )-1,1-dimethyl-ethylamino]-ethyl}-4H-benzo[1,4]oxazin-3-one
6-hydroxy-8-{1-hydroxy-2-[2-(4-isopropyl-phenyl)-1.1 dimethyl-ethylamino]-ethyl}-4H-benzo[1,4]oxazin-3-one
8-{2-[2-(4-ethyl-phenyl)-1,1-dimethyl-ethylamino]-1-hydroxy-ethyl}-6-hydroxy-4H-benzo[1,4]oxazin-3-one
8-{2-[2-(4-ethoxy-phenyl)-1,1-dimethyl-ethylamino]-1-hydroxy-ethyl}-6-hydroxy-4H-benzo[1,4]oxazin-3-one
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid
8-{2-[2-(3,4-difluoro-phenyl)-1,1-dimethyl-ethylamino]-1-hydroxy-ethyl}-6-hydroxy-4H-benzo[1,4]oxazin-3-one
1-(4-ethoxy-carbonylamino-3-cyano-5-fluorophenyl)-2-(tert-butylamino)ethanol
   optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The anticholinergics used are preferably compounds selected from among the tiotropium salts, preferably the bromide salt, oxitropium salts, preferably the bromide salt, flutropium salts, preferably the bromide salt, ipratropium salts, preferably the bromide salt, glycopyrronium salts, preferably the bromide salt, trospium salts, preferably the chloride salt, tolterodine. In the above-mentioned salts the cations are the pharmacologically active constituents. As anions the above-mentioned salts may preferably contain the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred.

Other specified compounds are:
tropenol 2,2-diphenylpropionate methobromide
scopine 2,2-diphenylpropionate methobromide
scopine 2-fluoro-2,2-diphenylacetate methobromide
tropenol 2-fluoro-2,2-diphenylacetate methobromide
tropenol 3,3′,4,4′-tetrafluorobenzilate methobromide
scopine 3,3′,4,4′-tetrafluorobenzilate methobromide
tropenol 4,4′-difluorobenzilate methobromide
scopine 4,4′-difluorobenzilate methobromide
tropenol 3,3′-difluorobenzilate methobromide
scopine 3,3′-difluorobenzilate methobromide
tropenol 9-hydroxy-fluorene-9-carboxylate methobromide
tropenol 9-fluoro-fluorene-9-carboxylate methobromide
scopine 9-hydroxy-fluorene-9-carboxylate methobromide
scopine 9-fluoro-fluorene-9-carboxylate methobromide
tropenol 9-methyl-fluorene-9-carboxylate methobromide
scopine 9-methyl-fluorene-9-carboxylate methobromide
cyclopropyltropine benzilate methobromide
cyclopropyltropine 2,2-diphenylpropionate methobromide
cyclopropyltropine 9-hydroxy-xanthene-9-carboxylate methobromide
cyclopropyltropine 9-methyl-fluorene-9-carboxylate methobromide
cyclopropyltropine 9-methyl-xanthene-9-carboxylate methobromide
cyclopropyltropine 9-hydroxy-fluorene-9-carboxylate methobromide
cyclopropyltropine methyl 4,4′-difluorobenzilate methobromide
tropenol 9-hydroxy-xanthene-9-carboxylate methobromide
scopine 9-hydroxy-xanthene-9-carboxylate methobromide
tropenol 9-methyl-xanthene-9-carboxylate-methobromide
scopine 9-methyl-xanthene-9-carboxylate-methobromide
tropenol 9-ethyl-xanthene-9-carboxylate methobromide
tropenol 9-difluoromethyl-xanthene-9-carboxylate methobromide
scopine 9-hydroxymethyl-xanthene-9-carboxylate methobromide

As corticosteroids it is preferable to use compounds selected from among prednisolone, prednisone, butixocort propionate, flunisolide, beclomethasone, triamcinolone, budesonide, fluticasone, mometasone, ciclesonide, rofleponide, dexamethasone, betamethasone, deflazacort, RPR-106541, NS-126, ST-26 and
(S)-fluoromethyl 6,9-difluoro-17-[(2-furanylcarbonyl)oxy]-11-hydroxy-16-methyl-3-oxo-androsta-1,4-diene-17-carbothionate
(S)-(2-oxo-tetrahydro-furan-3S-yl)6,9-difluoro-11-hydroxy-16-methyl-3-oxo-17-propionyloxy-androsta-1,4-diene-17-carbothionate,
etiprednol-dichloroacetate
   optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, propionates, dihydrogen phosphates, palmitates, pivalates or furoates.

PDE4-inhibitors which may be used are preferably compounds selected from among enprofyllin, theophyllin, roflumilast, ariflo (cilomilast), tofimilast, pumafentrin, lirimilast, arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-1 1294A, CI-1018, CDC-801, CDC-3052, D-22888, YM-58997, Z-15370 and
N-(3,5-dichloro-1-oxo-pyridin-4-yl)-4-difluoromethoxy-3-cyclopropylmethoxybenzamide
(−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide
(R)-(+)-1-(4-bromobenzyl)-4-[(3-cyclopentyloxy)-4-methoxyphenyl]-2-pyrrolidone
3-(cyclopentyloxy-4-methoxyphenyl)-1-(4-N′-[N-2-cyano-S-methyl-isothioureido]benzyl)-2-pyrrolidone
cis[4-cyano-4-(3-cyclopentyloxy-4-methoxyphenyl)cyclohexane-1-carboxylic acid]
2-carbomethoxy-4-cyano-4-(3-cyclopropylmethoxy-4-difluoromethoxy-phenyl)cyclohexan-1-one
cis[4-cyano-4-(3-cyclopropylmethoxy-4-difluoromethoxyphenyl)cyclohexan-1-ol]
(R)-(+)-ethyl[4-(3-cyclopentyloxy-4-methoxyphenyl)pyrrolidin-2-ylidene]acetate
(S)-(−)-ethyl[4-(3-cyclopentyloxy-4-methoxyphenyl)pyrrolidin-2-ylidene]acetate
9-cyclopentyl-5,6-dihydro-7-ethyl-3-(2-thienyl)-9H-pyrazolo[3,4-c]-1,2,4-triazolo[4.3-a]pyridine
9-cyclopentyl-5,6-dihydro-7-ethyl-3-(tert-butyl)-9H-pyrazolo[3,4-c]-1,2,4-triazolo[4.3-a]pyridine
   optionally in the form of the racemates, enantiomers or diastereomers thereof and optionally in the form of the pharmacologically acceptable acid addition salts thereof, the solvates and/or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The LTD4-antagonists used are preferably compounds selected from among montelukast, pranlukast, zafirlukast, MCC-847 (ZD-3523), MN-001, MEN-91507 (LM-1507), VUF-5078, VUF-K-8707, L-733321 and
1-(((R)-(3-(2-(6,7-difluoro-2-quinolinyl)ethenyl)phenyl)-3-(2-(2-hydroxy-2-propyl)phenyl)thio)methylcyclopropane-acetic acid,
1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid
[2-[[2-(4-tert-butyl-2-thiazolyl)-5-benzofuranyl]oxymethyl]phenyl]acetic acid
   optionally in the form of the racemates, enantiomers or diastereomers thereof and optionally in the form of the pharmacologically acceptable acid addition salts, solvates and/or hydrates thereof. According to the invention the acid addition salts of the betamimetics are preferably selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which the LTD4-antagonists may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

EGFR-inhibitors which may be used are preferably compounds selected from among cetuximab, trastuzumab, ABX-EGF, Mab ICR-62 and
4-[(3-chloro-4-fluorophenyl)amino]-6-{[4-(morpholin-4-yl)-1-oxo-2-buten-1-yl]-amino}-7-cyclopropylmethoxy-quinazoline
4-[(3-chloro-4-fluorophenyl)amino]-6-{[4-(N,N-diethylamino)-1-oxo-2-buten-1-yl]amino}-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopropylmethoxy-quinazoline
4-[(R)-(1-phenyl-ethyl)amino]-6-{[4-(morpholin-4-yl)-1-oxo-2-buten-1-yl]amino}-7-cyclopentyloxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline
4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline
4-[(3-chloro-4-fluoro-phenyl)amino]-6-[2-((S)-6-methyl-2-oxo-morpholin-4-yl)-ethoxy]-7-methoxy-quinazoline
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopentyloxy-quinazoline
4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline
4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline
4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline
4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline
4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline
4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline
4-[(3-chloro-4-fluorophenyl)amino]-6-{[4-(N-cyclopropyl-N-methyl-amino)-1-oxo-2-buten-1-yl]amino}-7-cyclopentyloxy-quinazoline
4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline
4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline
4-[(3-ethynyl-phenyl)amino]-6.7-bis-(2-methoxy-ethoxy)-quinazoline
4-[(3-chloro-4-fluorophenyl)amino]-7-[3-(morpholin-4-yl)-propyloxy]-6-[(vinyl-carbonyl)amino]-quinazoline
4-[(R)-(1-phenyl-ethyl)amino]-6-(4-hydroxy-phenyl)-7H-pyrrolo[2,3-d]pyrimidine
3-cyano-4-[(3-chloro-4-fluorophenyl)amino]-6-{[4-(N,N-dimethylamino)-1-oxo-2-buten-1-yl]amino}-7-ethoxy-quinoline
4-{[3-chloro-4-(3-fluoro-benzyloxy)-phenyl]amino}-6-(5-{[(2-methanesulphonyl-ethyl)amino]methyl}-furan-2-yl)quinazoline
4-[(R)-(1-phenyl-ethyl)amino]-6-{[4-((R)-6-methyl-2-oxo-morpholin-4-yl)-1-oxo-2-buten-1-yl]amino}-7-methoxy-quinazoline
4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline
4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline
4-[(3-ethynyl-phenyl)amino]-6-{[4-(5.5-dimethyl-2-oxo-morpholin-4-yl)-1-oxo-2-buten-1-yl]amino}-quinazoline
4-[(3-chloro-4-fluoro-phenyl)amino]-6-[2-(2.2-dimethyl-6-oxo-morpholin-4-yl)-ethoxy]-7-methoxy-quinazoline
4-[(3-chloro-4-fluoro-phenyl)amino]-6-[2-(2.2-dimethyl-6-oxo-morpholin-4-yl)-ethoxy]-7-[(R)-(tetrahydrofuran-2-yl)methoxy]-quinazoline
4-[(3-chloro-4-fluoro-phenyl)amino]-7-[2-(2.2-dimethyl-6-oxo-morpholin-4-yl)-ethoxy]-6-[(S)-(tetrahydrofuran-2-yl)methoxy]-quinazoline
4-[(3-chloro-4-fluoro-phenyl)amino]-6-{2-[4-(2-oxo-morpholin-4-yl)-piperidin-1-yl]-ethoxy}-7-methoxy-quinazoline
4-[(3-chloro-4-fluoro-phenyl)amino]-6-[1-(tert.-butyloxycarbonyl)-piperidin-4-yloxy]-7-methoxy-quinazoline
4-[(3-chloro-4-fluoro-phenyl)amino]-6-(trans-4-amino-cyclohexan-1-yloxy)-7-methoxy-quinazoline
4-[(3-chloro-4-fluoro-phenyl)amino]-6-(trans-4-methanesulphonylamino-cyclohexan-1-yloxy)-7-methoxy-quinazoline
4-[(3-chloro-4-fluoro-phenyl)amino]-6-(tetrahydropyran-3-yloxy)-7-methoxy-quinazoline
4-[(3-chloro-4-fluoro-phenyl)amino]-6-(1-methyl-piperidin-4-yloxy)-7-methoxy-quinazoline
4-[(3-chloro-4-fluoro-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[(methoxymethyl)carbonyl]-piperidin-4-yloxy}-7-methoxy-quinazoline
4-[(3-chloro-4-fluoro-phenyl)amino]-6-(piperidin-3-yloxy)-7-methoxy-quinazoline
4-[(3-chloro-4-fluoro-phenyl)amino]-6-[1-(2-acetylamino-ethyl)-piperidin-4-yloxy]-7-methoxy-quinazoline
4-[(3-chloro-4-fluoro-phenyl)amino]-6-(tetrahydropyran-4-yloxy)-7-ethoxy-quinazoline
4-[(3-chloro-4-fluoro-phenyl)amino]-6-((S)-tetrahydrofuran-3-yloxy)-7-hydroxy-quinazoline
4-[(3-chloro-4-fluoro-phenyl)amino]-6-(tetrahydropyran-4-yloxy)-7-(2-methoxy-ethoxy)-quinazoline
4-[(3-chloro-4-fluoro-phenyl)amino]-6-{trans-4-[(dimethylamino)sulphonylamino]-cyclohexan-1-yloxy}-7-methoxy-quinazoline
4-[(3-chloro-4-fluoro-phenyl)amino]-6-{trans-4-[(morpholin-4-yl)carbonylamino]-cyclohexan-1-yloxy}-7-methoxy-quinazoline
4-[(3-chloro-4-fluoro-phenyl)amino]-6-{trans-4-[(morpholin-4-yl)sulphonylamino]-cyclohexan-1-yloxy}-7-methoxy-quinazoline
4-[(3-chloro-4-fluoro-phenyl)amino]-6-(tetrahydropyran-4-yloxy)-7-(2-acetylamino-ethoxy)-quinazoline
4-[(3-chloro-4-fluoro-phenyl)amino]-6-(tetrahydropyran-4-yloxy)-7-(2-methanesulphonylamino-ethoxy)-quinazoline
4-[(3-chloro-4-fluoro-phenyl)amino]-6-{1-[(piperidin-1-yl)carbonyl]-piperidin-4-yloxy}-7-methoxy-quinazoline
4-[(3-chloro-4-fluoro-phenyl)amino]-6-(1-aminocarbonylmethyl-piperidin-4-yloxy)-7-methoxy-quinazoline
4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline
4-[(3-chloro-4-fluoro-phenyl)amino]-6-(trans-4-ethansulphonylamino-cyclohexan-1-yloxy)-7-methoxy-quinazoline
4-[(3-chloro-4-fluoro-phenyl)amino]-6-(1-methanesulphonyl-piperidin-4-yloxy)-7-ethoxy-quinazoline
4-[(3-chloro-4-fluoro-phenyl)amino]-6-(1-methanesulphonyl-piperidin-4-yloxy)-7-(2-methoxy-ethoxy)-quinazoline
4-[(3-chloro-4-fluoro-phenyl)amino]-6-[1-(2-methoxy-acetyl)-piperidin-4-yloxy]-7-(2-methoxy-ethoxy)-quinazoline
4-[(3-chloro-4-fluoro-phenyl)amino]-6-(cis-4-acetylamino-cyclohexan-1-yloxy)-7-methoxy-quinazoline
4-[(3-ethynyl-phenyl)amino]-6-[1-(tert.-butyloxycarbonyl)-piperidin-4-yloxy]-7-methoxy-quinazoline
4-[(3-ethynyl-phenyl)amino]-6-(tetrahydropyran-4-yloxy]-7-methoxy-quinazoline
4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline
4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline
4-[(3-chloro-4-fluoro-phenyl)amino]-6-{cis-4-[(morpholin-4-yl)carbonylamino]-cyclohexan-1-yloxy}-7-methoxy-quinazoline
4-[(3-chloro-4-fluoro-phenyl)amino]-6-{1-[2-(2-oxopyrrolidin-1-yl)ethyl]-piperidin-4-yloxy}-7-methoxy-quinazoline
4-[(3-chloro-4-fluoro-phenyl)amino]-6-{1-[(morpholin-4-yl)carbonyl]-piperidin-4-yloxy}-7-(2-methoxy-ethoxy)-quinazoline
4-[(3-ethynyl-phenyl)amino]-6-(1-acetyl-piperidin-4-yloxy)-7-methoxy-quinazoline
4-[(3-ethynyl-phenyl)amino]-6-(1-methyl-piperidin-4-yloxy)-7-methoxy-quinazoline
4-[(3-ethynyl-phenyl)amino]-6-(1-methanesulphonyl-piperidin-4-yloxy)-7-methoxy-quinazoline
4-[(3-chloro-4-fluoro-phenyl)amino]-6-(1-methyl-piperidin-4-yloxy)-7(2-methoxy-ethoxy)-quinazoline
4-[(3-chloro-4-fluoro-phenyl)amino]-6-(1-isopropyloxycarbonyl-piperidin-4-yloxy)-7-methoxy-quinazoline
4-[(3-chloro-4-fluoro-phenyl)amino]-6-(cis-4-methylamino-cyclohexan-1-yloxy)-7-methoxy-quinazoline
4-[(3-chloro-4-fluoro-phenyl)amino]-6-{cis-4-[N-(2-methoxy-acetyl)-N-methyl-amino]-cyclohexan-1-yloxy}-7-methoxy-quinazoline
4-[(3-ethynyl-phenyl)amino]-6-(piperidin-4-yloxy)-7-methoxy-quinazoline
4-[(3-ethynyl-phenyl)amino]-6-[1-(2-methoxy-acetyl)-piperidin-4-yloxy]-7-methoxy-quinazoline
4-[(3-ethynyl-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[(cis-2,6-dimethyl-morpholin-4-yl)carbonyl]-piperidin-4-yloxy}-7-methoxy-quinazoline
4-[(3-chloro-4-fluoro-phenyl)amino]-6-{1-[(2-methyl-morpholin-4-yl)carbonyl]-piperidin-4-yloxy}-7-methoxy-quinazoline
4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline
4-[(3-chloro-4-fluoro-phenyl)amino]-6-{1-[(N-methyl-N-2-methoxyethyl-amino)carbonyl]-piperidin-4-yloxy}-7-methoxy-quinazoline
4-[(3-chloro-4-fluoro-phenyl)amino]-6-(1-ethyl-piperidin-4-yloxy)-7-methoxy-quinazoline
4-[(3-chloro-4-fluoro-phenyl)amino]-6-{1-[(2-methoxyethyl)carbonyl]-piperidin-4-yloxy}-7-methoxy-quinazoline
4-[(3-chloro-4-fluoro-phenyl)amino]-6-{1-[(3-methoxypropyl-amino)-carbonyl]-piperidin-4-yloxy}-7-methoxy-quinazoline
4-[(3-chloro-4-fluoro-phenyl)amino]-6-[cis-4-(N-methanesulphonyl-N-methyl-amino)-cyclohexan-1-yloxy]-7-methoxy-quinazoline
4-[(3-chloro-4-fluoro-phenyl)amino]-6-[cis-4-(N-acetyl-N-methyl-amino)-cyclohexan-1-yloxy]-7-methoxy-quinazoline
4-[(3-chloro-4-fluoro-phenyl)amino]-6-(trans-4-methylamino-cyclohexan-1-yloxy)-7-methoxy-quinazoline
4-[(3-chloro-4-fluoro-phenyl)amino]-6-[trans-4-(N-methanesulphonyl-N-methyl-amino)-cyclohexan-1-yloxy]-7-methoxy-quinazoline
4-[(3-chloro-4-fluoro-phenyl)amino]-6-(trans-4-dimethylamino-cyclohexan-1-yloxy)-7-methoxy-quinazoline
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-[2-(2.2-dimethyl-6-oxo-morpholin-4-yl)-ethoxy]-7-[(S)-(tetrahydrofuran-2-yl)methoxy]-quinazoline
4-[(3-chloro-4-fluoro-phenyl)amino]-6-(1-methanesulphonyl-piperidin-4-yloxy)-7-methoxy-quinazoline
4-[(3-chloro-4-fluoro-phenyl)amino]-6-(1-cyano-piperidin-4-yloxy)-7-methoxy-quinazoline
   optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The dopamine agonists used are preferably compounds selected from among bromocriptin, cabergoline, alpha-dihydroergocryptine, lisuride, pergolide, pramipexol, roxindol, ropinirol, talipexol, tergurid and viozan, optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate.

H1-Antihistamines which may be used are preferably compounds selected from among epinastine, cetirizine, azelastine, fexofenadine, levocabastine, loratadine, mizolastine, ketotifen, emedastine, dimetindene, clemastine, bamipine, cexchlorpheniramine, pheniramine, doxylamine, chlorophenoxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The PAF-antagonists used are preferably compounds selected from among
4-(2-chlorophenyl)-9-methyl-2-[3(4-morpholinyl)-3-propanon-1-yl]-6H-thieno-[3,2-f]-[1,2,4]triazolo[4,3-a][1,4]diazepine
6-(2-chlorophenyl)-8,9-dihydro-1-methyl-8-[(4-morpholinyl)carbonyl]-4H,7H-cyclo-penta-[4,5]thieno-[3,2-f][1,2,4]triazolo[4,3-a][1,4]diazepine,
   optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The PI3-kinase-δ-inhibitors used are preferably compounds selected from among:

IC87114, 2-(6-aminopurin-9-ylmethyl)-3-(2-chlorophenyl)-6.7-dimethoxy-3H-quinazolin-4-one; 2-(6-aminopurin-o-ylmethyl)-6-bromo-3-(2-chlorophenyl )-3H-quinazolin-4-one; 2-(6-aminopurin-o-ylmethyl)-3-(2-chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chloro-phenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one;5-chloro-2-(9H-purin-6-ylsulphanylmethyl)-3-o-tolyl-3H-quinazolin-4-one; 5-chloro-3-(2-fluorophenyl)-2-(9H-purin-6-yl-sulphanylmethyl)-3H-quinazolin-4-one; 2-(6-aminopurin-9-ylmethyl)-5-chloro-3-(2-fluorophenyl)-3H-quinazolin-4-one; 3-biphenyl-2-yl-5-chloro-2-(9H-purin-6-ylsulphanylmethyl)-3H-quinazolin-4-one; 5-chloro-3-(2-methoxyphenyl)-2-(9H-purin-6-yl-sulphanylmethyl)-3H-quinazolin-4-one; 3-(2-chlorophenyl)-5-fluoro-2-(9H-purin-6-yl-sulphanylmethyl)-3H-quinazolin-4-one; 3-(2-chlorophenyl)-6.7-dimethoxy-2-(9H-purin-6-yl-sulphanylmethyl)-3H-quinazolin-4-one; 6-bromo-3-(2-chlorophenyl)-2-(9H-purin-6-yl-sulphanylmethyl)-3H-quinazolin-4-one; 3-(2-chlorophenyl)-8-trifluoromethyl-2-(9H-purin-6-ylsulphanylmethyl)-3H-quinazolin-4-one; 3-(2-chlorophenyl)-2-(9H-purin-6-ylsulphanylmethyl)-3H-benzo[g]quinazolin-4-one; 6-chloro-3-(2-chlorophenyl)-2-(9H-purin-6-yl-sulphanylmethyl)-3H-quinazolin-4-one; 8-chloro-3-(2-chlorophenyl)-2-(9H-purin-6-yl-sulphanylmethyl)-3H-quinazolin-4-one; 3-(2-chlorophenyl)-7-fluoro-2-(9H-purin-6-yl-sulphanylmethyl)-3H-quinazolin-4-one; 3-(2-chlorophenyl)-7-nitro-2-(9H-purin-6-yl-sulphanylmethyl)-3H-quinazolin-4-one; 3-(2-chlorophenyl)-6-hydroxy-2-(9H-purin-6-yl-sulphanylmethyl)-3H-quinazolin-4-one; 5-chloro-3-(2-chlorophenyl)-2-(9H-purin-6-yl-sulphanylmethyl)-3H-quinazolin-4-one; 3-(2-chlorophenyl)-5-methyl-2-(9H-purin-6-yl-sulphanylmethyl)-3H-quinazolin-4-one; 3-(2-chlorophenyl)-6.7-difluoro-2-(9H-purin-6-yl-sulphanylmethyl)-3H-quinazolin-4-one; 3-(2-chlorophenyl)-6-fluoro-2-(9H-purin-6-yl-sulphanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one; 3-(2-fluorophenyl)-5-methyl-2-(9H-purin-6-yl-sulphanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulphanylmethyl)-3-cyclopropyl-5-methyl-3H-quinazolin-4-one; 3-cyclopropylmethyl-5-methyl-2-(9H-purin-6-ylsulphanylmethyl)-3H-quinazolin-4-one; 2-(6-aminopurin-9-ylmethyl)-3-cyclopropylmethyl-5-methyl-3H-quinazolin-4-one; 2-(2-amino-9H-purin-6-ylsulphanylmethyl)-3-cyclopropylmethyl-5-methyl-3H-quinazolin-4-one; 5-methyl-3-phenethyl-2-(9H-purin-6-ylsulphanylmethyl)-3H-quinazolin-4-one; 2-(2-amino-9H-purin-6-ylsulphanylmethyl)-5-methyl-3-phenethyl-3H-quinazolin-4-one; 3-cyclopentyl-5-methyl-2-(9H-purin-6-ylsulphanylmethyl)-3H-quinazolin-4-one;2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulphanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulphanylmethyl)-4H-quinazolin-3-yl]-benzoic acid; 3-cyclopropyl-5-methyl-2-(9H-purin-6-ylsulphanylmethyl)-3H-quinazolin-4-one; 2-(6-aminopurin-9-ylmethyl)-3-cyclopropyl-5-methyl-3H-quinazolin-4-one; 5-methyl-3-(4-nitrobenzyl)-2-(9H-purin-6-ylsulphanylmethyl)-3H-quinazolin-4-one; 3-cyclohexyl-5-methyl-2-(9H-purin-6-ylsulphanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulphanylmethyl)-3-cyclohexyl-5-methyl-3H-quinazolin-4-one; 5-methyl-3-(E-2-phenylcyclopropyl)-2-(9H-purin-6-ylsulphanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6-ylamino)methyl]-5-methyl-3-o-tolyl-3H-quinazolin-4-one; 2-[(2-fluoro-9H-purin-6-ylamino)methyl]-5-methyl-3-o-tolyl-3H-quinazolin-4-one; (2-chlorophenyl)-dimethylamino-(9H-purin-6-ylsulphanylmethyl)-3H-quinazolin-4-one; 5-(2-benzyloxyethoxy)-3-(2-chlorophenyl)-2-(9H-purin-6-ylsulphanylmethyl)-3H-quinazolin-4-one; 3-(2-chlorophenyl)-5-fluoro-4-oxo-3,4-dihydro-quinazolin-2-ylmethyl 6-aminopurine-9-carboxylate; N-[3-(2-chlorophenyl)-5-fluoro-4-oxo-3,4-dihydro-quinazolin-2-ylmethyl]-2-(9H-purin-6-ylsulphanyl)-acetamide; 2-[1-(2-fluoro-9H-purin-6-ylamino)ethyl]-5-methyl-3-o-tolyl-3H-quinazolin-4-one; 5-methyl-2-[1-(9H-purin-6-ylamino)ethyl]-3-o-tolyl-3H-quinazolin-4-one; 2-(6-dimethylaminopurin-9-ylmethyl )-5-methyl-3-o-tolyl-3H-quinazolin-4-one; 5-methyl-2-(2-methyl-6-oxo-1.6-dihydro-purin-7-ylmethyl)-3-o-tolyl-3H-quinazolin-4-one; 5-methyl-2-(2-methyl-6-oxo-1.6-dihydro-purin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulphanylmethyl)-5-methyl-3-o-tolyl-3H-quinazolin-4-one; 2-(4-amino-1,3,5-triazin-2-ylsulphanylmethyl)-5-methyl-3-o-tolyl-3H-quinazolin-4-one; 5-methyl-2-(7-methyl-7H-purin-6-ylsulphanylmethyl)-3-o-tolyl-3H-quinazolin-4-one; 5-methyl-2-(2-oxo-1,2-dihydro-pyrimidin-4-ylsulphanylmethyl)-3-o-tolyl-3H-quinazolin-4-one; 5-methyl-2-purin-7-ylmethyl-3-o-tolyl-3H-quinazolin-4-one;5-methyl-2-purin-9-ylmethyl-3-o-tolyl-3H-quinazolin-4-one; 5-methyl-2-(9-methyl-9H-purin-6-ylsulphanylmethyl)-3-o-tolyl-3H-quinazolin-4-one; 2-(2,6-diamino-pyrimidin-4-ylsulphanylmethyl)-5-methyl-3-o-tolyl-3H-quinazolin-4-one; 5-methyl-2-(5-methyl-[1,2,4]triazolo[1.5-a]pyrimidin-7-ylsulphanylmethyl)-3-0-tolyl-3H-quinazolin-4-one; 5-methyl-2-(2-methylsulphanyl-9H-purin-6-ylsulphanylmethyl)-3-o-tolyl-3H-quinazolin-4-one; 2-(2-hydroxy-9H-purin-6-ylsulphanylmethyl)-5-methyl-3-o-tolyl-3H-quinazolin-4-one; 5-methyl-2-(1-methyl-1H-imidazol-2-ylsulphanylmethyl)-3-o-tolyl-3H-quinazolin-4-one; 5-methyl-3-0-tolyl-2-(H-[1,2,4]triazol-3-ylsulphanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one; 2-(6-amino-9H-purin-2-ylsulphanylmethyl)-5-methyl-3-o-tolyl-3H-quinazolin-4-one; 2-(2-amino-6-ethylamino-pyrimidin-4-ylsulphanylmethyl)-5-methyl-3-o-tolyl-3H-quinazolin-4-one; 2-(3-amino-5-methylsulphanyl-1,2,4-triazol-1-yl-methyl)-5-methyl-3-o-tolyl-3Hquinazolin-4-one; 2-(5-amino-3-methylsulphanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-yl methyl)-5-methyl-3-o-tolyl-3 H-quinazolin-4-one; 2-(2,6-diaminopurin-9-ylmethyl)-5-methyl-3-o-tolyl-3H-quinazolin-4-one; 5-methyl-2-(9H-purin-6-ylsulphanyl methyl)-3-o-tolyl-3H-quinazolin-4-one; 3-isobutyl-5-methyl-2-(9H-purin-6-ylsulphanylmethyl)-3H-quinazolin-4-one; N-{2-[5-methyl-4-oxo-2-(9H-purin-6-ylsulphanylmethyl)-4H-quinazolin-3-yl]-phenyl}-acetamide; 5-methyl-3-(E-2-methyl-cyclohexyl)-2-(9H-purin-6-ylsulphanylmethyl)-3H-quinazolin-4-one; 2-[5-methyl-4-oxo-2-(9H-purin-6-ylsulphanylmethyl)-4H-quinazolin-3-yl]-benzoic acid; 3-{2-[(2-dimethylaminoethyl)methylamino]phenyl}-5-methyl-2-(9H-purin-6-ylsulphanylmethyl)-3H-quinazolin-4-one; 3-(2-chlorophenyl)-5-methoxy-2-(9H-purin-6-ylsulphanylmethyl)-3H-quinazolin-4-one; 3-(2-chlorophenyl)-5-(2-morpholin-4-yl-ethylamino)-2-(9H-purin-6-ylsulphanylmethyl)-3H-quinazolin-4-one; 3-benzyl-5-methoxy-2-(9H-purin-6-ylsulphanylmethyl)-3H-quinazolin-4-one; 2-(6-aminopurin-9-ylmethyl)-3-(2-benzyloxyphenyl)-5-methyl-3H-quinazolin-4-one; 2-(6-aminopurin-9-ylmethyl)-3-(2-hydroxyphenyl)-5-methyl-3H-quinazolin-4-one; 2-(1-(2-amino-9H-purin-6-ylamino)ethyl)-5-methyl-3-o-tolyl-3H-quinazolin-4-one; 5-methyl-2-[1-(9H-purin-6-ylamino)propyl]-3-o-tolyl-3H-quinazolin-4-one; 2-(1-(2-fluoro-9H-purin-6-ylamino)propyl)-5-methyl-3-o-tolyl-3H-quinazolin-4-one; 2-(1-(2-amino-9H-purin-6-ylamino)propyl)-5-methyl-3-o-tolyl-3H-quinazolin-4-one; 2-(2-benzyloxy-1-(9H-purin-6-ylamino)ethyl)-5-methyl-3-o-tolyl-3H-quinazolin-4-one; 2-(6-aminopurin-9-yl methyl)-5-methyl-3-{2-(2-(1-methylpyrrolidin-2-yl)-ethoxy)-phenyl}-3H-quinazolin-4-one; 2-(6-aminopurin-9-ylmethyl)-3-(2-(3-dimethylamino-propoxy)-phenyl)-5-methyl-3H-quinazolin-4-one; 2-(6-aminopurin-9-ylmethyl)-5-methyl-3-(2-prop-2-ynyloxyphenyl)-3H-quinazolin-4-one; 2-(2-(1-(6-aminopurin-9-ylmethyl)-5-methyl-4-oxo-4H-quinazolin-3-yl]-phenoxy}-acetamide; 5-chloro-3-(3,5-difluoro-phenyl)-2-[1-(9H-purin-6-ylamino)-propyl]-3H-quinazolin-4-one; 3-phenyl-2-[1-(9H-purin-6-ylamino)-propyl]-3H-quinazolin-4-one; 5-fluoro-3-phenyl-2-[1-(9 H-purin-6-ylamino)-propyl]-3 H-quinazolin-4-one; 3-(2,6-difluoro-phenyl)-5-methyl-2-[1-(9H-purin-6-ylamino)-propyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-3-phenyl-2-[1-(9H-purin-6-ylamino)-ethyl]-3H-quinazolin-4-one; 3-(2.3-difluoro-phenyl)-5-methyl-2-[1-(9H-purin-6-ylamino)-ethyl]-3H-quinazolin-4-one; 5-methyl-3-phenyl-2-[1-(9H-purin-6-ylamino)-ethyl]-3H-quinazolin-4-one; 3-(3-chloro-phenyl)-5-methyl-2-[1-(9H-purin-6-ylamino)-ethyl]-3H-quinazolin-4-one; 5-methyl-3-phenyl-2-[(9H-purin-6-ylamino)-methyl]-3H-quinazolin-4-one; 2-[(2-amino-9H-purin-6-ylamino)-methyl]-3-(3,5-difluoro-phenyl)-5-methyl-3H-quinazolin-4-one; 3-{2-[(2]-diethylamino-ethyl)-methyl-amino]-phenyl)-5-methyl-2-[(9H-purin-6-ylamino)-methyl]-3H-quinazolin-4-one; 5-chloro-3-(2-fluoro-phenyl)-2-[(9H-purin-6-ylamino)-methyl]-3H-quinazolin-4-one; 5-chloro-2-[(9H-purin-6-ylamino)-methyl]-3-o-tolyl-3H-quinazolin-4-one; 5-chloro-3-(2-chloro-phenyl)-2-[(9H-purin-6-ylamino)-methyl]-3H-quinazolin-4-one; 6-fluoro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; and the pharmaceutically acceptable salts and solvates thereof.

Formulations

The compounds according to the invention may be administered by oral, transdermal, inhalative, parenteral or sublingual route. The compounds according to the invention are present as active ingredients in conventional preparations, for example in compositions consisting essentially of an inert pharmaceutical carrier and an effective dose of the active substance, such as for example tablets, coated tablets, capsules, lozenges, powders, solutions, suspensions, emulsions, syrups, suppositories, transdermal systems etc. An effective dose of the compounds according to the invention is between 0.1 and 5000, preferably between 1 and 500, more preferably between 5-300 mg/dose for oral administration, and between 0.001 and 50, preferably between 0.1 and 10 mg/dose for intravenous. subcutaneous or intramuscular administration. Examples of inhalable formulations include inhalable powders, propellant-containing metered-dose aerosols or propellant-free inhalable solutions. Within the scope of the present invention the term propellant-free inhalable solutions also includes concentrates or sterile ready-to-use inhalable solutions. For use by inhalation it is preferable to use powders, ethanolic or aqueous solutions. For inhalation, according to the invention, solutions containing 0.01 to 1.0, preferably 0.1 to 0.5% active substance are suitable. It is also possible to use the compounds according to the invention as a solution for infusion, preferably in a physiological saline or nutrient saline solution.

The compounds according to the invention may be used on their own or in conjunction with other active substances according to the invention, optionally also in conjunction with other pharmacologically active substances. Suitable formulations include, for example, tablets, capsules, suppositories, solutions, syrups, emulsions or dispersible powders. Corresponding tablets may be obtained for example by mixing the active substance(s) with known excipients, for example inert diluents, such as calcium carbonate, calcium phosphate or lactose, disintegrants such as maize starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

The inhalable powders which may be used according to the invention may contain the active substance according to the invention either on its own or in admixture with suitable physiologically acceptable excipients.

If the active substances according to the invention are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. In some cases it may seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, micronised active substances according to the invention, preferably with an average particle size of 0.5 to 10 μm, more preferably from 1 to 5 μm, are added to the excipient mixture. Processes for producing the inhalable powders according to the invention by grinding and micronising and finally mixing the ingredients together are known from the prior art.

The inhalable powders according to the invention may be administered using inhalers known from the prior art.

Inhalation aerosols containing propellant gas according to the invention may contain active substances according to the invention dissolved in the propellant gas or in dispersed form. The propellant gases which may be used to prepare the inhalation aerosols are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The above-mentioned propellant gases may be used on their own or in admixture. Particularly preferred propellant gases are halogenated alkane derivatives selected from TG134a and TG227 and mixtures thereof.

The propellant-driven inhalation aerosols may also contain other ingredients such as co-solvents, stabilisers, surfactants, antioxidants, lubricants and pH adjusters. All these ingredients are known in the art.

The propellant-driven inhalation aerosols according to the invention mentioned above may be administered using inhalers known in the art (MDIs=metered dose inhalers).

Moreover, the active substances according to the invention may be administered in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is preferably up to 70 percent by volume, more particularly up to 60 percent by volume and most preferably up to 30 percent by volume. The remainder of the volume is made up of water. The solutions or suspensions containing the active substance according to the invention are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

The addition of editic acid (EDTA) or one of the known salts thereof, sodium edetate, as stabiliser or complexing agent may optionally be omitted in these formulations. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body. Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the active substance according to the invention, only benzalkonium chloride and sodium edetate. In another preferred embodiment, no sodium edetate is present.

A therapeutically effective daily dose is between 1 and 2000 mg, preferably 10-500 mg per adult.

The Examples which follow illustrate the present invention without restricting its scope:

Examples of Pharmaceutical Formulations

| A) Tablets | per tablet |
|---|---|
| active substance | 100 mg |
| lactose | 140 mg |
| maize starch | 240 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, granulated while wet and dried. The granulate, the rest of the corn starch and the magnesium stearate are screened and mixed together. The mixture is compressed to form tablets of a suitable shape and size.

| B) Tablets | per tablet |
|---|---|
| active substance | 80 mg |
| corn starch | 190 mg |
| lactose | 55 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| C) Coated tablets | per coated tablet |
|---|---|
| Active substance | 5 mg |
| Corn starch | 41.5 mg |
| Lactose | 30 mg |
| Polyvinylpyrrolidone | 3 mg |
| Magnesium stearate | 0.5 mg |
| | 80 mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in a known manner with a covering consisting essentially of sugar and talc. The finished coated tablets are polished with wax

| D) Capsules | per capsule |
|---|---|
| Active substance | 50 mg |
| Corn starch | 268.5 mg |
| Magnesium stearate | 1.5 mg |
| | 320 mg |

The substance and corn starch are mixed and moistened with water. The moist mass is screened and dried. The dry granules are screened and mixed with magnesium stearate. The finished mixture is packed into size 1 hard gelatine capsules.

| E) Ampoule solution | |
|---|---|
| active substance | 50 mg |
| sodium chloride | 50 mg |
| water for inj. | 5 ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

| F) Suppositories | |
|---|---|
| Active substance | 50 mg |
| Solid fat | 1650 mg |
| | 1700 mg |

The hard fat is melted. At 40° C. the ground active substance is homogeneously dispersed. It is cooled to 38° C. and poured into slightly chilled suppository moulds.

| G) Oral suspension | |
|---|---|
| active substance | 50 mg |
| hydroxyethylcellulose | 50 mg |
| sorbic acid | 5 mg |
| sorbitol (70%) | 600 mg |
| glycerol | 200 mg |
| flavouring | 15 mg |
| water ad | 5 ml |

Distilled water is heated to 70° C. Hydroxyethyl-cellulose is dissolved therein with stirring. After the addition of sorbitol solution and glycerol the mixture is cooled to ambient temperature. At ambient temperature, sorbic acid, flavouring and substance are added. To eliminate air from the suspension it is evacuated with stirring. and 50 mg of active substance.

| H) Metered-dose aerosol (suspension) | |
|---|---|
| active substance | 0.3 wt. % |
| sorbitolan trioleate | 0.6 wt. % |
| HFA134A:HFA227 2:1 | 99.1 wt. % |

The suspension is transferred into a conventional aerosol container with a metering valve. Preferably, 50 μl of suspension are delivered per spray. The active substance may also be metered in higher doses if desired.

| I) Metered-dose aerosol (solution) | | |
|---|---|---|
| active substance | 0.3 | wt. %. % |
| abs. ethanol | 20 | wt. % |
| aqueous HCl 0.01 mol/l | 2.0 | wt. % |
| HEA134A | 77.7 | wt. % |

The solution is produced in the usual way by mixing the individual ingredients together.

| J) Inhalable powder | |
|---|---|
| active substance | 80 μg |
| lactose monohydrate | ad 10 mg |

The powder for inhalation is produced in the usual way by mixing the individual ingredients together.

1. A compound of the formula (I), 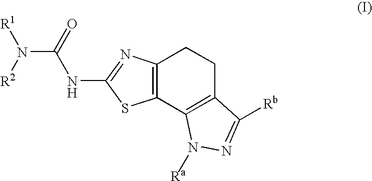
wherein
R^{a} denotes hydrogen or an optionally substituted group selected from among C₁-C₈-alkyl, C₂-C₈-alkenyl, C₂-C₈-alkynyl, C₃-C₈-cycloalkyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl and C₃-C₈-heterocycloalkyl-C₁-C₄-alkyl,
R^{b} denotes hydrogen or an optionally substituted group selected from among C₁-C₈-alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl, CONH₂, C₆-C₁₄-aryl-NH— and C₃-C₈-heterocycloalkyl-NH—;
R¹ denotes hydrogen or an optionally substituted group selected from among C₁-C₈-alkyl, C₃-C₈-cycloalkyl, C₂-C₈-alkenyl, C₂-C₈-alkynyl and C₆-C₁₄-aryl-C₁-C₅-alkyl-;
R² denotes hydrogen or an optionally substituted group selected from among C₁-C₈ alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₆-alkyl, C₉-C₁₃-spiro, C₃-C₈-heterocycloalkyl, C₃-C₈-heterocycloalkyl-C₁-C₆-alkyl- and C₆-C₁₄-aryl-C₁-C₆-alkyl-; or
R¹ and R² together form an optionally substituted five-, six- or seven-membered ring consisting of carbon atoms and optionally 1 to 2 heteroatoms, selected from among oxygen, sulphur and nitrogen, or
R¹ and R² together form an optionally substituted nine- to thirteen-membered spirocyclic ring, or
R² denotes a group selected from among general formulae (A1) to (A18) 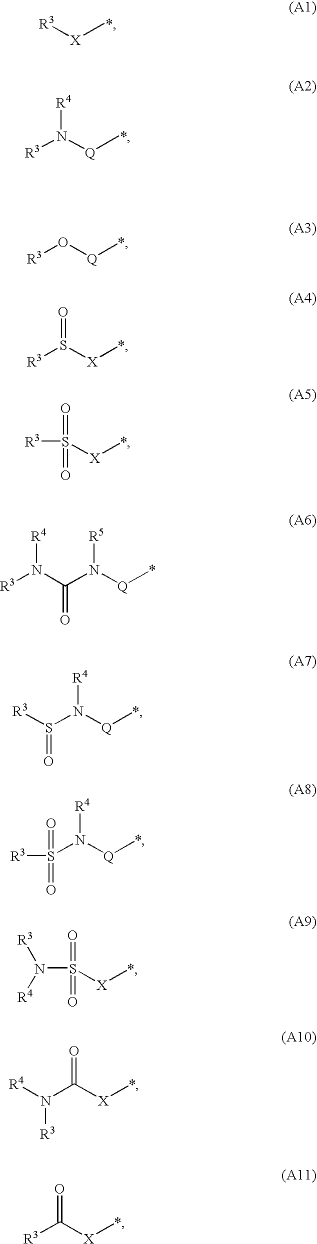 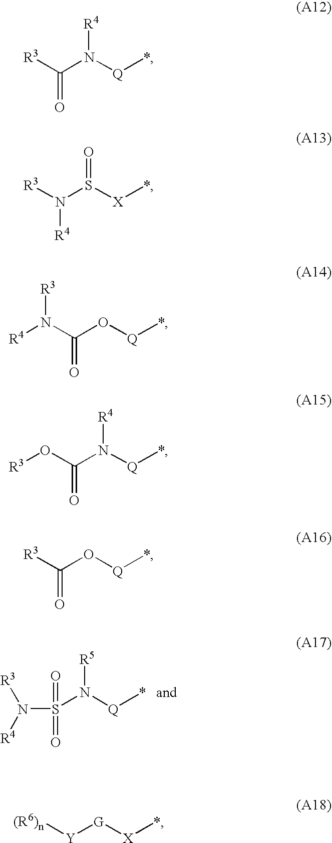
wherein
X and Y may be linked to the same or different atoms of G, and
X denotes a bond or an optionally substituted group selected from among C₁-C₇-alkylene, C₃-C₇-alkenylene and C₃-C₇-alkynylene, or
X together with R¹, R³ or R⁴ forms a C₁-C₇-alkylene bridge;
Y denotes a bond or optionally substituted C₁-C₄-alkylene;
Q denotes an optionally substituted group selected from among C₁-C₇-alkylene, C₃-C₇-alkenylene and C₃-C₇-alkynylene; or
Q together with R¹, R³ or R⁴ forms a C₁-C₇-alkylene bridge;
R³, R⁴, R⁵ which may be identical or different, denote hydrogen or an optionally substituted group selected from among C₁-C₈-alkyl, C₃-C₈-cycloalkyl, C₂-C₆-haloalkyl, C₁-C₄-alkyl-C₃-C₈-cycloalkyl, C₃-C₈-cycloalkyl-C₁-C₄-alkyl, NR⁷R⁸, NR⁷R⁸—C₁-C₄-alkyl, C₁-C₄-alkoxy, C₁-C₄-alkoxy-C₁-C₄-alkyl, C₆-C₁₄-aryl and C₅-C₁₀-heteroaryl; or in each case two of the substituents
R³, R⁴, R⁵ together form an optionally substituted five-, six- or seven-membered ring, consisting of carbon atoms and optionally 1-2 heteroatoms, selected from among oxygen, sulphur and nitrogen;
G denotes a saturated, partially saturated or unsaturated ring system consisting of 3-10 C atoms, wherein optionally up to 6 C atoms are replaced by heteroatoms selected from among nitrogen, oxygen and sulphur;
R⁶ which may be identical or different, denote hydrogen or an optionally substituted group selected from among ═O, C₁-C₈-alkyl, C₃-C₈-cycloalkyl, C₂-C₆-haloalkyl, C₆-C₁₄-aryl, C₅-C₁₀-heteroaryl, C₃-C₈-heterocycloalkyl, or
a group selected from among NR⁷R⁸, OR⁷, —CO—C₁-C₃-alkyl-NR⁷R⁸—O—C₁-C₃-alkyl-NR⁷R⁸, CONR⁷R⁸, NR⁷COR⁸, —CO—C₁-C₃-alkyl-NR⁷(CO)OR⁸, —O(CO)NR⁷R⁸, NR⁷(CO)NR⁸R⁹, NR⁷(CO)OR⁸, (CO)OR⁷, —O(CO)R⁷, COR⁷, (SO)R⁷, (SO₂)R⁷, (SO₂)NR⁷R⁸, NR⁷(SO₂)R⁸, NR⁷(SO₂)NR⁸R⁹, CN and halogen;
n denotes 1, 2 or 3
R⁷, R⁸, R⁹ which may be identical or different, denote hydrogen or an optionally substituted group selected from among C₁-C₈-alkyl, C₃-C₈-cycloalkyl, C₁-C₆-haloalkyl, C₁-C₄-alkyl-C₃-C₈-cycloalkyl, C₃-C₈-cycloalkyl-C₁-C₃-alkyl, C₆-C₁₄-aryl, C₁-C₄-alkyl-C₆-C₁₄-aryl, C₆-C₁₄-aryl-C₁-C₄-alkyl, C₃-C₈-heterocycloalkyl, C₁-C₅-alkyl-C₃-C₈-heterocycloalkyl, C₃-C₈-heterocycloalkyl-C₁-C₄-alkyl, C₁-C₄-alkyl(CO)— and C₁-C₄-alkyl-O(CO)—; or in each case two of the substituents
R⁷, R⁸, R⁹ together form an optionally substituted five-, six- or seven-membered ring, consisting of carbon atoms and optionally 1-2 heteroatoms, selected from among oxygen, sulphur and nitrogen;
optionally in the form of the tautomers, the racemates, the enantiomers, the diastereomers and the mixtures thereof, as well as optionally the pharmacologically acceptable acid addition salts, solvates and hydrates thereof,
with the proviso that the following compounds are excluded:
a) (1-phenyl-4,5-dihydro-1H-pyrazolo[3′,4′:3,4]benzo[1,2-d]thiazol-7-yl)-hydrazinecarboxamide
b) 1-(2-dimethylamino-ethyl)-3-(1-phenyl-4,5-dihydro-1H-pyrazolo[3′,4′:3,4]benzo[1,2-d]thiazol-7-yl)-urea
c) 1-(2-morpholin-4-yl-ethyl)-3-(1-phenyl-4,5-dihydro-1H-pyrazolo[3′,4′:3,4]benzo[1,2-d]thiazol-7-yl)-urea
d) 1-ethyl-3-(1-phenyl-4,5-dihydro-1H-pyrazolo[3′,4′:3,4]benzo[1,2-d]thiazol-7-yl)-urea
e) 1-methyl-3-(1-phenyl-4,5-dihydro-1H-pyrazolo[3′,4′:3,4]benzo[1,2-d]thiazol-7-yl)-urea
f) 1,1-dimethyl-3-(1-phenyl-4,5-dihydro-1H-pyrazolo[3′,4′:3,4]benzo[1,2-d]thiazol-7-yl)-urea
g) morpholine-4-carboxylic acid (1-phenyl-4,5-dihydro-1H-pyrazolo[3′,4′:3,4]benzo[1,2-d]thiazol-7-yl)-amide
h) [1-(2-chloro-phenyl)-3-isopropyl-4,5-dihydro-1H-pyrazolo[3′,4′:3,4]benzo[1,2-d]thiazol-7-yl]-urea
i) N-(5.8-dihydro-4H-[1,3]thiazolo[4,5-g]indol-2-yl)-N′-ethylurea
j) N-ethyl-N′-(8-methyl-5.8-dihydro-4H-[1,3]thiazolo[4,5-g]indol-2-yl)urea
k) tert-butyl {4-[3-(1-phenyl-4,5-dihydro-1H-pyrazolo[3′,4′:3,4]benzo[1,2-d]thiazol-7-yl)-ureido]-but-2-ynyl}-carbamate
l) 1-(4-amino-but-2-ynyl)-3-(1-phenyl-4,5-dihydro-1H-pyrazolo[3′,4′:3,4]benzo[1,2-d]thiazol-7-yl)-urea or
m) (1-phenyl-4,5-dihydro-1H-pyrazolo[3′,4′:3,4]benzo[1,2-d]thiazol-7-yl)-urea.

2. The compound according to claim 1, wherein
X, Y, Q and G may have the meaning specified and
R^{a} denotes hydrogen or a group selected from among C₁-C₈-alkyl, C₂-C₈-alkenyl, C₂-C₈-alkynyl, C₃-C₈-cycloalkyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl and C₃-C₈-heterocycloalkyl-C₁-C₄-alkyl, which may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, C₁-C₄-alkoxy, CN, NO₂, NR¹⁰R¹¹, OR¹⁰, COR¹⁰, COOR¹⁰, CONR¹⁰R¹¹, NR¹⁰COR¹¹, NR¹⁰(CO)NR¹¹R¹², O(CO)NR¹⁰R¹¹, NR¹⁰(CO)OR¹¹, SO₂R¹⁰, SOR¹⁰, SO₂NR¹⁰R¹¹, NR¹⁰SO₂NR¹¹R¹² NR¹⁰SO₂R¹¹;
R¹⁰, R¹¹, R¹² which may be identical or different, denote hydrogen or a group selected from among C₁-C₆-alkyl, C₂-C₆-alkenyl, C₂-C₆-alkynyl, C₃-C₈-cycloalkyl and C₁-C₆ haloalkyl; or
in each case two of the groups
R¹⁰, R¹¹, R¹² together form a five-, six- or seven-membered ring, consisting of carbon atoms and optionally 1-2 heteroatoms, selected from among oxygen, sulphur and nitrogen;
R^{b} denotes hydrogen or a group selected from among C₁-C₈-alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl, CONH₂, C₆-C₁₄-aryl-NH, C₃-C₈-heterocycloalkyl-NH, which may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, OMe, CN, NH₂, NHMe, NMe₂;
R¹ denotes hydrogen or a group selected from among C₁-C₈-alkyl, C₃-C₈-cycloalkyl, C₂-C₈-alkenyl, C₂-C₈-alkynyl and C₆-C₁₄-aryl-C₁-C₅-alkyl, which may optionally be substituted by one or more of the groups, which may be identical or different, selected from among halogen, NH₂, OH, CN, C₁-C₆-alkyl, OMe, —NH(CO)-alkyl and —(CO)O-alkyl,
R² denotes hydrogen or a group selected from among C₁-C₈ alkyl, C₃-C₈-cycloalkyl, C₂-C₈-alkenyl, C₃-C₈-cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₆-alkyl, C₉-C₁₃-spiro, C₃-C₈-heterocycloalkyl, C₃-C₈-heterocycloalkyl-C₁-C₆-alkyl- and C₆-C₁₄-aryl-C₁-C₆-alkyl, which may optionally be substituted by one or more of the groups, which may be identical or different, selected from among halogen, NH₂, OH, CN, C₁-C₆-alkyl, OMe, —NH(CO)-alkyl and —(CO)O-alkyl, or
R¹ and R² together form a five-, six- or seven-membered ring consisting of carbon atoms and optionally 1 to 2 heteroatoms, selected from among oxygen, sulphur and nitrogen, which may optionally be substituted by one or more of the groups, which may be identical or different, selected from among halogen, NH₂, OH, CN, C₁-C₆-alkyl, OMe, —NH(CO)-alkyl and —(CO)O-alkyl, or
R¹ and R² together form an optionally substituted nine- to thirteen-membered spirocyclic ring, or
R² denotes a group selected from among general formulae (A1) to (A18) 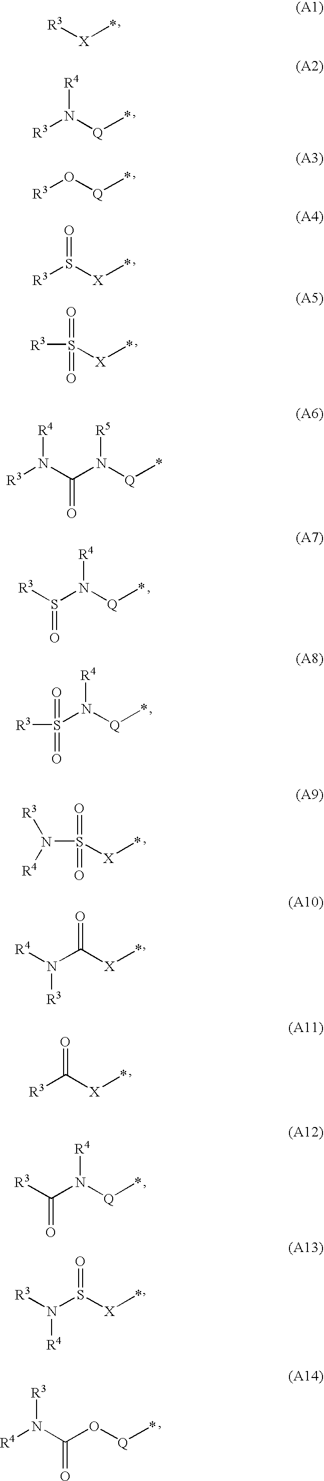 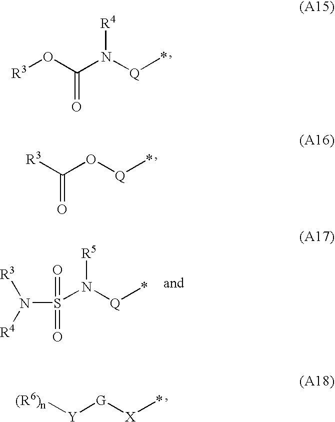
wherein
R³, R⁴, R⁵ which may be identical or different, denote hydrogen or a group selected from among C₁-C₈-alkyl, C₃-C₈-cycloalkyl, C₂-C₆-haloalkyl, C₁-C₄-alkyl-C₃-C₈-cycloalkyl, C₃-C₈-cycloalkyl-C₁-C₄-alkyl, NR⁷R⁸, NR⁷R⁸—C₁-C₄-alkyl, C₁-C₄-alkoxy, C₁-C₄-alkoxy-C₁-C₄-alkyl, C₆-C₁₄-aryl and C₅-C₁₀-heteroaryl, which may optionally be substituted by one or more of the groups, which may be identical or different, selected from among halogen, NH₂, OH, CN, NR⁹R¹⁰, —NH(CO)—C₁-C₄-alkyl and MeO, or in each case two of the substituents
R³, R⁴, R⁵ together form a five-, six- or seven-membered ring, consisting of carbon atoms and optionally 1-2 heteroatoms, selected from among oxygen, sulphur and nitrogen; which may optionally be substituted by one or more of the groups, which may be identical or different, selected from among halogen, NH₂, OH, CN, NR⁹R¹⁰, —NH(CO)—C₁-C₄-alkyl and MeO,
R⁶ which may be identical or different, denote hydrogen or a group, selected from among, C₁-C₈-alkyl, C₃-C₈-cycloalkyl, C₂-C₆-haloalkyl, C₆-C₁₄-aryl, C₅-C₁₀-heteroaryl and C₃-C₈-heterocycloalkyl, which may optionally be substituted by one or more of the groups, which may be identical or different, selected from among, NH₂, NHMe, NMe₂, OH, OMe, CN and C₁-C₆-alkyl and —(CO)O—C₁-C₆-alkyl, or
a group selected from among ═O, NR⁷R⁸, OR⁷, —CO—C₁-C₃-alkyl-NR⁷R⁸, —O—C₁-C₃-alkyl-NR⁷R⁸, CONR⁷R⁸, NR⁷COR⁸, —CO—C₁-C₃-alkyl-NR⁷(CO)OR⁸, —O(CO)NR⁷R⁸, N R⁷(CO)NR⁸R⁹, NR⁷(CO)OR⁸, (CO)OR⁷, —O(CO)R⁷, COR⁷, (SO)R⁷, (SO₂)R⁷, (SO₂)NR⁷R⁸, NR⁷(SO₂)R⁸, NR⁷(SO₂)NR⁸R⁹, CN and halogen;
n denotes 1, 2 or 3
R⁷, R⁸, R⁹ which may be identical or different, denote hydrogen or a group selected from among C₁-C₈-alkyl, C₃-C₈-cycloalkyl, C₂-C₆-haloalkyl, C₁-C₄-alkyl-C₃-C₈-cycloalkyl, C₃-C₈-cycloalkyl-C₁-C₃-alkyl, C₆-C₁₄-aryl, C₁-C₄-alkyl-C₆-C₁₄-aryl, C₆-C₁₄-aryl-C₁-C₄-alkyl, C₃-C₈-heterocycloalkyl, C₁-C₅-alkyl-C₃-C₈-heterocycloalkyl, C₃-C₈-heterocycloalkyl-C₁-C₄-alkyl, C₁-C₄-alkyl(CO)— and C₁-C₄-alkyl-O(CO), which may optionally be substituted by one or more of the groups, which may be identical or different, selected from among halogen, NH₂, OH, CN, OMe, NHMe, NMe₂, C₁-C₆-alkyl and (CO)O C₁-C₆-alkyl, or in each case two of the substituents
R⁷, R⁸, R⁹ together form a five-, six- or seven-membered ring, consisting of carbon atoms and optionally 1-2 heteroatoms, selected from among oxygen, sulphur and nitrogen; which may optionally be substituted by one or more of the groups, which may be identical or different, selected from among halogen, NH₂, OH, CN, OMe, NHMe, NMe₂, C₁-C₆-alkyl and (CO)OC₁-C₆-alkyl.

3. The compounds according to claim 2, wherein
R^{a} and R¹ to R¹² may have the meaning specified and
R^{b} denotes a group selected from among C₁-C₈-alkyl, C₃-C₈-cycloalkyl, C₂-C₈-alkenyl, C₃-C₈-Cycloalkenyl, C₁-C₆-haloalkyl, C₆-C₁₄-aryl, C₆-C₁₄-aryl-C₁-C₅-alkyl, C₅-C₁₀-heteroaryl, C₃-C₈-cycloalkyl-C₁-C₄-alkyl, C₃-C₈-cycloalkenyl-C₁-C₄-alkyl, C₅-C₁₀-heteroaryl-C₁-C₄-alkyl, spiro, C₃-C₈-heterocycloalkyl, CONH₂, C₆-C₁₄-aryl-NH— and C₃-C₈-heterocycloalkyl-NH, which may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, OMe, CN, NH₂, NHMe and NMe₂.

4. The compound according to claim 3, wherein
R¹ to R¹² may have the meaning specified and
R^{a} denotes C₆-C₁₄-aryl or a saturated ring system consisting of 5-6 C atoms, wherein optionally up to 4 C atoms are replaced by nitrogen atoms, wherein R^{a} may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, C₁-C₄-alkoxy, CN, NO₂, NR¹⁰R¹¹, OR¹⁰, COR¹⁰, COOR¹⁰, CONR¹⁰R¹¹, NR¹⁰COR¹¹, NR¹⁰(CO)NR¹¹R¹²O(CO)NR¹⁰R¹¹, NR¹⁰(CO)OR¹¹, SO₂R¹⁰, SOR¹⁰, SO₂NR¹⁰R¹¹, NR¹⁰SO₂NR¹¹R¹² and NR¹⁰SO₂R¹¹;
R^{b} denotes hydrogen or a group selected from among C₃-C₈-cycloalkyl, C₆-C₁₄-aryl, C₅-C₁₀-heteroaryl, C₆-C₁₄-aryl-NH, which may optionally be substituted by one or more of the groups, which may be identical or different, selected from among C₁-C₆-alkyl, C₂-C₆-alkenyl, C₂-C₆-alkynyl, C₃-C₈-cycloalkyl, C₁-C₆-haloalkyl, halogen, OH, OMe, CN, NH₂, NHMe and NMe₂.

5. The compound according to claim 4, wherein
R^{a} and R^{b} may have the meaning specified and
R¹ denotes hydrogen, C₁-C₅-alkyl or C₃-C₈-cycloalkyl,
R² denotes hydrogen, C₁-C₅-alkyl or C₃-C₈-cycloalkyl, or
R¹ and R² together form an optionally substituted five- or six-membered ring consisting of carbon atoms and optionally 1 to 2 nitrogen atoms, or
R¹ and R² together form an optionally substituted nine- to thirteen-membered spirocyclic ring, or
R¹, R² which may be identical or different, denote a group selected from among general formulae (A2), (A3), (A8), (A10), (A11) and (A12), wherein
X denotes a bond or an optionally substituted C₁-C₃-alkylene, or
X together with R¹, R³ or R⁴ forms a 5- or 6-membered heterocyclic group with R¹, R³ or R⁴;
Q denotes an optionally substituted C₁-C₃-alkylene, or
Q together with R¹, R³ or R⁴ forms a C₁-C₇-alkylene bridge;
R³, R⁴, R⁵ which may be identical or different, denote hydrogen or an optionally substituted group selected from among C₁-C₄-alkyl, C₁-C₄-alkoxy, C₃-C₆-cycloalkyl and C₅-C₁₀-heteroaryl, or in each case two of the substituents
R³, R⁴, R⁵ together form an optionally substituted five- or six-membered ring, consisting of carbon atoms and optionally 1-2 heteroatoms, selected from among oxygen and nitrogen.

6. The compound according to claim 4, wherein
R^{a} and R^{b} may have the meaning specified and
R¹ denotes H, or Me,
R² denotes hydrogen or a group of general formulae (A18), wherein
X denotes a bond or an optionally substituted group selected from among C₁-C₇-alkylene, C₃-C₇-alkenylene and C₃-C₇-alkynylene, or
X together with R¹ forms a C₁-C₇-alkylene bridge
Y denotes a bond or methylene, ethylene;
X and Y may be linked to the same or different atoms of G, and
G denotes a saturated, partially saturated or unsaturated ring system consisting of 3-10 C atoms, wherein optionally up to 6 C atoms are replaced by heteroatoms selected from among nitrogen, oxygen and sulphur;
R⁶ which may be identical or different, denote hydrogen or an optionally substituted group selected from among ═O, C₁-C₄-alkyl, C₃-C₆-cycloalkyl, C₆-C₁₄-aryl, C₅-C₆-heterocycloalkyl, and C₅-C₆-heteroaryl or a group selected from among NR⁷R⁸, OR⁷, —O—C₁-C₃-alkyl-NR⁷R⁸, CONR⁷R⁸, CO—C₁-C₃-alkyl-NR⁷R⁸NR⁷COR⁸, NR⁷(CO)OR⁸, —CO—C₁-C₃-alkyl-NR⁷(CO)OR⁸, NR⁷(CO)NR⁸R⁹, NR⁷(CO)OR⁸, (CO)OR⁷, COR⁷, (SO₂)R⁷ and CN,
n denotes 1 or 2
R⁷, R⁸, R⁹ which may be identical or different, denote hydrogen or an optionally substituted group selected from among C₁-C₅-alkyl, C₁-C₄-alkyl-C₆-C₁₄-aryl, C₃-C₆-heterocycloalkyl, C₁-C₅-alkyl-C₃-C₈-heterocycloalkyl, or in each case two of the substituents
R⁷, R⁸, R⁹ together form an optionally substituted five- or six-membered ring, consisting of carbon atoms and optionally 1-2 heteroatoms, selected from among oxygen and nitrogen.

7. A method of treating a disease or condition chosen from chronic bronchitis, acute bronchitis, bronchitis caused by bacterial or viral infection or fungi or helminths, allergic bronchitis, toxic bronchitis, chronic obstructive bronchitis (COPD), asthma (intrinsic or allergic), paediatric asthma, bronchiectasis, allergic alveolitis, allergic or non-allergic rhinitis, chronic sinusitis, cystic fibrosis or mucoviscidosis, alpha-1-antitrypsin deficiency, cough, pulmonary emphysema, interstitial lung diseases, alveolitis, hyperreactive airways, nasal polyps, pulmonary oedema, pneumonitis of different origins, e.g. radiation-induced or caused by aspiration, or infectious pneumonitis, collagenoses such as lupus erythematodes, systemic sclerodermy, sarcoidosis and Boeck's disease comprising administering a therapeutically effective amount of a compound according to claim 1.

8. A method of treating a disease or condition chosen from psoriasis, contact dermatitis, atopic dermatitis, alopecia areata (circular hair loss), erythema exsudativum multiforme (Stevens-Johnson Syndrome), dermatitis herpetiformis, sclerodermy, vitiligo, nettle rash (urticaria), lupus erythematodes, follicular and surface pyodermy, endogenous and exogenous acne, acne rosacea and other inflammatory and allergic or proliferative skin diseases comprising administering a therapeutically effective amount of a compound according to claim 1.

9. A method of treating a disease or condition chosen from inflammation of the conjunctiva (conjunctivitis) of various kinds, such as e.g. caused by infection with fungi or bacteria, allergic conjunctivitis, irritable conjunctivitis, drug-induced conjunctivitis, keratitis and uveitis comprising administering a therapeutically effective amount of a compound according to claim 1.

10. A method of treating a disease or condition chosen from among allergic rhinitis, allergic sinusitis and nasal polyps comprising administering a therapeutically effective amount of a compound according to claim 1.

11. A method of treating a disease or condition chosen from Crohn's disease, ulcerative colitis, systemic lupus erythematodes, chronic hepatitis, multiple sclerosis, rheumatoid arthritis, psoriatic arthritis, osteoarthritis, rheumatoid spondylitis comprising administering a therapeutically effective amount of a compound according to claim 1.

12. A method of treating diseases or conditions chosen from glomerulonephritis, interstitial nephritis and idiopathic nephrotic syndrome comprising administering a therapeutically effective amount of a compound according to claim 1.

13. A pharmaceutical composition comprising a pharmaceutically effective amount of a compound according to claim 1.

14. The pharmaceutical composition according to claim 13 for inhalative administration.

15. The pharmaceutical composition according to claim 13 for oral administration.

16. The pharmaceutical composition according to claim 13, comprising as a further active substance, one or more compounds which are selected from the categories of the betamimetics, anticholinergics, corticosteroids, other PDE4-inhibitors, LTD4-antagonists, EGFR-inhibitors, dopamine agonists, H1-antihistamines, PAF-antagonists and PI3-kinase inhibitors or double or triple combinations thereof.
